SEQUENCE LISTING (75 sequences)

SEQ ID NO: 1 - protein, 5 aa, synthetic construct
SYAMS

SEQ ID NO: 2 - protein, 11 aa, synthetic construct
GLRVGVLGFDY

SEQ ID NO: 3 - protein, 17 aa, synthetic construct
AINAGGGSTSYADSVKG

SEQ ID NO: 4 - protein, 120 aa, synthetic construct
EVQLLESGGGLVQPGGSLRLSCAASGFTFSSYAMSWVRQAPGKGLEWVSAINAGGGSTSYADSVKGRFTISRDNSKNTLYLQMNSLRAEDTAVYYCAKGLRVGVLGFDYWGQGTLVTVSS

SEQ ID NO: 5 - protein, 17 aa, synthetic construct
AINNGGGSTSYADSVKG

SEQ ID NO: 6 - protein, 120 aa, synthetic construct
EVQLLESGGGLVQPGGSLRLSCAASGFTFSSYAMSWVRQAPGKGLEWVSAINNGGGSTSYADSVKGRFTISRDNSKNTLYLQMNSLRAEDTAVYYCAKGLRVGVLGFDYWGQGTLVTVSS

SEQ ID NO: 7 - protein, 11 aa, synthetic construct
DGNNIGSKSAQ

SEQ ID NO: 8 - protein, 9 aa, synthetic construct
QVWDGSAAV

SEQ ID NO: 9 - protein, 7 aa, synthetic construct
ADEYRPE

SEQ ID NO: 10 - protein, 106 aa, synthetic construct
SYELTQPLSVSVALGQTARITCDGNNIGSKSAQWYQQKPGQAPVLVIYADEYRPEGIPERFSGSNSGNTATLTISRAQAGDEADYYCQVWDGSAAVFGGGTQLTVL

SEQ ID NO: 11 - protein, 5 aa, synthetic construct
SYDMS

SEQ ID NO: 12 - protein, 11 aa, synthetic construct
THGIGTLGFGS

SEQ ID NO: 13 - protein, 17 aa, synthetic construct
AIAQFQHWTYYADSVKG

SEQ ID NO: 14 - protein, 120 aa, synthetic construct
EVQLLESGGGLVQPGGSLRLSCAASGFTFRSYDMSWVRQAPGKGLEWVSAIAQFQHWTYYADSVKGRFTISRDNSKNTLYLQMNSLRAEDTAVYYCATTHGIGTLGFGSWGQGTLVTVSS

SEQ ID NO: 15 - protein, 5 aa, synthetic construct
SFAMS

SEQ ID NO: 16 - protein, 12 aa, synthetic construct
GGATVWGRFHDY

SEQ ID NO: 17 - protein, 12 aa, synthetic construct
GINSGDGSSVKG

SEQ ID NO: 18 - protein, 116 aa, synthetic construct
EVQLLESGGGLVQPGGSLRLSCAASGFTFSSFAMSWVRQAPGKGLEWVSGINSGDGSSVKGRFTISRDNSKNTLYLQMNSLRAEDTAVYYCAKGGATVWGRFHDYWGQGTLVTVSS

SEQ ID NO: 19 - protein, 14 aa, synthetic construct
TGSSSNIGSGSYLS

SEQ ID NO: 20 - protein, 11 aa, synthetic construct
GCYDSKLSTYV

SEQ ID NO: 21 - protein, 7 aa, synthetic construct
NANNRAS

SEQ ID NO: 22 - protein, 111 aa, synthetic construct
QSVLTQPPSVSGAPGQRVTISCTGSSSNIGSGSYLSWYQQLPGTAPKLLIYNANNRASGVPDRFSGSKSGTSASLAITGLQAEDEADYYCGCYDSKLSTYVFGGGTKLTVL

SEQ ID NO: 23 - protein, 16 aa, synthetic construct
AIKWHRITYYADSVKG

SEQ ID NO: 24 - protein, 119 aa, synthetic construct
EVQLLESGGGLVQPGGSLRLSCAASGFTFRSYDMSWVRQAPGKGLEWVSAIKWHRITYYADSVKGRFTISRDNSKNTLYLQMNSLRAEDTAVYYCATTHGIGTLGFGSWGQGTLVTVSS

SEQ ID NO: 25 - protein, 142 aa, synthetic construct
MSLLPVPYTEAASLSTGSTVTIKGRPLACFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNVSYLKR

SEQ ID NO: 26 - protein, 10 aa, synthetic construct
EPKSCDKTHT

SEQ ID NO: 27 - protein, 5 aa, synthetic construct
CPPCP

SEQ ID NO: 28 - protein, 8 aa, synthetic construct
APELLGGP

SEQ ID NO: 29 - protein, 12 aa, synthetic construct
ELKTPLGDTTHT

SEQ ID NO: 30 - protein, 50 aa, synthetic construct
CPRCPEPKSCDTPPPCPRCPEPKSCDTPPPCPRCPEPKSCDTPPPCPRCP

SEQ ID NO: 31 - protein, 8 aa, synthetic construct
APELLGGP

SEQ ID NO: 32 - protein, 7 aa, synthetic construct
ESKYGPP

SEQ ID NO: 33 - protein, 5 aa, synthetic construct
CPSCP

SEQ ID NO: 34 - protein, 8 aa, synthetic construct
APEFLGGP

SEQ ID NO: 35 - skipped

SEQ ID NO: 36 - protein, 10 aa, synthetic construct
CCVECPPPCP

SEQ ID NO: 37 - protein, 7 aa, synthetic construct
APPVAGP

SEQ ID NO: 38 - protein, 7 aa, synthetic construct
IKWHRIT

SEQ ID NO: 39 - protein, 7 aa, synthetic construct
IKHQVIT

SEQ ID NO: 40 - protein, 7 aa, synthetic construct
IKPVGIT

SEQ ID NO: 41 - protein, 6 aa, synthetic construct
ISGALT

SEQ ID NO: 42 - protein, 6 aa, synthetic construct
IHTPMT

SEQ ID NO: 43 - protein, 6 aa, synthetic construct
IKRPWT

SEQ ID NO: 44 - protein, 7 aa, synthetic construct
IKLGTFT

SEQ ID NO: 45 - protein, 8 aa, synthetic construct
IAQFQHWT

SEQ ID NO: 46 - protein, 8 aa, synthetic construct
IRTFEHHT

SEQ ID NO: 47 - protein, 8 aa, synthetic construct
ISKMPHLT

SEQ ID NO: 48 - protein, 8 aa, synthetic construct
IQKTIVPT

SEQ ID NO: 49 - protein, 8 aa, synthetic construct
IKNGGGIT

SEQ ID NO: 50 - protein, 17 aa, synthetic construct
GLEWVSGINSGDGSSVK

SEQ ID NO: 51 - protein, 21 aa, synthetic construct
GLEWVSAINSGGGTYYADTVK

SEQ ID NO: 52 - protein, 22 aa, synthetic construct
GLEWVSSIINGGESTTYRDSVK

SEQ ID NO: 53 - protein, 22 aa, synthetic construct
GLEWVSSISWNGQNTYYADTGK

SEQ ID NO: 54 - protein, 21 aa, synthetic construct
GLEWVSAIKWHRITYYADSVK

SEQ ID NO: 55 - protein, 22 aa, synthetic construct
GLEWVSAIAQFQHWTYYADSVK

SEQ ID NO: 56 - DNA, 35 bp, synthetic construct
  source 1..35: mol_type = other DNA
actggcggcccagccggccatggccgaagtgcagc

SEQ ID NO: 57 - DNA, 25 bp, synthetic construct
  source 1..25: mol_type = other DNA
gtcagtgctagcagagctaacggtg

SEQ ID NO: 58 - DNA, 49 bp, synthetic construct
  source 1..49: mol_type = other DNA
agagtcggcgtagtaggtgatmnnmnnmnncttgatggcggacacccat

SEQ ID NO: 59 - DNA, 21 bp, synthetic construct
  source 1..21: mol_type = other DNA
atcacctactacgccgactct

SEQ ID NO: 60 - DNA, 55 bp, synthetic construct
  source 1..55: mol_type = other DNA
acagagtcggcgtagtaggtmnnmnnmnnmnnmnngatggcggacacccattcaa

SEQ ID NO: 61 - DNA, 52 bp, synthetic construct
  source 1..52: mol_type = other DNA
acagagtcggcgtagtaggtmnnmnnmnnmnngatggcggacacccattcaa

SEQ ID NO: 62 - DNA, 20 bp, synthetic construct
  source 1..20: mol_type = other DNA
acctactacgccgactctgt

SEQ ID NO: 63 - DNA, 58 bp, synthetic construct
  source 1..58: mol_type = other DNA
acagagtcggcgtagtaggtmnnmnnmnnmnnmnnmnngatggcggacacccattcaa

SEQ ID NO: 64 - DNA, 35 bp, synthetic construct; identical to SEQ ID NO: 56
  source 1..35: mol_type = other DNA

SEQ ID NO: 65 - DNA, 25 bp, synthetic construct
  source 1..25: mol_type = other DNA
gtcagtgctagcagagctaacggtg

SEQ ID NO: 66 - protein, 19 aa, synthetic construct
EVQLLESGGGLVQPGGSLR

SEQ ID NO: 67 - protein, 11 aa, synthetic construct
NTLYLQMNSLR

SEQ ID NO: 68 - protein, 63 aa, synthetic construct
DYFPEPVTVSWNSGALTSGVHTFPAVLQSSGLYSLSSVVTVPSSSLGTQTYICNVNHKPSNTK

SEQ ID NO: 69 - protein, 11 aa, synthetic construct
ITCDGNNIGSK

SEQ ID NO: 70 - protein, 9 aa, synthetic construct
LLIYNANNR

SEQ ID NO: 71 - protein, 34 aa, synthetic construct
AQAGDEADYYCQVWDGSAAVFGGGTQLTVLGQPK

SEQ ID NO: 72 - protein, 7 aa, synthetic construct
ADSSPVK

SEQ ID NO: 73 - skipped

SEQ ID NO: 74 - protein, 6 aa, synthetic construct
KNGGGI

SEQ ID NO: 75 - protein, 7 aa, synthetic construct
IKXXXIT

(19) United States
(12) Patent Application Publication
(10) Pub. No.: US 2025/0059285 A1
(43) Pub. Date: Feb. 20, 2025

(54) GALECTIN-10 ANTIBODIES

(71) Applicant: argenx BV, Ghent (BE)

(72) Inventors: Paul Sebastian VAN DER WONING, Ghent (BE); Jean-Michel PERCIER, Ghent (BE)

(21) Appl. No.: 18/771,761

(22) Filed: Jul. 12, 2024

Related U.S. Application Data
(63) Continuation of application No. PCT/EP2023/051100, filed on Jan. 18, 2023.

(30) Foreign Application Priority Data
Jan. 18, 2022 (GB) 2200597.9

(51) Int. Cl. C07K 16/28

(52) U.S. Cl. CPC C07K 16/2851; C07K 2317/24; C07K 2317/33; C07K 2317/55; C07K 2317/622; C07K 2317/92; C07K 2317/94

(57) ABSTRACT

The present invention relates to antibodies and antigen binding fragments thereof that bind to the protein galectin-10, particularly human galectin-10. The galectin-10 antibodies and antigen binding fragments of the invention, disrupt the crystallization of galectin-10 and are therefore useful in methods of preventing and treating diseases and conditions wherein the pathology is linked to the formation/presence of Charcot-Leyden crystals (CLCs).

### CROSS-REFERENCE TO RELATED APPLICATIONS

This application is a Continuation of International Application No. PCT/EP2023/051100, filed Jan. 18, 2023, which claims priority to GB2200597.9, filed Jan. 18, 2022. The entire contents of these applications are incorporated herein by reference in their entirety.

### SEQUENCE LISTING

The content of the electronically submitted Sequence Listing (Name: 18771761_1_1.xml; Size: 65,988 bytes; and Date of Creation: Jan. 16, 2023) in this application is incorporated by reference in its entirety.

### FIELD OF THE INVENTION

The present invention relates to antibodies and antigen binding fragments thereof that bind to the protein galectin-10, particularly human galectin-10. The galectin-10 antibodies and antigen binding fragments of the invention, disrupt the crystallization of galectin-10 and are therefore useful in methods of preventing and treating diseases and conditions wherein the pathology is linked to the formation/presence of Charcot-Leyden crystals (CLCs).

### BACKGROUND TO THE INVENTION

Charcot-Leyden crystals (CLCs) were first described in 1853 and are microscopic, colourless crystals found in patients with certain conditions including allergic asthma and parasitic infections. CLCs are frequently observed in human tissues and secretions associated with an eosinophilic inflammatory response. In addition to asthma and parasitic infections, these crystals have been found in patients with cancer, for example myeloid leukemia. Structurally, CLCs accumulate as extracellular hexagonal bipyramidal crystals with a length of 20-40 μm and a width of 2-4 μm. The protein forming these crystals has been identified as galectin-10.

Galectin-10 (also known as Charcot Leyden Crystal Protein) is a small (16.5 kDa), auto-crystallizing, hydrophobic, glycan-binding protein expressed abundantly in the bone marrow, primarily by eosinophils (Chua et al. (2012) PLOS One. 7 (8): e42549). Galectin-10 is also produced to a lesser extent by basophils and Foxp3-positive Tregs (Kubach et al. (2007) Blood 110 (5): 1550-8). This protein is among the most abundant of eosinophil constituents, representing 7%-10% of total cellular protein. Galectin-10 is only found in humans and non-human primates, it lacks a secretion peptide signal and transmembrane domain, and is secreted under certain conditions by non-classical and novel apocrine mechanisms.

Despite abundant reports showing the appearance of CLCs in tissues from patients with eosinophilic disorders, the common view was that these crystals were merely a marker of eosinophil demise. This was view was ultimately challenged by studies demonstrating that CLCs boost type 2 immunity in a mouse model of house dust mite (HDM)-induced asthma (Persson et al., Science (2019)). Additionally, it has been observed that CLCs are abundant in the sticky mucus of patients with Aspergillosis and CRSwNP, which suggests that CLCs contribute to the viscoelasticity of mucus (Su J., Molecules (2018)).

### SUMMARY OF THE INVENTION

The recent findings implicating galectin-10 and CLC formation in diseases indicates that it is a target for therapeutics. It is reported herein that galectin-10 crystals can be dissolved by the administration of galectin-10 antibodies. Importantly, the galectin-10 antibodies reported herein retain activity and remain stable even after storage for 4 weeks at elevated temperatures such as 37° C. Taken together, this demonstrates that the galectin-10 antibodies reported herein can be used to treat conditions and disorders where the pathology is linked to the presence of CLCs.

In a first aspect, the invention provides an antibody or antigen binding fragment that binds to galectin-10, wherein the antibody or antigen binding fragment comprises a variable heavy chain (VH) domain and a variable light chain (VL) domain wherein:
(i) the VH domain comprises the CDR sequences of HCDR3 comprising or consisting of SEQ ID NO: 2; HCDR2 comprising or consisting of SEQ ID NO: 3; HCDR1 comprising or consisting of SEQ ID NO: 1; and
(ii) the VL domain comprises the CDR sequences of LCDR3 comprising or consisting of SEQ ID NO: 8; LCDR2 comprising or consisting of SEQ ID NO: 9; LCDR1 comprising or consisting of SEQ ID NO: 7.

In certain embodiments, the VH domain comprises the amino acid sequence of SEQ ID NO: 4 or an amino acid sequence at least 90%, 95%, 97%, 98% or 99% identical thereto; and the VL domain comprises the amino acid sequence of SEQ ID NO: 10 or an amino acid sequence at least 90%, 95%, 97%, 98% or 99% identical thereto.

In certain embodiments, the VH domain comprises the amino acid sequence of SEQ ID NO: 4; and the VL domain comprises the amino acid sequence of SEQ ID NO: 10.

In certain embodiments, the antigen binding fragment is selected from the group consisting of: a single chain antibody (scFv); a F(ab′)2 fragment; a Fab fragment; an Fd fragment; an Fv fragment; a one-armed (monovalent) antibody; diabodies, triabodies, tetrabodies, or any antigen binding molecule formed by combination, assembly or conjugation of such antigen binding fragments. In a preferred embodiment, the antigen binding fragment is a Fab fragment.

In a further aspect, the invention provides an isolated polynucleotide or polynucleotides which encode the antibody or antigen binding fragment as described herein, including polynucleotides encoding the VH and/or VL domains of the antibodies and antigen binding fragments described herein.

In another aspect, provided herein is an expression vector comprising the polynucleotide or polynucleotides as described herein operably linked to regulatory sequences which permit expression of the antibody, antigen binding fragment, variable heavy chain domain or variable light chain domain in a host cell or cell-free expression system.

In a further aspect, the invention provides a host cell or cell-free expression system containing the expression vector as described herein.

Also provided herein is a method of producing a recombinant antibody or antigen binding fragment as described herein, the method comprising culturing the host cell or cell free expression system as described herein under conditions which permit expression of the antibody or antigen binding fragment and recovering the expressed antibody or antigen binding fragment.

In another aspect of the invention, provided herein is a pharmaceutical composition comprising an antibody or antigen binding fragment as described herein, and at least one pharmaceutically acceptable carrier or excipient.

The antibody or antigen binding fragment as described herein, or the pharmaceutical composition as described herein are in a further aspect for use as a medicament. In a further aspect, there is provided a method of treating a subject in need thereof, wherein the method comprises administering to the subject a therapeutically effective amount of an antibody or antigen binding fragment as described herein or a pharmaceutical composition as described herein.

The antibody, antigen binding fragment or pharmaceutical composition may be administered to prevent or treat a disease or condition associated with the presence or formation of galectin-10 crystals. Suitably, the disease or condition can be selected from the group consisting of: asthma; chronic rhinosinusitis; celiac disease; helminth infection; gastrointestinal eosinophilic inflammation; cystic fibrosis (CF); allergic bronchopulmonary aspergillosis (ABPA); Churg-Straus vasculitis; chronic eosinophilic pneumonia; and acute myeloid leukemia (AML). In a preferred embodiment, the disease or condition is asthma. In another preferred embodiment, the disease or condition is cystic fibrosis.

The invention also provides use of an antibody or antigen binding fragment as described herein for the detection of galectin-10 in a sample obtained from a patient. Suitably, the patient sample can be a mucus sample or a sputum sample.

The invention also provides a kit comprising an antibody or antigen binding fragment as described herein. The kit may further comprise instructions for use.

### BRIEF DESCRIPTION OF DRAWINGS

FIG. 1 shows the results of two duplicate experiments. They demonstrate how the mean Charcot Leyden Crystal (CLC) area changes as a function of time when incubated with humanised Fab clones g18C06, g18E04, g18G07, g18G12, g20H09 and g23H09. The dissolution of CLCs as a function of time was recorded. Clones were tested at a concentration of 250 μg/mL (n=4) in two independent experiments and images captured CLCs in the samples at 2, 5, 7, and 16 h after antibody addition. The images were segmented using an algorithm to detect individual crystals and the total crystal area per well was determined. In this assay, 1 μL of the antibody sample was diluted at 1.5 mg/ml and then incubated with recombinant CLCs formed with 1 μg of Gal10. The y-axis used is the mean percentage crystal area dissolved per well after the incubation with each of the antibody samples. Samples denoted “TO” correspond to the reference samples of each of the clones (samples that were stored at −80° C. prior to analysis). Samples denoted “T2W” were stored for 2 weeks at a temperature of 37° C. prior to analysis.

FIG. 2 shows a graph illustrating the rate of dissolution of recombinant Charcot Leyden Crystals (CLCs) by g7B07 and g24F02_N53A (hFab) as determined by spinning disc confocal microscopy. A sample containing only PBS was used as a negative control. FIG. 2A shows a schematic representation of the recombinant CLC dissolution assay; FIG. 2B shows the results of the assay. The initial area covered by the CLC at the beginning of the experiment was defined as 1 and the surface occupied by the CLC was determined using software. Samples denoted “TO” correspond to the reference samples of each of the clones (samples that were stored at −80° C. prior to analysis). Samples denoted “T2W” were stored for 2 weeks at a temperature of 37° C. prior to analysis.

FIG. 3 shows the protein concentration for clones g18C06, g20H09, g23H09, g24F02_N53A and g7B07 as determined by measuring the Absorbance (A) at a wavelength of 280 nm using a Nanodrop. For the avoidance of doubt, whilst the sample names in the figure do not contain the prefix “g”, the tested clones were all germlined clones. Samples denoted “T0” correspond to the reference samples of each of the clones (samples that were stored at −80° C. prior to analysis). Samples denoted “TxW+y° C.” were stored for x week(s) at a temperature of y° C. prior to analysis, for example samples denoted “T1W+5° C.” were stored at +5° C. for 1 week prior to analysis, samples denoted “T1W+25° C.” were stored at +25° C. for 1 week prior to analysis, and so forth. Samples denoted “1F/FT” were subjected to one freeze-thaw cycle prior to prior to analysis; samples denoted “10F/FT” were subjected to 10 freeze-thaw cycles prior to prior to analysis; and samples denoted “low pH” were subjected to a pH of 3.7 for 2 hours prior to analysis.

FIG. 4 shows the percentage relative activity for clones g18C06, g20H09, g23H09, g24F02_N53A and g7B07 as determined by surface plasmon reference (SPR). For the avoidance of doubt, whilst the sample names in the figure do not contain the prefix “g”, the tested clones were all germlined clones. Samples denoted “TO” correspond to the reference samples of each of the clones (samples that were stored at −80° C. prior to SPR analysis). Samples denoted “TxW+y° C.” were stored for x week(s) at a temperature of y° C. prior to SPR analysis, for example samples denoted “T1W+5° C.” were stored at +5° C. for 1 week prior to SPR analysis, samples denoted “T1W+25° C.” were stored at +25° C. for 1 week prior to SPR analysis, and so forth. Samples denoted “1F/FT” were subjected to one freeze-thaw cycle prior to prior to SPR analysis; samples denoted “10F/FT” were subjected to 10 freeze-thaw cycles prior to prior to SPR analysis; and samples denoted “low pH” were subjected to a pH of 3.7 for 2 hours prior to analysis.

FIG. 5 shows the percentage purity of clones g18C06, g20H09, g23H09, g24F02_N53A and g7B07 as determined by SE-HPLC. For the avoidance of doubt, whilst the sample names in the figure do not contain the prefix “g”, the tested clones were all germlined clones. The top graph shows the percentage monomer in each of the samples, the middle graph shows the percentage of total aggregates in each of the samples and the bottom graph shows the percentage of total fragments in each of the samples. Samples denoted “TO” correspond to the reference samples of each of the clones (samples that were stored at −80° C. prior to SE-HPLC). Samples denoted “TxW+y° C.” were stored for x week(s) at a temperature of y° C. prior to SE-HPLC analysis, for example samples denoted “T1W+5° C.” were stored at +5° C. for 1 week prior to SE-HPLC, samples denoted “T1W+25° C.” were stored at +25° C. for 1 week prior to SE-HPLC, and so forth. Samples denoted “1F/FT” were subjected to one freeze-thaw cycle prior to prior to SE-HPLC; samples denoted “10F/FT” were subjected to 10 freeze-thaw cycles prior to prior to SE-HPLC; and samples denoted “low pH” were subjected to a pH of 3.7 for 2 hours prior to analysis.

FIG. 6 shows the percentage purity of the clones as assessed by capillary gel electrophoresis (cGE). The clone samples were also subjected to various stresses prior to determination of purity to assess the impact of the stresses on sample purity. The top graph illustrates the percentage purity of the intact Fab under non-reducing conditions and the bottom graph illustrates the percentage of total Fab under reducing conditions. Samples denoted “TO” correspond to the reference samples of each of the clones (samples were stored at −80° C. prior to cGE); samples denoted “T4W+5° C.” were stored at +5° C. for 4 weeks prior to cGE; samples denoted “T4W+25° C.” were stored at +25° C. for 4 weeks prior to cGE; samples denoted “T4W+37° C.” were stored at +37° C. for 4 weeks prior to cGE; samples denoted “1F/FT” were subjected to one freeze-thaw cycle prior to prior to cGE; and samples denoted “10F/FT” were subjected to 10 freeze-thaw cycles prior to prior to cGE analysis. For the avoidance of doubt, whilst the sample names in the figure do not contain the prefix “g”, the tested clones were all germlined clones.

FIG. 7 shows that clones g18C06, g20H09 and g23H09 and g24F02_N53A dissolve GAL10 crystals at a comparable rate to clone g7B07_N53A. For the avoidance of doubt, whilst the sample names in the figure do not contain the prefix “g”, the tested clones were all germlined clones.

FIG. 8 shows that clones g18C06, g20H09 and g23H09 are able to dissolve GAL10 crystals. The clones were able to dissolve GAL10 crystals after being stored under different conditions for 4 weeks and after nebulization. Further details of the assay performed can be found in the examples section entitled “Materials and Protocols used in examples 1-4” (see Assay 1 described therein). For the avoidance of doubt, whilst the sample names in the figure do not contain the prefix “g”, the tested clones were all germlined clones.

FIG. 9 demonstrates that clones g23H09 and g24F02_N53A dissolve GAL10 crystals of different sizes. The clones were able to dissolve GAL10 crystals after storage at 5° C. for 4 weeks and after nebulization (samples annotated “solo 0125”). Further details of the assay performed can be found in the examples section entitled “Materials and Protocols used in examples 1-4” (see Assay 2 described therein).

FIG. 10 shows the global DR beta 1 (DRB1) risk scores for 44 marketed therapeutic antibodies as well as the risk scores for clones g20H09, g23H09, g18C06 and g24F02_N53A. Human antibodies are shown by mid-grey bars, humanized by grey bars and chimeric by dark grey bars.

FIG. 11 shows the percentage of donors with an IFNγ (left graphs) and an IL-5 (right graphs) response to clones g20H09, g23H09, g18C06 and g24F02_N53A. A distribution-free resampling (DFR2x) algorithm was used for statistical analysis as well as DFReq (Moodie et al. Cancer Immunol Immunother 59, 1489-1501 (2010). The KLH sample was used as a positive control.

FIG. 12 shows the IFNγ (left pane) and IL-5 (right pane) responses in a donor test population (n=31) of clones g20H09, g23H09, g18C06 and g24F02_N53A (A DFR2x algorithm was used for statistical analysis (Moodie et al. Cancer Immunol Immunother 59, 1489-1501 (2010)).

### DETAILED DESCRIPTION

### A. Definitions

Unless defined otherwise, all technical and scientific terms used herein have the same meaning as is commonly understood by one skilled in the art in the technical field of the invention.

“Antibody” or “Immunoglobulin”—As used herein, the term “immunoglobulin” includes a polypeptide having a combination of two heavy and two light chains whether or not it possesses any relevant specific immunoreactivity. “Antibodies” refer to such assemblies which have significant known specific immunoreactive activity to an antigen of interest (herein galectin-10). The term “galectin-10 antibodies” is used herein to refer to antibodies which exhibit immunological specificity for the galectin-10 protein, including human galectin-10, and in some cases species homologues thereof. Antibodies and immunoglobulins comprise light and heavy chains, with or without an interchain covalent linkage between them. Basic immunoglobulin structures in vertebrate systems are relatively well understood.

The generic term “immunoglobulin” comprises five distinct classes of antibody that can be distinguished biochemically. All five classes of antibodies are within the scope of the present invention. The following discussion will generally be directed to the IgG class of immunoglobulin molecules. With regard to IgG, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

The light chains of an antibody are classified as either kappa or lambda (κ,λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA, IgD or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant invention.

As indicated above, the variable region of an antibody allows the antibody to selectively recognize and specifically bind epitopes on antigens. That is, the VL domain and VH domain of an antibody combine to form the variable region that defines a three dimensional antigen binding site. This quaternary antibody structure forms the antigen binding site present at the end of each arm of the Y. More specifically, the antigen binding site is defined by three complementary determining regions (CDRs) on each of the VH and VL chains.

“Galectin-10”—As used herein, the term “galectin-10” (or Gal10 or Gal-10, which are used interchangeably herein) refers to the small, hydrophobic glycan binding protein that autocrystallizes to form Charcot-Leyden crystals. Galectin-10 is also referred to as Charcot-Leyden crystal protein (CLCP), eosinophil lysophospholipase and lysolecithin acylhydrolase. The term “galectin-10” is broad enough to cover the human protein and any species homologues. The amino acid sequence of the full-length human galectin-10 is represented by SEQ ID NO: 25 (see below). This sequence corresponds to the sequence deposited in the UniProt database as human galectin-10, accession number Q05315. Also encompassed within the term “galectin-10” are naturally occurring variants of the human sequence, for example the Ala→Val variant at position 28.

```
SEQ ID NO: 25
1 10 20 30 40 50
MSLLPVPYTE AASLSTGSTV TIKGRPLACF LNEPYLQVDF HTEMKEESDI
                60 70 80 90 100
VFHFQVCFGR RVVMNSREYG AWKQQVESKN MPFQDGQEFE LSISVLPDKY
              110 120 130 140
QVMVNGQSSY TFDHRIKPEA VKMVQVWRDI SLTKFNVSYL KR
```

“Galectin-10 crystals” or “Charcot-Leyden crystals”—the terms “galectin-10 crystals”, “Charcot-Leyden crystals” and “CLCs” are used herein interchangeably to refer to crystals formed of galectin-10. The crystals formed by galectin-10 are typically bi-pyramidal hexagonal crystals and are approximately 20-40 μm in length and approximately 2-4 μm width. These crystals have been associated with eosinophilic inflammatory disorders.

“Epitope”—As used herein, the term “epitope” means the region of the galectin-10 protein to which the antagonist binds. An antagonist will typically bind to its respective galectin-10 epitope via a complementary binding site on the antagonist. The epitope to which the antagonist binds will typically comprise one or more amino acids from the full-length galectin-10 protein. The epitope may include amino acids that are contiguous in the galectin-10 protein i.e. a linear epitope or may include amino acids that are non-contiguous in the galectin-10 protein i.e. a conformational epitope.

“Binding Site”—As used herein, the term “binding site” comprises a region of a polypeptide which is responsible for selectively binding to a target antigen of interest (e.g. galectin-10). Binding domains comprise at least one binding site. Exemplary binding domains include an antibody variable domain. The antibody molecules of the invention may comprise a single binding site or multiple (e.g., two, three or four) binding sites.

“Derived From”—As used herein the term “derived from” a designated protein (e.g. a camelid antibody or antigen binding fragment thereof) refers to the origin of the polypeptide or amino acid sequence. In one embodiment, the polypeptide or amino acid sequence which is derived from a particular starting polypeptide is a CDR sequence or sequence related thereto. In one embodiment, the amino acid sequence which is derived from a particular starting polypeptide is not contiguous. For example, in one embodiment, one, two, three, four, five, or six CDRs are derived from a starting antibody. In one embodiment, the polypeptide or amino acid sequence which is derived from a particular starting polypeptide or amino acid sequence has an amino acid sequence that is essentially identical to that of the starting sequence, or a portion thereof wherein the portion consists of at least 3-5 amino acids, at least 5-10 amino acids, at least 10-20 amino acids, at least 20-30 amino acids, or at least 30-50 amino acids, or which is otherwise identifiable to one of ordinary skill in the art as having its origin in the starting sequence. In one embodiment, the one or more CDR sequences derived from the starting antibody are altered to produce variant CDR sequences, e.g. affinity variants, wherein the variant CDR sequences maintain target antigen binding activity.

“Camelid-Derived”—In certain preferred embodiments, the antibodies of the invention comprise framework amino acid sequences and/or CDR amino acid sequences derived from a camelid conventional antibody or a VHH antibody raised by active immunisation of a camelid. However, antibodies of the invention comprising camelid-derived amino acid sequences may be engineered to comprise framework and/or constant region sequences derived from a human amino acid sequence (i.e. a human antibody) or other non-camelid mammalian species. For example, a human or non-human primate framework region, heavy chain portion, and/or hinge portion may be included in the galectin-10 antibodies. In one embodiment, one or more non-camelid amino acids may be present in the framework region of a “camelid-derived” antibody, e.g., a camelid framework amino acid sequence may comprise one or more amino acid mutations in which the corresponding human or non-human primate amino acid residue is present. Moreover, camelid-derived VH and VL domains, or humanised variants thereof, may be linked to the constant domains of human antibodies to produce a chimeric molecule, as described elsewhere herein.

“VHH antibodies”—As used herein the term “VHH antibody” or “heavy-chain only antibody” refers to a type of antibody produced only by species of the Camelidae family, which includes camels, llama, alpaca. Heavy chain-only antibodies or VHH antibodies are composed of two heavy chains and are devoid of light chains. Each heavy chain has a variable domain at the N-terminus, and these variable domains are referred to as “VHH” domains in order to distinguish them from the variable domains of the heavy chains of the conventional heterotetrameric antibodies i.e. the VH domains, described above.

“Conservative amino acid substitution”—A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide may be replaced with another amino acid residue from the same side chain family. In another embodiment, a string of amino acids can be replaced with a structurally similar string that differs in order and/or composition of side chain family members.

“Heavy chain portion”—As used herein, the term “heavy chain portion” includes amino acid sequences derived from the constant domains of an immunoglobulin heavy chain. A polypeptide comprising a heavy chain portion comprises at least one of: a CH1 domain, a hinge (e.g., upper, middle, and/or lower hinge region) domain, a CH2 domain, a CH3 domain, or a variant or fragment thereof. In one embodiment, an antibody or antigen binding fragment of the invention may comprise the Fc portion of an immunoglobulin heavy chain (e.g., a hinge portion, a CH2 domain, and a CH3 domain). In another embodiment, an antibody or antigen binding fragment of the invention may lack at least a portion of a constant domain (e.g., all or part of a CH2 domain). In certain embodiments, at least one, and preferably all, of the constant domains are derived from a human immunoglobulin heavy chain. For example, in one preferred embodiment, the heavy chain portion comprises a fully human hinge domain. In other preferred embodiments, the heavy chain portion comprises a fully human Fc portion (e.g., hinge, CH2 and CH3 domain sequences from a human immunoglobulin).

In certain embodiments, the constituent constant domains of the heavy chain portion are from different immunoglobulin molecules. For example, a heavy chain portion of a polypeptide may comprise a CH2 domain derived from an IgG1 molecule and a hinge region derived from an IgG3 or IgG4 molecule. In other embodiments, the constant domains are chimeric domains comprising portions of different immunoglobulin molecules. For example, a hinge may comprise a first portion from an IgG1 molecule and a second portion from an IgG3 or IgG4 molecule. As set forth above, it will be understood by one of ordinary skill in the art that the constant domains of the heavy chain portion may be modified such that they vary in amino acid sequence from the naturally occurring (wild-type) immunoglobulin molecule. That is, the polypeptides of the invention disclosed herein may comprise alterations or modifications to one or more of the heavy chain constant domains (CH1, hinge, CH2 or CH3) and/or to the light chain constant region domain (CL). Exemplary modifications include additions, deletions or substitutions of one or more amino acids in one or more domains.

“Chimeric”—A “chimeric” protein comprises a first amino acid sequence linked to a second amino acid sequence with which it is not naturally linked in nature. The amino acid sequences may normally exist in separate proteins that are brought together in the fusion polypeptide or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship. Exemplary chimeric antibodies of the invention include fusion proteins comprising camelid-derived VH and VL domains, or humanised variants thereof, fused to the constant domains of a human antibody, e.g. human IgG1, IgG2, IgG3 or IgG4.

“Variable region” or “variable domain”—The terms “variable region” and “variable domain” are used herein interchangeably and are intended to have equivalent meaning. The term “variable” refers to the fact that certain portions of the variable domains VH and VL differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the VL domain and the VH domain which form part of the antigen binding site. The first, second and third hypervariable loops of the VLambda light chain domain are referred to herein as L1(λ), L2(λ) and L3(λ) and may be defined as comprising residues 24-33 (L1(λ), consisting of 9, 10 or 11 amino acid residues), 49-53 (L2(λ), consisting of 3 residues) and 90-96 (L3(λ), consisting of 5 residues) in the VL domain (Morea et al., Methods 20:267-279 (2000)). The first, second and third hypervariable loops of the VKappa light chain domain are referred to herein as L1(κ), L2(κ) and L3(κ) and may be defined as comprising residues 25-33 (L1(κ), consisting of 6, 7, 8, 11, 12 or 13 residues), 49-53 (L2(κ), consisting of 3 residues) and 90-97 (L3(κ), consisting of 6 residues) in the VL domain (Morea et al., Methods 20:267-279 (2000)). The first, second and third hypervariable loops of the VH domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the VH domain (Morea et al., Methods 20:267-279 (2000)).

Unless otherwise indicated, the terms L1, L2 and L3 respectively refer to the first, second and third hypervariable loops of a VL domain, and encompass hypervariable loops obtained from both Vkappa and Vlambda isotypes. The terms H1, H2 and H3 respectively refer to the first, second and third hypervariable loops of the VH domain, and encompass hypervariable loops obtained from any of the known heavy chain isotypes, including γ, ε, δ, α or μ.

The hypervariable loops L1, L2, L3, H1, H2 and H3 may each comprise part of a “complementarity determining region” or “CDR”, as defined below. The terms “hypervariable loop” and “complementarity determining region” are not strictly synonymous, since the hypervariable loops (HVs) are defined on the basis of structure, whereas complementarity determining regions (CDRs) are defined based on sequence variability (Kabat et al., Sequences of Proteins of Immunological Interest, 5th Ed. Public Health Service, National Institutes of Health, Bethesda, MD., 1983) and the limits of the HVs and the CDRs may be different in some VH and VL domains.

The CDRs of the VL and VH domains can typically be defined as comprising the following amino acids: residues 24-34 (LCDR1), 50-56 (LCDR2) and 89-97 (LCDR3) in the light chain variable domain, and residues 31-35 or 31-35b (HCDR1), 50-65 (HCDR2) and 95-102 (HCDR3) in the heavy chain variable domain; (Kabat et al., Sequences of Proteins of Immunological Interest, 5th Ed. Public Health Service, National Institutes of Health, Bethesda, MD. (1991)). Thus, the HVs may be comprised within the corresponding CDRs and references herein to the “hypervariable loops” of VH and VL domains should be interpreted as also encompassing the corresponding CDRs, and vice versa, unless otherwise indicated.

The more highly conserved portions of variable domains are called the framework region (FR), as defined below. The variable domains of native heavy and light chains each comprise four FRs (FR1, FR2, FR3 and FR4, respectively), largely adopting a β-sheet configuration, connected by the three hypervariable loops. The hypervariable loops in each chain are held together in close proximity by the FRs and, with the hypervariable loops from the other chain, contribute to the formation of the antigen binding site of antibodies. Structural analysis of antibodies revealed the relationship between the sequence and the shape of the binding site formed by the complementarity determining regions (Chothia et al., J. Mol. Biol. 227:799-817 (1992)); Tramontano et al., J. Mol. Biol, 215:175-182 (1990)).

“CDR”—As used herein, the term “CDR” or “complementarity determining region” means the non-contiguous antigen binding sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat based on sequence comparisons.

TABLE 1
CDR definitions

| | CDR Definitions | | |
|---|---|---|---|
| | Kabat¹ | Chothia² | MacCallum³ |
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

“Framework region”—The term “framework region” or “FR region” as used herein, includes the amino acid residues that are part of the variable region, but are not part of the CDRs (e.g., using the Kabat definition of CDRs). Therefore, a variable region framework is between about 100-120 amino acids in length but includes only those amino acids outside of the CDRs. For the specific example of a heavy chain variable domain and for the CDRs as defined by Kabat et al., framework region 1 corresponds to the domain of the variable region encompassing amino acids 1-30; framework region 2 corresponds to the domain of the variable region encompassing amino acids 36-49; framework region 3 corresponds to the domain of the variable region encompassing amino acids 66-94, and framework region 4 corresponds to the domain of the variable region from amino acids 103 to the end of the variable region. The framework regions for the light chain are similarly separated by each of the light chain variable region CDRs. Similarly, using the definition of CDRs by Chothia et al. or McCallum et al. the framework region boundaries are separated by the respective CDR termini as described above. In preferred embodiments the CDRs are as defined by Kabat.

In naturally occurring antibodies, the six CDRs present on each monomeric antibody are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

“Hinge region”—As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux K. H. et al. J. Immunol. 161:4083-90 1998). Antibodies of the invention comprising a “fully human” hinge region may contain one of the hinge region sequences shown in Table 2 below.

TABLE 2
Human hinge sequences

| IgG | Upper hinge | Middle hinge | Lower hinge |
|---|---|---|---|
| IgG1 | EPKSCDKTHT | CPPCP | APELLGGP |
| | (SEQ ID NO: 26) | (SEQ ID NO: 27) | (SEQ ID NO: 28) |
| IgG3 | ELKTPLGDTTHT | CPRCP(EPKSCDTPPPCPRCP)₃ | APELLGGP |
| | (SEQ ID NO: 29) | (SEQ ID NO: 30) | (SEQ ID NO: 31) |
| IgG4 | ESKYGPP | CPSCP | APEFLGGP |
| | (SEQ ID NO: 32) | (SEQ ID NO: 33) | (SEQ ID NO: 34) |
| IgG42 | ERK | CCVECPPPCP | APPVAGP |
| | (SEQ ID NO: 35) | (SEQ ID NO: 36) | (SEQ ID NO: 37) |

“CH2 domain”—As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system, Kabat E A et al. Sequences of Proteins of Immunological Interest. Bethesda, US Department of Health and Human Services, NIH. 1991). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

“Fragment”—The term “fragment”, as used in the context of antibodies of the invention, refers to a part or portion of an antibody or antibody chain comprising fewer amino acid residues than an intact or complete antibody or antibody chain. The term “antigen binding fragment” refers to a polypeptide fragment of an immunoglobulin or antibody that binds the antigen or competes with intact antibody (i.e., with the intact antibody from which they were derived) for antigen binding (i.e., specific binding to galectin-10). As used herein, the term “fragment” of an antibody molecule includes antigen binding fragments of antibodies, for example, an antibody light chain variable domain (VL), an antibody heavy chain variable domain (VH), a single chain antibody (scFv), a F(ab′) 2 fragment, a Fab fragment, an Fd fragment, an Fv fragment, a one-armed (monovalent) antibody, diabodies, triabodies, tetrabodies or any antigen binding molecule formed by combination, assembly or conjugation of such antigen binding fragments. The term “antigen binding fragment” as used herein is further intended to encompass antibody fragments selected from the group consisting of unibodies, domain antibodies and nanobodies. Fragments can be obtained, e.g., via chemical or enzymatic treatment of an intact or complete antibody or antibody chain or by recombinant means.

“Fab”—A “Fab” or “Fab fragment” refers to a molecule composed of a heavy chain and light chain wherein the light chain consists of the VL domain and the one constant domain (CL, C_{κ} or C_{λ}) and the heavy chain consists of the VH domain and the CH1 domain only. A Fab fragment is typically one arm of a Y-shaped immunoglobulin molecule. A Fab fragment can be generated from an immunoglobulin molecule by the action of the enzyme papain. Papain cleaves immunoglobulin molecules in the region of the hinge so as yield two Fab fragments and a separate Fc region.

“scFv” or “scFv fragment”—An scFv or scFv fragment means a single chain variable fragment. An scFv is a fusion protein of a VH domain and a VL domain of an antibody connected via a linker.

“Valency”—As used herein the term “valency” refers to the number of potential target binding sites in a polypeptide. Each target binding site specifically binds one target molecule or specific site on a target molecule. When a polypeptide comprises more than one target binding site, each target binding site may specifically bind the same or different molecules (e.g., may bind to different ligands or different antigens, or different epitopes on the same antigen).

“Specificity”—The term “specificity” refers to the ability to bind (e.g., immunoreact with) a given target, e.g. galectin-10. A polypeptide may be monospecific and contain one or more binding sites which specifically bind a target or a polypeptide may be multispecific and contain two or more binding sites which specifically bind the same or different targets.

“Synthetic”—As used herein the term “synthetic” with respect to polypeptides includes polypeptides which comprise an amino acid sequence that is not naturally occurring. For example, non-naturally occurring polypeptides which are modified forms of naturally occurring polypeptides (e.g., comprising a mutation such as an addition, substitution or deletion) or which comprise a first amino acid sequence (which may or may not be naturally occurring) that is linked in a linear sequence of amino acids to a second amino acid sequence (which may or may not be naturally occurring) to which it is not naturally linked in nature.

“Engineered”—As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques). Preferably, the antibodies of the invention are engineered, including for example, humanized and/or chimeric antibodies, and antibodies which have been engineered to improve one or more properties, such as antigen binding, stability/half-life, immunogenicty or effector function.

“Modified antibody”—As used herein, the term “modified antibody” includes synthetic forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen); heavy chain molecules joined to scFv molecules and the like. scFv molecules are known in the art and are described, e.g., in US U.S. Pat. No. 5,892,019. In addition, the term “modified antibody” includes multivalent forms of antibodies (e.g., trivalent, tetravalent, etc., antibodies that bind to three or more copies of the same antigen). In another embodiment, a modified antibody of the invention is a fusion protein comprising at least one heavy chain portion lacking a CH2 domain and comprising a binding domain of a polypeptide comprising the binding portion of one member of a receptor ligand pair.

The term “modified antibody” may also be used herein to refer to amino acid sequence variants of the antibodies of the invention as structurally defined herein. It will be understood by one of ordinary skill in the art that an antibody may be modified to produce a variant antibody which varies in amino acid sequence in comparison to the antibody from which it was derived. For example, nucleotide or amino acid substitutions leading to conservative substitutions or changes at “non-essential” amino acid residues may be made (e.g., in CDR and/or framework residues). Amino acid substitutions can include replacement of one or more amino acids with a naturally occurring or non-natural amino acid.

“Humanising substitutions”—As used herein, the term “humanising substitutions” refers to amino acid substitutions in which the amino acid residue present at a particular position in the VH or VL domain of an antibody (for example a camelid-derived galectin-10 antibody) is replaced with an amino acid residue which occurs at an equivalent position in a reference human VH or VL domain. The reference human VH or VL domain may be a VH or VL domain encoded by the human germline. Humanising substitutions may be made in the framework regions and/or the CDRs of the antibodies, defined herein.

“Humanised variants”—As used herein the term “humanised variant” refers to a variant antibody which contains one or more “humanising substitutions” compared to a reference antibody, wherein a portion of the reference antibody (e.g. the VH domain and/or the VL domain or parts thereof containing at least one CDR) has an amino acid derived from a non-human species, and the “humanising substitutions” occur within the amino acid sequence derived from a non-human species.

“Germlined variants”—The term “germlined variant” is used herein to refer specifically to “humanised variants” in which the “humanising substitutions” result in replacement of one or more amino acid residues present at a particular position(s) in the VH or VL domain of an antibody (for example a camelid-derived galectin-10 antibody) with an amino acid residue which occurs at an equivalent position in a reference human VH or VL domain encoded by the human germline. It is typical that for any given “germlined variant”, the replacement amino acid residues substituted into the germlined variant are taken exclusively, or predominantly, from a single human germline-encoded VH or VL domain. The terms “humanised variant” and “germlined variant” are often used interchangeably herein. Introduction of one or more “humanising substitutions” into a camelid-derived (e.g. llama derived) VH or VL domain results in production of a “humanised variant” of the camelid (llama)-derived VH or VL domain. If the amino acid residues substituted in are derived predominantly or exclusively from a single human germline-encoded VH or VL domain sequence, then the result may be a “human germlined variant” of the camelid (llama)-derived VH or VL domain.

“% identity”—As used herein is herein to describe the sequence similarity between two sequences, such as amino acid and nucleotide sequences. This may be determined by comparing the two sequences aligned in an optimum manner and in which the amino acid sequence to be compared can comprise additions or deletions with respect to the reference sequence for an optimum alignment between these two sequences. The percentage of identity is calculated by determining the number of identical positions for which the residue is identical between the two sequences, dividing this number of identical positions by the total number of positions in the comparison window and multiplying the result obtained by 100 in order to obtain the percentage of identity between these two sequences. For example, it is possible to use the BLAST program, “BLAST 2 sequences” (Tatusova et al, “Blast 2 sequences—a new tool for comparing protein and nucleotide sequences”, FEMS Microbiol Lett. 174:247-250) available on the site http://www.ncbi.nlm.nih.gov/gorf/bl2.html, the parameters used being those given by default (in particular for the parameters “open gap penalty”: 5, and “extension gap penalty”: 2; the matrix chosen being, for example, the matrix “BLOSUM 62” proposed by the program), the percentage of identity between the two sequences to be compared being calculated directly by the program.

“Affinity variants”—As used herein, the term “affinity variant” refers to a variant antibody which exhibits one or more changes in amino acid sequence compared to a reference antibody, wherein the affinity variant exhibits an altered affinity for the target antigen in comparison to the reference antibody. For example, affinity variants will exhibit a changed affinity for galectin-10, as compared to the reference galectin-10 antibody. Preferably the affinity variant will exhibit improved affinity for the target antigen, e.g. galectin-10, as compared to the reference antibody. Affinity variants typically exhibit one or more changes in amino acid sequence in the CDRs, as compared to the reference antibody. Such substitutions may result in replacement of the original amino acid present at a given position in the CDRs with a different amino acid residue, which may be a naturally occurring amino acid residue or a non-naturally occurring amino acid residue. The amino acid substitutions may be conservative or non-conservative.

“High human homology”—An antibody comprising a heavy chain variable domain (VH) and a light chain variable domain (VL) may be considered as having high human homology if the VH domains and the VL domains, taken together, exhibit at least 90% amino acid sequence identity to the closest matching human germline VH and VL sequences. Antibodies having high human homology may include antibodies comprising VH and VL domains of native non-human antibodies which exhibit sufficiently high % sequence identity to human germline sequences, including for example antibodies comprising VH and VL domains of camelid conventional antibodies, as well as engineered, especially humanised or germlined, variants of such antibodies and also “fully human” antibodies.

In one embodiment the VH domain of the antibody with high human homology may exhibit an amino acid sequence identity or sequence homology of 80% or greater with one or more human VH domains across the framework regions FR1, FR2, FR3 and FR4. In other embodiments the amino acid sequence identity or sequence homology between the VH domain of the polypeptide of the invention and the closest matching human germline VH domain sequence may be 85% or greater, 90% or greater, 95% or greater, 97% or greater, or up to 99% or even 100%.

In one embodiment the VH domain of the antibody with high human homology may contain one or more (e.g. 1 to 10) amino acid sequence mis-matches across the framework regions FR1, FR2, FR3 and FR4, in comparison to the closest matched human VH sequence.

In another embodiment the VL domain of the antibody with high human homology may exhibit a sequence identity or sequence homology of 80% or greater with one or more human VL domains across the framework regions FR1, FR2, FR3 and FR4. In other embodiments the amino acid sequence identity or sequence homology between the VL domain of the polypeptide of the invention and the closest matching human germline VL domain sequence may be 85% or greater 90% or greater, 95% or greater, 97% or greater, or up to 99% or even 100%.

In one embodiment the VL domain of the antibody with high human homology may contain one or more (e.g. 1 to 10) amino acid sequence mis-matches across the framework regions FR1, FR2, FR3 and FR4, in comparison to the closest matched human VL sequence.

### B. Antibodies and Antigen Binding Fragments that Bind to Galectin-10

As described above, the present invention is directed to antibodies or antigen binding fragments that bind to galectin-10. The term “antibody” is used in the broadest sense and encompasses, but is not limited to, monoclonal antibodies (including full length monoclonal antibodies), polyclonal antibodies, multispecific antibodies (i.e., bispecific antibodies), so long as they exhibit the appropriate immunological specificity for the galectin-10 protein. The antibodies and antigen binding fragments that bind to galectin-10 described herein may exhibit immunological specificity for any galectin-10 epitope.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes) on the antigen, each monoclonal antibody is directed against a single determinant or epitope on the antigen. “Antibody fragments” or “antigen binding fragments” comprise a portion of a full length antibody, generally the antigen binding or variable domain thereof. Antibody fragments are described elsewhere herein and examples of antibody fragments include Fab, Fab′, F(ab′)2, bi-specific Fab's, and Fv fragments, diabodies, linear antibodies, single-chain antibody molecules, a single chain variable fragment (scFv) and multispecific antibodies formed from antibody fragments (see Holliger and Hudson, Nature Biotechnol. 23:1126-36 (2005), the contents of which are incorporated herein by reference).

The antibodies and antigen binding fragments that bind galectin-10 described herein are intended for human therapeutic use and therefore, will typically be immunoglobulins of the IgA, IgD, IgE, IgG, IgM type, often of the IgG type, in which case they can belong to any of the four sub-classes IgG1, IgG2a and b, IgG3 or IgG4. In preferred embodiments, the antibodies are IgG antibodies. Monoclonal antibodies are preferred since they are highly specific, being directed against a single antigenic site. In certain preferred embodiments, the antigen binding fragments that bind galectin-10 are Fab fragments or “Fabs”.

The antibodies and antigen binding fragments that bind galectin-10 may exhibit high human homology as defined elsewhere herein. Such antibody molecules having high human homology may include antibodies comprising VH and VL domains of native non-human antibodies which exhibit sufficiently high percentage sequence identity to human germline sequences. In certain embodiments, the antibodies or antigen binding fragments thereof are humanised or germlined variants of non-human antibodies.

In certain embodiments, the antibodies and antigen binding fragments that bind galctin-10 as described herein may be camelid-derived. Camelid-derived antibodies may be heavy-chain only antibodies i.e. VHH antibodies or may be conventional heterotetrameric antibodies. In preferred embodiments, the galectin-10 antibodies and antigen binding fragments are derived from camelid heterotetrameric antibodies. In further preferred embodiments, the galectin-10 antibodies are derived from VHH antibodies.

For example, the antibodies and antigen binding fragments as described herein may be selected from immune libraries obtained by a method comprising the step of immunizing a camelid with the target of interest i.e. galectin-10. The camelid may be immunized with the target protein or polypeptide fragment thereof, or with an mRNA molecule or cDNA molecule expressing the protein or a polypeptide fragment thereof. Methods for producing antibodies in camelid species and selecting antibodies against preferred targets from camelid immune libraries are described in, for example, International patent application no. WO2010/001251, incorporated herein by reference.

In certain embodiments, the antibodies and antigen binding fragments may be camelid-derived in that they comprise at least one hypervariable (HV) loop or complementarity determining region (CDR) obtained from a VH domain or a VL domain of a species in the family Camelidae. In particular, the antibodies and antigen binding fragments may comprise VH and/or VL domains, or CDRs thereof, obtained by active immunisation of outbred camelids, i.e. llamas, with galectin-10.

The term “obtained from” in this context implies a structural relationship, in the sense that the HVs or CDRs of the antibodies embody an amino acid sequence (or minor variants thereof) which was originally encoded by a Camelidae immunoglobulin gene. However, this does not necessarily imply a particular relationship in terms of the production process used to prepare the antibodies or antigen binding fragments thereof.

Camelid-derived antibodies or antigen binding fragments thereof may be derived from any camelid species, including inter alia, llama, dromedary, alpaca, vicuna, guanaco or camel.

Antibody molecules comprising camelid-derived VH and VL domains, or CDRs thereof, are typically recombinantly expressed polypeptides, and may be chimeric polypeptides. The term “chimeric polypeptide” refers to an artificial (non-naturally occurring) polypeptide which is created by juxtaposition of two or more peptide fragments which do not otherwise occur contiguously. Included within this definition are “species” chimeric polypeptides created by juxtaposition of peptide fragments encoded by two or more species, i.e. camelid and human.

In certain embodiments, the entire VH domain and/or the entire VL domain may be obtained from a species in the family Camelidae. The camelid-derived VH domain and/or the camelid-derived VL domain may then be subject to protein engineering, in which one or more amino acid substitutions, insertions or deletions are introduced into the camelid amino acid sequence.

These engineered changes preferably include amino acid substitutions relative to the camelid sequence. Such changes include “humanisation” or “germlining” wherein one or more amino acid residues in a camelid-encoded VH or VL domain are replaced with equivalent residues from a homologous human-encoded VH or VL domain.

Isolated camelid VH and VL domains obtained by active immunisation of a camelid (i.e. llama) with galectin-10 can be used as a basis for engineering antibodies and antigen binding fragments that bind galectin-10 in accordance with the present invention. Starting from intact camelid VH and VL domains, it is possible to engineer one or more amino acid substitutions, insertions or deletions which depart from the starting camelid sequence. In certain embodiments, such substitutions, insertions or deletions may be present in the framework regions of the VH domain and/or the VL domain.

In other embodiments, there are provided “chimeric” antibody molecules comprising camelid-derived VH and VL domains (or engineered variants thereof) and one or more constant domains from a non-camelid antibody, for example human-encoded constant domains (or engineered variants thereof). In such embodiments it is preferred that both the VH domain and the VL domain are obtained from the same species of camelid, for example both VH and VL may be from Lama glama or both VH and VL may be from Lama pacos (prior to introduction of engineered amino acid sequence variation). In such embodiments both the VH and the VL domain may be derived from a single animal, particularly a single animal which has been actively immunised with the antigen of interest.

As an alternative to engineering changes in the primary amino acid sequence of Camelidae VH and/or VL domains, individual camelid-derived hypervariable loops or CDRs, or combinations thereof, can be isolated from camelid VH/VL domains and transferred to an alternative (i.e. non-Camelidae) framework, e.g. a human VH/VL framework, by CDR grafting.

In non-limiting embodiments, the antibodies described herein may comprise CH1 domains and/or CL domains (from the heavy chain and light chain, respectively), the amino acid sequence of which is fully or substantially human. For antibody molecules intended for human therapeutic use, it is typical for the entire constant region of the antibody, or at least a part thereof, to have fully or substantially human amino acid sequence. Therefore, one or more or any combination of the CH1 domain, hinge region, CH2 domain, CH3 domain and CL domain (and CH4 domain if present) may be fully or substantially human with respect to its amino acid sequence. The CH1 domain, hinge region, CH2 domain, CH3 domain and/or CL domain (and/or CH4 domain if present) may be derived from a human antibody, preferably a human IgG antibody, more preferably a human IgG1 antibody of subtype IgG1, IgG2, IgG3 or IgG4.

Advantageously, the CH1 domain, hinge region, CH2 domain, CH3 domain and CL domain (and CH4 domain if present) may all have fully or substantially human amino acid sequence. In the context of the constant region of a humanised or chimeric antibody, or an antibody fragment, the term “substantially human” refers to an amino acid sequence identity of at least 90%, or at least 92%, or at least 95%, or at least 97%, or at least 99% with a human constant region. The term “human amino acid sequence” in this context refers to an amino acid sequence which is encoded by a human immunoglobulin gene, which includes germline, rearranged and somatically mutated genes. The invention also contemplates polypeptides comprising constant domains of “human” sequence which have been altered, by one or more amino acid additions, deletions or substitutions with respect to the human sequence, excepting those embodiments where the presence of a “fully human” hinge region is expressly required.

The antibodies that bind galectin-10 may have one or more amino acid substitutions, insertions or deletions within the constant region of the heavy and/or the light chain, particularly within the Fc region. Amino acid substitutions may result in replacement of the substituted amino acid with a different naturally occurring amino acid, or with a non-natural or modified amino acid. Other structural modifications are also permitted, such as for example changes in glycosylation pattern (e.g. by addition or deletion of N- or O-linked glycosylation sites).

The antibodies may be modified within the Fc region to increase binding affinity for the neonatal receptor FcRn. The increased binding affinity may be measurable at acidic pH (for example from about approximately pH 5.5 to approximately pH 6.0). The increased binding affinity may also be measurable at neutral pH (for example from approximately pH 6.9 to approximately pH 7.4). By “increased binding affinity” is meant increased binding affinity to FcRn relative to the unmodified Fc region. Typically the unmodified Fc region will possess the wild-type amino acid sequence of human IgG1, IgG2, IgG3 or IgG4. In such embodiments, the increased FcRn binding affinity of the antibody molecule having the modified Fc region will be measured relative to the binding affinity of wild-type IgG1, IgG2, IgG3 or IgG4 for FcRn.

In certain embodiments, one or more amino acid residues within the Fc region may be substituted with a different amino acid so as to increase binding to FcRn. Several Fc substitutions have been reported that increase FcRn binding and thereby improve antibody pharmacokinetics. Such substitutions are reported in, for example, Zalevsky et al. (2010) Nat. Biotechnol. 28 (2): 157-9; Hinton et al. (2006) J Immunol. 176:346-356; Yeung et al. (2009) J Immunol. 182:7663-7671; Presta LG. (2008) Curr. Op. Immunol. 20:460-470; and Vaccaro et al. (2005) Nat. Biotechnol. 23 (10): 1283-88, the contents of which are incorporated herein in their entirety.

In certain embodiments, the antibodies comprise a modified human IgG Fc domain comprising or consisting of the amino acid substitutions H433K and N434F, wherein the Fc domain numbering is in accordance with EU numbering (Edelman, G. M. et al., Proc. Natl. Acad. USA, 63, 78-85 (1969) and Kabat, E. A.; National Institutes of Health (U.S.) Office of the Director. Sequences of Proteins of Immunological Interest, 5th ed.; DIANE Publishing: Collingdale, PA, USA, (1991)). In a further embodiment, the antibodies described herein comprise a modified human IgG Fc domain comprising or consisting of the amino acid substitutions M252Y, S254T, T256E, H433K and N434F, wherein the Fc domain numbering is in accordance with EU numbering. In preferred embodiments, the present invention provides antibodies that bind to galectin-10 (i.e. anti-galectin-10 antibodies) wherein the antibodies comprise at least one variant Fc domain incorporating ABDEG™ technology. ABDEG™ antibodies and FcRn antagonists incorporating ABDEG™ technology have been described for the treatment of antibody-mediated diseases such as autoimmune diseases (see WO2006/130834 and WO2015/100299, incorporated herein by reference).

Additional Fc domain alterations that may be incorporated into the variant Fc domains or FcRn binding fragments also include without limitation those disclosed in Ghetie et al., 1997, Nat. Biotech. 15:637-40; Duncan et al, 1988, Nature 332:563-564; Lund et al., 1991, J. Immunol., 147:2657-2662; Lund et al, 1992, Mol. Immunol., 29:53-59; Alegre et al, 1994, Transplantation 57:1537-1543; Hutchins et al., 1995, Proc Natl. Acad Sci USA, 92:11980-11984; Jefferis et al, 1995, Immunol Lett., 44:111-117; Lund et al., 1995, Faseb J., 9:115-119; Jefferis et al, 1996, Immunol Lett., 54:101-104; Lund et al, 1996, J. Immunol., 157:4963-4969; Armour et al., 1999, Eur J Immunol. 29:2613-2624; Idusogie et al, 2000, J. Immunol., 164:4178-4184; Reddy et al, 2000, J. Immunol., 164:1925-1933; Xu et al., 2000, Cell Immunol., 200:16-26; Idusogie et al, 2001, J. Immunol., 166:2571-2575; Shields et al., 2001, J Biol. Chem., 276:6591-6604; Jefferis et al, 2002, Immunol Lett., 82:57-65; Presta et al., 2002, Biochem Soc Trans., 30:487-490); U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; WO 04/063351, the contents of which are incorporated by reference herein in their entirety.

In certain embodiments, the antibodies described herein comprise a modified human IgG Fc domain consisting of up to 2, up to 3, up to 4, up to 5, up to 6, up to 7, up to 8, up to 9, up to 10, up to 12, up to 15, up to 20 substitutions relative to the corresponding wild-type IgG sequence.

Any of the galectin-10 antibodies described herein may exhibit pH-dependent antigen binding i.e. pH-dependent binding to galectin-10.

Antibodies that have bound antigen are taken up into cells and trafficked to the endosomal-lysosomal degradation pathway. Antibodies that are able to dissociate from their antigen in the early endosome can be recycled back to the cell surface. Antibodies that bind with high affinity to their antigen in the endosomal compartments are typically trafficked to the lysosomes for degradation. It has been shown previously that if an antibody has pH-dependent antigen binding activity, such that it has a lower binding affinity for its antigen at early endosomal pH as compared with plasma pH, the antibody will recycle to the cell surface more efficiently. This can extend the antibody plasma half-life and allow the same antibody to bind to multiple antigens. For this reason, it is advantageous for the anti-galectin-10 antibodies described herein to exhibit pH-dependent antigen binding. pH-dependent anti-galectin-10 antibodies in accordance with the present invention have the potential to eliminate galectin-10 by binding to this protein. The galectin-10 may then be released in the acidic endosomal compartment and trafficked to the lysosomes for degradation. The free anti-galectin-10 antibodies of the invention may then be recycled to the cell surface such that they can bind and internalise further galectin-10.

The anti-galectin-10 antibodies of the invention may possess intrinsic pH-dependent antigen binding activity i.e. they may have been selected for this property. Alternatively or in addition, the anti-galectin-10 antibodies described herein may be engineered so as to exhibit pH-dependent target binding. Methods of engineering pH-dependent antigen binding activity in antibody molecules are described in, for example, EP2275443, which is incorporated herein by reference. Methods of engineering pH-dependent antigen binding in antibody molecules are also described in WO2018/206748, which is incorporated herein by reference. The antibodies described herein may be modified by any technique so as to achieve pH-dependent binding. For example, the antibodies may be modified in accordance with the methods described in EP2275443 or WO2018/206748 such that they exhibit pH-dependent antigen binding.

For pH-dependent embodiments of the anti-galectin-10 antibodies described herein, the antigen-binding activity is lower at endosomal pH as compared to the antigen-binding activity at plasma pH. The endosomal pH is typically acidic pH whereas the plasma pH is typically neutral pH. Accordingly, the antibodies described herein, may exhibit pH-dependent antigen binding such that their antigen-binding activity is lower at acidic pH as compared to the antigen-binding activity at neutral pH. Endosomal pH or “acidic pH” may be pH of from about pH 4.0 to about pH 6.5, preferably from about pH 5.5 to about pH 6.5, preferably from about pH 5.5 to about pH 6.0, preferably pH 5.5, pH 5.6, pH 5.7 or pH 5.8. Plasma pH or “neutral pH” may be pH of from about pH 6.9 to about pH 8.0, preferably from about pH 7.0 to about pH 8.0, preferably from about pH 7.0 to about pH 7.4, preferably pH 7.0 or pH 7.4.

In certain embodiments, the anti-galectin-10 antibodies exhibit pH-dependent binding such that the antigen-binding activity at pH 5.8 is lower as compared with the antigen-binding activity at pH 7.4. The pH-dependent anti-galectin-10 antibodies may be characterised in that the dissociation constant (KD) for the antibody-antigen interaction at acidic pH or pH 5.8 is higher than the dissociation constant (KD) for the antibody-antigen interaction at neutral pH or at pH 7.4. In certain embodiments, the anti-galectin-10 antibodies exhibit pH-dependent binding such that the ratio of KD for the antigen at pH 5.8 and KD for the antigen at pH 7.4 (KD (pH5.8)/KD (pH7.4) is 2 or more, 4 or more, 6 or more, 8 or more, 10 or more, 12 or more.

The pH-dependent antigen-binding activity of an antibody molecule may be engineered by modifying an antibody molecule so as to impair the antigen-binding ability at acidic pH and/or increase the antigen-binding ability at neutral pH. For example, the antibody molecule may be modified by substituting at least one amino acid of the antibody molecule with histidine, or by inserting at least one histidine into the antibody molecule. Such histidine mutation (substitution or insertion) sites are not particularly limited, and any site is acceptable as long as the antigen-binding activity at endosomal pH (for example pH 5.8) is lower than that at plasma pH (for example pH 7.4) as compared to before the mutation or insertion.

In certain embodiments, the anti-galectin-10 antibodies may be engineered so as to exhibit pH-dependent antigen binding by the introduction of one or more substitutions into the variable domains. In preferred embodiments, the anti-galectin-10 antibodies are engineered so as to exhibit pH-dependent antigen binding by introducing one or more substitutions into one or more CDRs of the antibody. The substitutions may introduce one or more His residues into one or more sites of the variable domains, preferably the heavy chain and/or light chain CDRs so as to confer pH-dependent antigen binding.

For embodiments of the invention wherein the antibody comprises three heavy chain CDR sequences and three light chain CDR sequences, the six CDRs combined may consist of a total of 1-10 His substitutions, optionally 1-5 His substitutions, optionally 1, 2, 3, 4, 5, 6, 7, 8, 9 or 10 His substitutions. The anti-galectin-10 antibodies may be engineered in accordance with the methods described in WO2018/206748, incorporated herein by reference. Non-histidine substitutions may also be incorporated into variable domains, particularly the CDRs, of the pH-dependent antibodies described herein.

In preferred embodiments, the exemplary anti-galectin-10 antibodies having the particular CDR, VH and/or VL domain sequences recited herein are engineered such that they exhibit pH-dependent antigen binding. For example, the CDR sequences of the exemplary anti-galectin-10 antibodies described herein may be modified by the introduction of one or more Histidine substitutions so as to produce antibodies exhibiting pH-dependent antigen binding.

The antibodies described herein may also be modified so as to form immunoconjugates comprising an antibody conjugated to a cytotoxic agent such as a chemotherapeutic agent, toxin (i.e., an enzymatically active toxin of bacterial, fungal, plant or animal origin, or fragments thereof), or a radioactive isotope (i.e., a radioconjugate). Fc regions may also be engineered for half-life extension, as described by Chan and Carter (2010) Nature Reviews: Immunology 10:301-316, incorporated herein by reference.

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids.

In particular embodiments, the Fc region may be engineered such that there is no effector function. In certain embodiments, the antibody molecules of the invention may have an Fc region derived from naturally-occurring IgG isotypes having reduced effector function, for example IgG4. Fc regions derived from IgG4 may be further modified to increase therapeutic utility, for example by the introduction of modifications that minimise the exchange of arms between IgG4 molecules in vivo. Fc regions derived from IgG4 may be modified to include the S228P substitution.

In certain embodiments, the antibody molecules are modified with respect to glycosylation. For example, an aglycoslated antibody can be made (i.e., the antibody lacks glycosylation). Glycosylation can be altered to, for example, increase the affinity of the antibody for the target antigen. Such carbohydrate modifications can be accomplished by; for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen.

Also envisaged are variant antibodies that bind galectin-10 having an altered type of glycosylation, such as a hypofucosylated antibody having reduced amounts of fucosyl residues or a fully or partially de-fucosylated antibody (as described by Natsume et al., Drug Design Development and Therapy, Vol. 3, pp 7-16, 2009) or an antibody having increased bisecting GlcNac structures. Such altered glycosylation patterns have been demonstrated to increase the ADCC activity of antibodies, producing typically 10-fold enhancement of ADCC relative to an equivalent antibody comprising a “native” human Fc domain. Such carbohydrate modifications can be accomplished by, for example, expressing the antibody in a host cell with altered glycosylation enzymatic machinery (as described by Yamane-Ohnuki and Satoh, mAbs 1:3, 230-236, 2009). Examples of non-fucosylated antibodies with enhanced ADCC function are those produced using the Potelligent™ technology of BioWa Inc.

### C. Exemplary Antibodies that Bind Galectin-10

The present invention provides exemplary antibodies and antigen binding fragments that bind galectin-10. The antibodies and antigen binding fragments of the invention may be defined exclusively with respect to their structural characteristics, as described below.

[clone g24F02_N53A]

Provided herein is an antibody or antigen binding fragment that binds to galectin-10, wherein the antibody or antigen binding fragment comprises a variable heavy chain (VH) domain and a variable light chain (VL) domain wherein:
(i) the VH domain comprises the CDR sequences of HCDR3 comprising or consisting of SEQ ID NO: 2; HCDR2 comprising or consisting of SEQ ID NO: 3; HCDR1 comprising or consisting of SEQ ID NO: 1; and
(ii) the VL domain comprises the CDR sequences of LCDR3 comprising or consisting of SEQ ID NO: 8; LCDR2 comprising or consisting of SEQ ID NO: 9; LCDR1 comprising or consisting of SEQ ID NO: 7.

Also provided herein is an antibody or antigen binding fragment that binds to galectin-10, wherein the antibody or antigen binding fragment comprises a variable heavy chain (VH) domain and a variable light chain (VL) domain wherein:
(i) the VH domain comprises the amino acid sequence of SEQ ID NO: 4 or an amino acid sequence at least 90%, 95%, 97%, 98% or 99% identical thereto; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 90% identity to SEQ ID NO: 4; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 95% identity to SEQ ID NO: 4; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 97% identity to SEQ ID NO: 4; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 98% identity to SEQ ID NO: 4; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 99% identity to SEQ ID NO: 4; and
(ii) the VL domain comprises the amino acid sequence of SEQ ID NO: 10 or an amino acid sequence at least 90%, 95%, 97%, 98% or 99% identical thereto.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 4 or an amino acid sequence at least 90%, 95%, 97%, 98% or 99% identical thereto; and
(ii) the VL domain comprises the amino acid sequence with at least 90% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 4 or an amino acid sequence at least 90%, 95%, 97%, 98% or 99% identical thereto; and
(ii) the VL domain comprises the amino acid sequence with at least 95% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 4 or an amino acid sequence at least 90%, 95%, 97%, 98% or 99% identical thereto; and
(ii) the VL domain comprises the amino acid sequence with at least 97% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 4 or an amino acid sequence at least 90%, 95%, 97%, 98% or 99% identical thereto; and
(ii) the VL domain comprises the amino acid sequence with at least 98% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 4 or an amino acid sequence at least 90%, 95%, 97%, 98% or 99% identical thereto; and
(ii) the VL domain comprises the amino acid sequence with at least 99% identity to SEQ ID NO: 10.

In some embodiments, the antibody or antigen binding fragment comprises a variable heavy chain (VH) domain and a variable light chain (VL) domain wherein;
(i) the VH domain comprises the amino acid sequence of SEQ ID NO: 4; and
(ii) the VL domain comprises the amino acid sequence of SEQ ID NO: 10.

In some embodiments, the antibody or antigen binding fragment comprises a variable heavy chain (VH) domain and a variable light chain (VL) domain wherein;
(i) the VH domain consists of the amino acid sequence of SEQ ID NO: 4; and
(ii) the VL domain consists of the amino acid sequence of SEQ ID NO: 10.
   [clone g24F02]

Further provided herein is an antibody or antigen binding fragment that binds to galectin-10, wherein the antibody or antigen binding fragment comprises a variable heavy chain (VH) domain and a variable light chain (VL) domain wherein:
(i) the VH domain comprises the CDR sequences of HCDR3 comprising or consisting of SEQ ID NO: 2; HCDR2 comprising or consisting of SEQ ID NO: 5; HCDR1 comprising or consisting of SEQ ID NO: 1; and
(ii) the VL domain comprises the CDR sequences of LCDR3 comprising or consisting of SEQ ID NO: 8; LCDR2 comprising or consisting of SEQ ID NO: 9; LCDR1 comprising or consisting of SEQ ID NO: 7.

Also provided herein is an antibody or antigen binding fragment that binds to galectin-10, wherein the antibody or antigen binding fragment comprises a variable heavy chain (VH) domain and a variable light chain (VL) domain wherein:
(i) the VH domain comprises the amino acid sequence of SEQ ID NO: 6 or an amino acid sequence at least 90%, 95%, 97%, 98% or 99% identical thereto; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 90% identity to SEQ ID NO: 6; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 95% identity to SEQ ID NO: 6; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 97% identity to SEQ ID NO: 6; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 98% identity to SEQ ID NO: 6; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 99% identity to SEQ ID NO: 6; and
(ii) the VL domain comprises the amino acid sequence of SEQ ID NO: 10 or an amino acid sequence at least 90%, 95%, 97%, 98% or 99% identical thereto.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 6 or an amino acid sequence at least 90%, 95%, 97%, 98% or 99% identical thereto; and
(ii) the VL domain comprises the amino acid sequence with at least 90% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 6 or an amino acid sequence at least 90%, 95%, 97%, 98% or 99% identical thereto; and
(ii) the VL domain comprises the amino acid sequence with at least 95% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 6 or an amino acid sequence at least 90%, 95%, 97%, 98% or 99% identical thereto; and
(ii) the VL domain comprises the amino acid sequence with at least 97% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 6 or an amino acid sequence at least 90%, 95%, 97%, 98% or 99% identical thereto; and
(ii) the VL domain comprises the amino acid sequence with at least 98% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 6 or an amino acid sequence at least 90%, 95%, 97%, 98% or 99% identical thereto; and
(ii) the VL domain comprises the amino acid sequence with at least 99% identity to SEQ ID NO: 10.

In some embodiments, the antibody or antigen binding fragment comprises a variable heavy chain (VH) domain and a variable light chain (VL) domain wherein;
(i) the VH domain comprises the amino acid sequence of SEQ ID NO: 6; and
(ii) the VL domain comprises the amino acid sequence of SEQ ID NO: 10.

In some embodiments, the antibody or antigen binding fragment comprises a variable heavy chain (VH) domain and a variable light chain (VL) domain wherein;
(i) the VH domain consists of the amino acid sequence of SEQ ID NO: 6; and
(ii) the VL domain consists of the amino acid sequence of SEQ ID NO: 10.
   [clone g23H09]

Further provided herein is an antibody or antigen binding fragment that binds to galectin-10, wherein the antibody or antigen binding fragment comprises a variable heavy chain (VH) domain and a variable light chain (VL) domain wherein:
(i) the VH domain comprises the CDR sequences of HCDR3 comprising or consisting of SEQ ID NO: 12; HCDR2 comprising or consisting of SEQ ID NO: 13; HCDR1 comprising or consisting of SEQ ID NO: 11; and
(ii) the VL domain comprises the CDR sequences of LCDR3 comprising or consisting of SEQ ID NO: 8; LCDR2 comprising or consisting of SEQ ID NO: 9; LCDR1 comprising or consisting of SEQ ID NO: 7.

Also provided herein is an antibody or antigen binding fragment that binds to galectin-10, wherein the antibody or antigen binding fragment comprises a variable heavy chain (VH) domain and a variable light chain (VL) domain wherein:
(i) the VH domain comprises the amino acid sequence of SEQ ID NO: 14 or an amino acid sequence at least 90%, 95%, 97%, 98% or 99% identical thereto; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 90% identity to SEQ ID NO: 14; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 95% identity to SEQ ID NO: 14; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 97% identity to SEQ ID NO: 14; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 98% identity to SEQ ID NO: 14; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 99% identity to SEQ ID NO: 14; and
(ii) the VL domain comprises the amino acid sequence of SEQ ID NO: 10 or an amino acid sequence at least 90%, 95%, 97%, 98% or 99% identical thereto.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 14 or an amino acid sequence at least 90%, 95%, 97%, 98% or 99% identical thereto; and
(ii) the VL domain comprises the amino acid sequence with at least 90% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 14 or an amino acid sequence at least 90%, 95%, 97%, 98% or 99% identical thereto; and
(ii) the VL domain comprises the amino acid sequence with at least 95% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 14 or an amino acid sequence at least 90%, 95%, 97%, 98% or 99% identical thereto; and
(ii) the VL domain comprises the amino acid sequence with at least 97% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 14 or an amino acid sequence at least 90%, 95%, 97%, 98% or 99% identical thereto; and
(ii) the VL domain comprises the amino acid sequence with at least 98% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 14 or an amino acid sequence at least 90%, 95%, 97%, 98% or 99% identical thereto; and
(ii) the VL domain comprises the amino acid sequence with at least 99% identity to SEQ ID NO: 10.

In some embodiments, the antibody or antigen binding fragment comprises a variable heavy chain (VH) domain and a variable light chain (VL) domain wherein;
(i) the VH domain comprises the amino acid sequence of SEQ ID NO: 14; and
(ii) the VL domain comprises the amino acid sequence of SEQ ID NO: 10.

In some embodiments, the antibody or antigen binding fragment comprises a variable heavy chain (VH) domain and a variable light chain (VL) domain wherein;
(i) the VH domain consists of the amino acid sequence of SEQ ID NO: 14; and
(ii) the VL domain consists of the amino acid sequence of SEQ ID NO: 10.
   [clone g18C06]

Further provided herein is an antibody or antigen binding fragment that binds to galectin-10, wherein the antibody or antigen binding fragment comprises a variable heavy chain (VH) domain and a variable light chain (VL) domain wherein:
(i) the VH domain comprises the CDR sequences of HCDR3 comprising or consisting of SEQ ID NO: 16; HCDR2 comprising or consisting of SEQ ID NO: 17; HCDR1 comprising or consisting of SEQ ID NO: 15; and
(ii) the VL domain comprises the CDR sequences of LCDR3 comprising or consisting of SEQ ID NO: 20; LCDR2 comprising or consisting of SEQ ID NO: 21; LCDR1 comprising or consisting of SEQ ID NO: 19.

Also provided herein is an antibody or antigen binding fragment that binds to galectin-10, wherein the antibody or antigen binding fragment comprises a variable heavy chain (VH) domain and a variable light chain (VL) domain wherein:
(i) the VH domain comprises the amino acid sequence of SEQ ID NO: 18 or an amino acid sequence at least 90%, 95%, 97%, 98% or 99% identical thereto; and
(ii) the VL domain comprises the amino acid sequence of SEQ ID NO: 22 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 90% identity to SEQ ID NO: 18; and
(ii) the VL domain comprises the amino acid sequence of SEQ ID NO: 22 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 95% identity to SEQ ID NO: 18; and
(ii) the VL domain comprises the amino acid sequence of SEQ ID NO: 22 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 97% identity to SEQ ID NO: 18; and
(ii) the VL domain comprises the amino acid sequence of SEQ ID NO: 22 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 98% identity to SEQ ID NO: 18; and
(ii) the VL domain comprises the amino acid sequence of SEQ ID NO: 22 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 99% identity to SEQ ID NO: 18; and
(ii) the VL domain comprises the amino acid sequence of SEQ ID NO: 22 or an amino acid sequence at least 90%, 95%, 97%, 98% or 99% identical thereto.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 18 or an amino acid sequence at least 90%, 95%, 97%, 98% or 99% identical thereto; and
(ii) the VL domain comprises the amino acid sequence with at least 90% identity to SEQ ID NO: 22.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 18 or an amino acid sequence at least 90%, 95%, 97%, 98% or 99% identical thereto; and
(ii) the VL domain comprises the amino acid sequence with at least 95% identity to SEQ ID NO: 22.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 18 or an amino acid sequence at least 90%, 95%, 97%, 98% or 99% identical thereto; and
(ii) the VL domain comprises the amino acid sequence with at least 97% identity to SEQ ID NO: 22.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 18 or an amino acid sequence at least 90%, 95%, 97%, 98% or 99% identical thereto; and
(ii) the VL domain comprises the amino acid sequence with at least 98% identity to SEQ ID NO: 22.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 18 or an amino acid sequence at least 90%, 95%, 97%, 98% or 99% identical thereto; and
(ii) the VL domain comprises the amino acid sequence with at least 99% identity to SEQ ID NO: 22.

In some embodiments, the antibody or antigen binding fragment comprises a variable heavy chain (VH) domain and a variable light chain (VL) domain wherein;
(i) the VH domain comprises the amino acid sequence of SEQ ID NO: 18; and
(ii) the VL domain comprises the amino acid sequence of SEQ ID NO: 22.

In some embodiments, the antibody or antigen binding fragment comprises a variable heavy chain (VH) domain and a variable light chain (VL) domain wherein;
(i) the VH domain consists of the amino acid sequence of SEQ ID NO: 18; and
(ii) the VL domain consists of the amino acid sequence of SEQ ID NO: 22.
   [clone g20H09]

Further provided herein is an antibody or antigen binding fragment that binds to galectin-10, wherein the antibody or antigen binding fragment comprises a variable heavy chain (VH) domain and a variable light chain (VL) domain wherein:
(i) the VH domain comprises the CDR sequences of HCDR3 comprising or consisting of SEQ ID NO: 12; HCDR2 comprising or consisting of SEQ ID NO: 23; HCDR1 comprising or consisting of SEQ ID NO: 11; and
(ii) the VL domain comprises the CDR sequences of LCDR3 comprising or consisting of SEQ ID NO: 8; LCDR2 comprising or consisting of SEQ ID NO: 9; LCDR1 comprising or consisting of SEQ ID NO: 7.

Also provided herein is an antibody or antigen binding fragment that binds to galectin-10, wherein the antibody or antigen binding fragment comprises a variable heavy chain (VH) domain and a variable light chain (VL) domain wherein:
(i) the VH domain comprises the amino acid sequence of SEQ ID NO: 24 or an amino acid sequence at least 90%, 95%, 97%, 98% or 99% identical thereto; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 90% identity to SEQ ID NO: 24; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 95% identity to SEQ ID NO: 24; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 97% identity to SEQ ID NO: 24; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 98% identity to SEQ ID NO: 24; and
(ii) the VL domain comprises the amino acid sequence of SEQ ID NO: 10 or an amino acid sequence at least 90%, 95%, 97%, 98% or 99% identical thereto.

In some embodiments, the antibody or antigen binding fragment that bind to galectin-10, comprises a variable heavy chain (VH) domain and a variable light chain (VL) domain wherein:
(i) the VH domain comprises an amino acid sequence with at least 99% identity to SEQ ID NO: 24; and
(ii) the VL domain comprises the amino acid sequence of SEQ ID NO: 10 or an amino acid sequence at least 90%, 95%, 97%, 98% or 99% identical thereto.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 24 or an amino acid sequence at least 90%, 95%, 97%, 98% or 99% identical thereto; and
(ii) the VL domain comprises the amino acid sequence with at least 90% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 24 or an amino acid sequence at least 90%, 95%, 97%, 98% or 99% identical thereto; and
(ii) the VL domain comprises the amino acid sequence with at least 95% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 24 or an amino acid sequence at least 90%, 95%, 97%, 98% or 99% identical thereto; and
(ii) the VL domain comprises the amino acid sequence with at least 97% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 24 or an amino acid sequence at least 90%, 95%, 97%, 98% or 99% identical thereto; and
(ii) the VL domain comprises the amino acid sequence with at least 98% identity to SEQ ID NO: 10.

In some embodiment, the antibody or antigen binding fragment that bind to galectin-10, comprises a variable heavy chain (VH) domain and a variable light chain (VL) domain wherein:
(i) the VH domain comprises the amino acid sequence of SEQ ID NO: 24 or an amino acid sequence at least 90%, 95%, 97%, 98% or 99% identical thereto; and
(ii) the VL domain comprises the amino acid sequence with at least 99% identity to SEQ ID NO: 10.

In some embodiments, the antibody or antigen binding fragment comprises a variable heavy chain (VH) domain and a variable light chain (VL) domain wherein;
(i) the VH domain comprises the amino acid sequence of SEQ ID NO: 24; and
(ii) the VL domain comprises the amino acid sequence of SEQ ID NO: 10.

In some embodiments, the antibody or antigen binding fragment comprises a variable heavy chain (VH) domain and a variable light chain (VL) domain wherein;
(i) the VH domain consists of the amino acid sequence of SEQ ID NO: 24; and (ii) the VL domain consists of the amino acid sequence of SEQ ID NO: 10.

For embodiments wherein the domains of the antibodies or antigen binding fragments are defined by a particular percentage sequence identity to a reference sequence, the VH and/or VL domains may retain identical CDR sequences to those present in the reference sequence such that the variation is present only within the framework regions. In an alternative embodiment, the CDR sequences may also comprise amino acid substitutions (e.g., conservative substitutions, humanising substitutions or affinity variants) relative to the reference sequence.

The invention also provides antibodies or antigen binding fragments thereof, which bind to the same epitope as the galectin-10 antibodies exemplified herein.

In certain embodiments, the exemplary antibodies and antigen binding fragments defined as having the CDR sequences recited above or defined as having a particular percentage identity to the specific VH/VL domain amino acid sequences recited above are humanised, germlined or affinity variants of the antibodies or antigen binding fragments thereof from which the CDR, VH and/or VL sequences derive.

In a preferred embodiment, the exemplary antibody molecules having the CDR sequences recited above exhibit high human homology, for example are humanised or germlined variants of the antibodies or antigen binding fragments thereof from which the CDR sequences derive.

For antibody molecules intended for human therapeutic use, it is typical for the entire constant region of the antibody, or at least a part thereof, to have fully or substantially human amino acid sequence. Therefore, in one embodiment, the Fc region may be fully or substantially human with respect to its amino acid sequence. In the context of the constant region of a humanised or chimeric antibody, or an antibody fragment, the term “substantially human” refers to an amino acid sequence identity of at least 90%, or at least 92%, or at least 95%, or at least 97%, or at least 99% with a human constant region. The term “human amino acid sequence” in this context refers to an amino acid sequence which is encoded by a human immunoglobulin gene, which includes germline, rearranged and somatically mutated genes. The invention also contemplates polypeptides comprising constant domains of “human” sequence which have been altered, by one or more amino acid additions, deletions or substitutions with respect to the human sequence, excepting those embodiments where the presence of a “fully human” hinge region is expressly required. Any of the exemplary Fc region modifications described herein may be incorporated into the antibodies having the CDR and/or VH/VL domain sequences recited above. In certain embodiments, the antibodies having the CDR and/or VH/VL domain sequences recited above comprise a modified human IgG Fc domain comprising or consisting of the amino acid substitutions H433K and N434F, wherein the Fc domain numbering is in accordance with EU numbering. In certain embodiments, the antibodies having the CDR and/or VH/VL domain sequences recited above comprise a modified human IgG Fc domain comprising or consisting of the amino acid substitutions M252Y, S254T, T256E, H433K and N434F.

### D. Polynucleotides Encoding Antibodies that Bind to Galectin-10

The invention also provides polynucleotide molecules encoding the galectin-10 antibodies of the invention or fragments thereof. Polynucleotide molecules encoding the full-length galectin-10 antibodies are provided, together with polynucleotide molecules encoding fragments, for example the VH and/or VL domains of the galectin-10 antibodies described herein. Also provided are expression vectors containing said nucleotide sequences of the invention operably linked to regulatory sequences which permit expression of the antibodies or fragments thereof in a host cell or cell-free expression system, and a host cell or cell-free expression system containing this expression vector.

Polynucleotide molecules encoding galectin-10 antibodies of the invention include, for example, recombinant DNA molecules. The terms “nucleic acid”, “polynucleotide” or a “polynucleotide molecule” as used herein interchangeably and refer to any DNA or RNA molecule, either single- or double-stranded and, if single-stranded, the molecule of its complementary sequence. In discussing nucleic acid molecules, a sequence or structure of a particular nucleic acid molecule may be described herein according to the normal convention of providing the sequence in the 5′ to 3′ direction. In some embodiments of the invention, nucleic acids or polynucleotides are “isolated.” This term, when applied to a nucleic acid molecule, refers to a nucleic acid molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or non-human host organism. When applied to RNA, the term “isolated polynucleotide” refers primarily to an RNA molecule encoded by an isolated DNA molecule as defined above. Alternatively, the term may refer to an RNA molecule that has been purified/separated from other nucleic acids with which it would be associated in its natural state (i.e., in cells or tissues). An isolated polynucleotide (either DNA or RNA) may further represent a molecule produced directly by biological or synthetic means and separated from other components present during its production.

For recombinant production of a galectin-10 antibody according to the invention, a recombinant polynucleotide encoding it or recombinant polynucleotides encoding the different chains or domains may be prepared (using standard molecular biology techniques) and inserted into a replicable vector for expression in a chosen host cell, or a cell-free expression system. Suitable host cells may be prokaryote, yeast, or higher eukaryote cells, specifically mammalian cells. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen. Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); mouse myeloma cells SP2/0-AG14 (ATCC CRL 1581; ATCC CRL 8287) or NS0 (HPA culture collections no. 85110503); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), as well as DSM's PERC-6 cell line. Expression vectors suitable for use in each of these host cells are also generally known in the art.

It should be noted that the term “host cell” generally refers to a cultured cell line. Whole human beings into which an expression vector encoding an antigen binding polypeptide according to the invention has been introduced are explicitly excluded from the definition of a “host cell”.

### E. Antibody Production

In a further aspect, the invention also provides a method of producing antibodies of the invention which comprises culturing a host cell (or cell free expression system) containing polynucleotide (e.g. an expression vector) encoding the antibody under conditions which permit expression of the antibody, and recovering the expressed antibody. This recombinant expression process can be used for large scale production of antibodies, including galectin-10 antibodies according to the invention, including monoclonal antibodies intended for human therapeutic use. Suitable vectors, cell lines and production processes for large scale manufacture of recombinant antibodies suitable for in vivo therapeutic use are generally available in the art and will be well known to the skilled person.

### F. Pharmaceutical Compositions

The invention includes pharmaceutical compositions, containing one or a combination of galectin-10 antibodies or antigen binding fragments thereof, formulated with one or more pharmaceutically acceptable carriers or excipients. Such compositions may include one or a combination of (i.e., two or more different) galectin-10 antibodies. Techniques for formulating monoclonal antibodies for human therapeutic use are well known in the art and are reviewed, for example, in Wang et al., Journal of Pharmaceutical Sciences, Vol. 96, pp 1-26, 2007, the contents of which are incorporated herein in their entirety.

The pharmaceutical composition according to the invention may be administered alone or in combination with other treatments, either simultaneously or sequentially.

Pharmaceutically acceptable excipients that may be used to formulate the compositions include, but are not limited to: ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In certain embodiments, the compositions are formulated for administration to a subject via any suitable route of administration including but not limited to intramuscular, intravenous, intradermal, intraperitoneal injection, subcutaneous, epidural, nasal, oral, rectal, topical, inhalational, buccal (e.g., sublingual), and transdermal administration.

In a preferred embodiment, the route of administration is inhalational. Suitably, the compositions of the invention can be formulated as a powder for inhalation or as an aerosolised liquid for inhalation. Suitably, the compositions according to the invention may be formulated as a dry powder. Alternatively, the compositions according to the invention may be formulated as a nebulized liquid aerosol or a liquid spray.

Means and devices for inhaled administration of compositions are well known in the art. Inhalational administration of a composition can, for example, be achieved via a nebulizer. A nebulizer is a drug delivery device that is used to administer medication as a mist that is inhaled into the lungs. In the alternative, an inhaler can be used to administer the compositions of the invention. An inhaler is a drug delivery device that delivers medications into the lungs vie inhalation. Several types of inhalers are well-known in the art, including for example metered-dose inhalers (MDIs), dry powder inhalers (DPIs) and soft mist inhalers (SMIs).

### G. Methods of Treatment

The antibodies and antigen binding fragments that bind to galectin-10 that are described herein, may be used in methods of treatment. Thus, the invention provides an antibody and antigen binding fragment that binds to galectin-10 for use as a medicament. Alternatively, provided herein is an antibody and antigen binding fragment that binds to galectin-10 for use in a method of treatment. The antibodies and antigen binding fragments of the invention that are for use as medicaments are typically formulated as pharmaceutical compositions.

Importantly, all embodiments described above in relation to the antibodies and antigen binding fragments, are equally applicable to the methods described herein.

The present invention also provides methods of treating a subject in need thereof, wherein the method comprises administering to the subject a therapeutically effective amount of an antibody or antigen binding fragment as described elsewhere herein. In such methods of treatment, the antibodies and antigen binding fragments are typically formulated as pharmaceutical compositions. As used herein, the term “therapeutically effective amount” is intended to mean the quantity or dose of galectin-10 antibody, that is sufficient to produce a therapeutic effect, for example, the quantity or dose of antagonist required to eradicate or at least alleviate the symptoms associated with a disease or condition. An appropriate amount or dose can be determined by a physician, as appropriate. For example, the dose can be adjusted based on factors such as the size or weight of a subject to be treated, the age of the subject to be treated, the general physical condition of the subject to be treated, the condition to be treated, and the route of administration.

For clinical use, in certain embodiments, the galectin-10 antibody or antigen binding fragment as described elsewhere herein is administered to a subject as one or more doses of about 0.1 mg/kg body weight to about 20 mg/kg body weight. In certain embodiments, the antibody or antigen binding fragment as described elsewhere herein is administered to a subject in a dose of about 0.1 mg/kg body weight to about 10 mg/kg body weight. In certain embodiments, the antibody or antigen binding fragment as described elsewhere herein is administered to a subject in a dose of about 0.5 mg/kg body weight to about 10 mg/kg body weight. In certain embodiments, the antibody or antigen binding fragment as described elsewhere herein is administered to a subject in a dose of about 1 mg/kg body weight to about 10 mg/kg body weight.

The antibodies and antigen binding fragments that bind galectin-10 are useful in therapeutic methods, for the reason that they can disrupt galectin-10 crystallization. As explained elsewhere herein, the antibodies of the present invention bind to an epitope of galectin-10 thereby disrupting the crystallization of galectin-10. In certain embodiments, the antibodies and antigen binding fragments inhibit the crystallization of galectin-10. In certain embodiments, the antibodies and antigen binding fragments promote dissolution of crystalline galectin-10.

The galectin-10 antibodies and antigen binding fragments thereof, may be for use in preventing or treating diseases or conditions associated with the presence or formation of galectin-10 crystals or CLCs. Provided herein are methods of preventing or treating a disease or condition associated with the presence or formation of galectin-10 crystals or CLCs in a patient or subject in need thereof by administering an effective amount of a galectin-10 antibody or antigen binding fragment thereof.

As used herein, a method of “preventing” a disease or condition means preventing the onset of the disease, preventing the worsening of symptoms, preventing the progression of the disease or condition or reducing the risk of a subject developing the disease or condition. As used herein, a method of “treating” a disease or condition means curing a disease or condition and/or alleviating or eradicating the symptoms associated with the disease or condition such that the patient's suffering is reduced.

For patients having diseases or conditions characterised by the presence of galectin-10 crystals, the methods of treatment will typically involve the administration of a galectin-10 antibody or antigen binding fragment thereof, capable of dissolving the galectin-10 crystals located in the patient's tissues. For patients identified as “at risk” of developing a disease or condition characterised by the formation of galectin-10 crystals, the methods of prevention may involve the administration of a galectin-10 antibody or antigen binding fragment thereof, capable of inhibiting the crystallization of galectin-10.

Galectin-10 crystals or CLCs have been observed in patients having a range of diseases and conditions. It follows that the galectin-10 antagonists described herein may be used to prevent or treat a disease or condition selected from the group consisting of: asthma; chronic rhinosinusitis; celiac disease; helminth infection; gastrointestinal eosinophilic inflammation; cystic fibrosis (CF); allergic bronchopulmonary aspergillosis (ABPA); Churg-Straus vasculitis; chronic eosinophilic pneumonia; and acute myeloid leukemia (AML). In preferred embodiments, galectin-10 antibodies or antigen binding fragments thereof are used to prevent or treat a disease or condition selected from the group consisting of: asthma; chronic rhinosinusitis; celiac disease; helminth infection; gastrointestinal eosinophilic inflammation; cystic fibrosis (CF); allergic bronchopulmonary aspergillosis (ABPA); Churg-Straus vasculitis; chronic eosinophilic pneumonia; and acute myeloid leukemia (AML).

As noted above, galectin-10 crystals or CLCs are particularly associated with diseases or conditions characterised by eosinophilic inflammation. In preferred embodiments therefore, the galectin-10 antibodies or antigen binding fragments thereof described herein, are used to treat disorders or conditions associated with eosinophilic inflammation.

In particularly preferred embodiments, the galectin-10 antibodies or antigen binding fragments thereof described herein are used to prevent or treat asthma. An analysis of the airways and lungs of asthmatic patients showed the presence of CLCs (Persson EK,

Verstraete K, Heyndrickx I, et al. Protein crystallization promotes type 2 immunity and is reversible by antibody treatment. Science. 2019; 364 (6442)). Therefore, the antibodies of the present invention bind to an epitope of galectin-10 and thereby disrupt the crystallization of galectin-10. This in turn prevents CLC formation in the airways and lungs of asthmatic patients.

Clinically, asthma is characterised by reversible airway obstruction and hyperresponsiveness leading to shortness of breath and wheezing. Although often treatable with inhaled steroids and bronchodilators, a subgroup of patients have severe therapy-resistant disease requiring frequent hospital admissions, which may lead to a fatal attack (Braido F. Failure in asthma control: reasons and consequences. Scientifica (Cairo) 2013; 2013:549252). Pathologically, the disease is characterized by airway eosinophilia and by excessive production of thickened mucus that can lead to irreversible obstruction of small airways (Zhang L, He L, Gong J, Liu C. Risk Factors Associated with Irreversible Airway Obstruction in Asthma: A Systematic Review and Meta-Analysis. Biomed Res Int. 2016; 2016:9868704). In most cases the disease is driven by type 2 immune cells (CD4 Th2 lymphocytes and type 2 innate lymphoid cells (ILC2)) immune response, leading to the production of IL-4 (stimulating goblet cell metaplasia and IgE synthesis), IL-5 (promoting tissue eosinophilia), and IL-13 (causing bronchial hyperreactivity and goblet cell metaplasia) (Lambrecht B N, Hammad H. The immunology of asthma. Nat Immunol. 2015; 16 (1): 45-56).

In some embodiments, the asthma is characterised as allergic asthma. Allergic asthma is a chronic inflammatory disease of the conducting airways affecting 8-12% of people in Europe (Selroos O, Kupczyk M, Kuna P, et al. National and regional asthma programmes in Europe. Eur Respir Rev. 2015; 24 (137): 474-483).

In other particularly preferred embodiments, the galectin-10 antibodies or antigen binding fragments thereof described herein, are used to prevent or treat cystic fibrosis (CF).

The present invention also provides use of the galectin-10 antibodies or antigen binding fragments thereof for the detection of galectin-10 in a sample obtained from a patient. The antibodies or antigen binding fragments thereof are typically used to detect crystalline galectin-10. As noted above, galectin-10 crystals or CLC crystals have been observed in patients having a number of different diseases and conditions. It follows, that the patient sample may be isolated from a subject having or suspected of having any one of the following diseases or conditions: asthma, chronic rhinosinusitis, celiac disease, helminth infection, gastrointestinal eosinophilic inflammation, cystic fibrosis (CF), allergic bronchopulmonary aspergillosis (ABPA), Churg-Straus vasculitis, chronic eosinophilic pneumonia, or acute myeloid leukemia (AML). The detection of crystalline galectin-10 in the patient sample may be used to diagnose the disease or condition in the subject from which the sample was obtained. The sample may be any suitable patient sample, for example any fluid or tissue in which CLCs are observed in a disease state. In certain embodiments, the sample is a tissue sample obtained from a polyp, for example a nasal polyp. In certain embodiments, the sample is a mucus sample. In such embodiments, the detection of crystalline galectin-10 in the mucus sample using the antibodies or antigen binding fragments thereof of the invention may be used to detect or diagnose chronic rhinosinusitis. In preferred embodiments, the patient sample is a sputum sample. In such embodiments, the detection of crystalline galectin-10 in the sputum sample using the antibodies or antigen binding fragments thereof of the invention may be used to detect or diagnose asthma.

### H. Kits

Any of the antibodies or antigen binding fragments described herein can be packaged as a kit and optionally include instructions for use.

### EXAMPLES

The invention will be further understood with reference to the following non-limiting examples.

### Background and Objectives

Clone 7B07 was described in WO 2019/197675. This clone was observed to bind and dissolve recombinant Charcot-Leyden crystals ((CLCs) also referred to as Galectin-10 (GAL10) crystals). The germlining process via complementarity determining region (CDR) grafting had no impact on the binding and potency of the clone. However, stability studies identified a deamidation site (N53G54) in the CDR2 of the heavy chain, which caused a decline in binding and potency at incubation temperatures of 25° C. and 37° C. In order to overcome this issue, variants of the germlined 7B07 (g7B07) clone were generated with point mutations at N53 and G54 in the CDR2 of the heavy chain. Whilst the potency of these g7B07 mutants to dissolve recombinant CLC was preserved, all mutations resulted in a drop in binding properties.

Consequently, three discovery campaigns were initiated to identify further anti-galectin-10 (anti-Gal10) compounds with more favorable properties as compared to the clone 7B07.

### Example 1. Selection of Recombinant Antibodies that Bind to Gal10

### 1.1 7B07 Epitope Campaign

### Selection of Clones Binding to the 7B07 Epitope on Gal10 Via Phage Display

To select the scFv clones with the appropriate binding capacity to human Gal10, a phage panning approach was used. To select clones binding to a similar region on Gal10 as clone 7B07 a competition set-up was used.

In this set-up, the anti-human specific clone 1D11, targeting the Tyrosine 69 residue was coated on a Maxisorp plate to capture Gal10-His. Capturing Gal10 with 1D11 had two advantages in selecting clones binding to the 7B07 epitope. The first advantage was that, because 1D11 binds to the opposite site on Gal10 to 7B07, the 7B07 epitope was accessible to the phages expressing scFv against Gal10. The second advantage was that by capturing Gal10-His with a clone binding to the Tyrosine 69 (clone 1D11) this epitope was masked. This is relevant because during the selection campaigns, it appeared that most of the clones bound close to the 1D11 epitope. Elution of the bound phages was done either with trypsin (non-specific elution) or via competitive elution with a high concentration of 7B07 IgG (specific elution of phage expressing scFv binding to a similar binding region to clone 7B07).

Two llama-derived scFv libraries (Lambda and Kappa) were used to select for scFv clones having binding activity for Gal10. Two rounds of selection resulted in a clear enrichment of phages expressing scFv specific for human Gal10. A similar enrichment (up to 100-fold) to the PBS control was observed.

### Screening for Gal10 Specific Binders

Two master plates were generated after round two of selections against the 7B07 epitope of Gal10 where both trypsin and competitive elution with 7B07 were used. Master plate 18 (MP18) was generated from the second round of selection of the Lambda library where competitive elution was performed from the first round. Master plate 19 (MP19) was generated from all the other conditions where elution was performed using trypsin or 7B07 (Table 3). From these master plates, periplasmic extracts were generated (scFv) and their binding capacity to Gal10 was analyzed by ELISA and Biacore.

TABLE 3
Overview of the master plates (MP) generated after the
selection campaign against the 7B07 epitope on Gal10.

| | | | Round | Round 1 | | |
|---|---|---|---|---|---|---|
| Plate | | | of | elution of | | |
| number | Llama | Library | selection | phages used | Elution | |
| MP18 | Ynigo | λ | 2 | Competitive | Competitive | YLC |
| | | | | | (7B07) | |
| MP19 | Montoyo | λ and κ | 2 | Trypsin | Trypsin | ML + MK |
| | Ynigo | λ and κ | 2 | Trypsin | Trypsin | YL + YK |
| | Montoyo | λ and κ | 2 | Competitive | Competitive | MLC + MKC |
| | | | | | (7B07) | |
| | Ynigo | κ | 2 | Competitive | Competitive | YKC |
| | | | | | (7B07) | |
| | Montoyo | λ and κ | 2 | Trypsin | Competitive | MLT + MKT |
| | | | | | (7B07) | |
| | Ynigo | λ and κ | 2 | Trypsin | Competitive | YLT + YKT |
| | | | | | (7B07) | |

Screening of the scFv Periplasmic Extracts

The binding capacity of the periplasmic extracts was analyzed by ELISA (binding and competition with 7B07) and Surface Plasmon Resonance (SPR).

### Binding Screening by ELISA

The binding capacity of the scFv (periplasmic extract) to human Gal10 was analyzed by ELISA. In this experiment, clones with an OD of 0.3 or higher were classified as Gal10 binders. In total, 48 Gal10-specific clones were identified.

### Competition Screening by ELISA

The target-binding region of the scFv on Gal10 was then analysed via an additional ELISA in which the competition of the clones against clone 7B07 was investigated. In this set-up, Gal10 was captured on a Maxisorp plate coated with 7B07. Therefore, it was expected that clones with a similar binding position to clone 7B07 on Gal10 would not be able to bind and would show low OD value, whereas clones binding to other regions would show a high OD value.

Analysis of the binding and competition ELISA experiments revealed that 25 clones competed with clone 7B07 for Gal10 binding. These clones showed an OD value >0.3 in the binding ELISA and <0.1 OD value in the competition ELISA.

### Off-Rate Screening on SPR (Biacore 3000)

The off-rate of the remaining 25 clones of the ELISA experiment was determined by SPR on a Biacore 3000 instrument. Periplasmic extracts were injected on a CM5 sensor chip coated with 2500 RU Gal10-His. Eleven clones showed at least a 2-fold improvement in off-rate as compared to clone 7B07 (2.18E-03 1/s) and these clones were selected for further characterization (Table 4).

TABLE 4

| | | Fold off-rate | |
|---|---|---|---|
| | Off-rate | compared | |
| Clone | (kd (1/s)) | to g7B07 | Rmax |
| 18C06 | 4.81E−04 | 0.22 | 1860 |
| 18H12 | 8.58E−04 | 0.39 | 222 |
| 18E04 | 9.61E−04 | 0.44 | 1170 |
| 18E05 | 9.63E−04 | 0.44 | 1570 |
| 18G07 | 9.63E−04 | 0.44 | 1690 |
| 18D09 | 1.19E−03 | 0.55 | 1010 |
| 19A04 | 2.27E−03 | 1.04 | 872 |
| 18G12 | 2.44E−03 | 1.12 | 938 |
| 19C10 | 3.32E−03 | 1.52 | 486 |
| 18G08 | 3.49E−03 | 1.60 | 243 |
| 18E01 | 4.37E−03 | 2.00 | 967 |
| 7B07 | 2.18E−-03 | 1.00 | 574 |
| (mean) | | | |

Off-rates of the scFv periplasmic extracts. This table indicates the amplitude of the binding (Rmax), the dissociation (off-rate) of each clone as well as the fold change in off-rate compared to the control (g7B07).

### 1.2 Heavy Chain Shuffling Campaign

A heavy chain shuffling approach was executed to find clones that pair with the 7B07 light chain and allow for good affinity to Gal10 and improved stability.

### Library Construction (VH-Shuffling of Fab)

For the construction of the shuffled heavy chain libraries, a two-step PCR was used. First, non-tagged primers were used directly on the cDNA of the two immunized llamas (obtained in the previous selection campaign) to amplify the VH-CH1. The obtained PCR product was then purified and used in a second PCR with tagged primers to amplify the VH. As clone 7B07 was isolated from llama Montoyo, the VL of clone 7B07 was shuffled with the PCR-amplified VH repertoire from llama Montoyo. The final Fab library size was 1.5E+07 VH/VL combinations, with a percentage of proper insertion of VL and VH at 94%, as determined by colony PCR.

To select new Fabs with a comparable or better binding capacity to human Gal10 than the parental g7B07 Fab, a phage panning approach was used. For this purpose, the first round and second round of selection were carried out on human Gal10-His and an unrelated His-tagged protein (as a control). A third and fourth round of selection were carried out on the soluble, non His-tagged human Gal10.

The first two rounds of selection were performed on 1 and 10 μg/mL of coated human Gal10-His and 10 μg/mL of the unrelated His-tagged protein. For both the first and second round of selection, the eluted phages from the condition 10 μg/mL Gal10-His were used for subsequent third and fourth rounds of selection.

### Screening for Gal10 Specific Binders

### Production of the Fabs as Periplasmic Extracts

From the trypsin-eluted phages of round 3 and round 4, single clones were generated and resulted in the creation of two master plates (Table 5). Master plate 24 (MP24) was created from the third round of selection with colonies selected from different conditions (Gal10, non-off-rate wash and off-rate wash). Master plate 26 (MP26) was created from the fourth round of selection with colonies selected from both non-off-rate wash and off-rate wash.

TABLE 5

| Plate | | | Round of | | Selected for Gal10 | | |
|---|---|---|---|---|---|---|---|
| number | Llama | Library | selection | Elution | at (μg/mL) | | |
| MP24 | Montoyo | λ | 3 | Trypsin | 25% | 5 | μg/ml |
| | | | | | | NORW | |
| | | | | | 25% | 0.5 | μg/mL |
| | | | | | | NORW | |
| | | | | | 50% | 5 | μg/mL |
| | | | | | | ON ORW | |
| MP26 | Montoyo | λ | 4 | Trypsin | 100% | 0.5 | μg/mL |

NORW

Overview of the master plates generated after the selection campaign against human Gal10.

### Sequence Analysis

Sequencing results of master plate 24 (MP24) revealed that, based on CDR3 sequences, only 4 groups of VH families distinct from 7B07_VH were present. Further analysis demonstrated that 2 of these 4 VH families were camelid single-domain antibodies. From the two remaining VH families, a representative clone was selected for further analysis-clone 24A04 and clone 24F02.

### Gal10 Binding of Two Selected Clones Using BLI Technology

Periplasmic extracts from the two selected clones were tested for binding to captured Gal10-His using the Octet RED96 instrument (Bio-Layer Interferometry (BLI) technology). In this analysis, a germlined clone of clone 7B07 (g7B07) was included as a reference. A low response to Gal10-His was measured compared to the reference clone. Only clone 24F02 showed a better (128-fold) off-rate compared to clone g7B07 (Table 6).

TABLE 6

| | | | kd fold |
|---|---|---|---|
| | Response | | difference |
| Clone | (nm) | kd (1/s) | to reference |
| g7B07 | 0.72 | 7.54E−03 | 1.0 |
| 24F02 | 0.19 | 5.86E−05 | 128.7 |
| 24A04 | 0.09 | 5.60E−02 | 0.1 |

Calculated off-rates kd (1/s).

### Competitive ELISA to the 7B07 Epitope

A competitive ELISA was performed to ensure that the selected clones target the same region on Gal10 as clone g7B07. Briefly, a 96-well Maxisorp plate was coated with 7B07_hIgG1 and Gal10-His was captured. Fab-Myc containing periplasmic extracts were then incubated, and bound Fab was detected with an anti-Myc-HRP antibody. Clones having an OD value <0.1 were defined as sharing the 7B07 epitope. A positive control sample (clone 18C06) was used as a reference sample.

Clone 24F02 did not show binding, suggesting that 24F02 binds to the same epitope as clone 7B07. Similar data were obtained for control antibody 18C06, which is known to compete with 7B07. Clone 24A04, on the contrary, showed an OD value >0.1 indicating that it binds to another epitope than 7B07.

TABLE 7

| | Average |
|---|---|
| Clone | OD value |
| 18C06 | 0.030 |
| 24A04 | 0.289 |
| 24F02 | 0.010 |

Average OD 450 nm values.

1.3 g7B07 CDR2 VH Randomization Campaign

### Library Construction (Fab)

Randomization of the deamidation site within g7B07 CDR2-N53G54-failed to provide a g7B07 variant without a deamidation site and good binding affinity to Gal10. CDR2 residues were randomized to find sequences without a deamidation site and good binding affinity. Structural modelling of part of the 7B07 Fab in complex with Gal10 directed the generation of randomized CDR2 libraries for further investigation. Only the flexible tip of the CDR2 loop (residues 52-57—KNGGGI) (SEQ ID NO: 74) was randomized and the antiparallel beta-sheet was left intact; four libraries were constructed. In the library labelled X6, all 6 residues of the flexible tip of the CDR2 loop were randomized (IXXXXXXT, where X represents one amino acid that was randomized). As this carries the risk of finding back the original 7B07 VH-CDR2 sequence, 3 additional libraries were constructed that were 1 amino acid shorter: X3 (IKXXXIT) (SEQ ID NO: 75); X4 (IXXXXT); and X5 (IXXXXXT); in which ‘X’ represents a randomized position.

In the crystal structure, residues 54-56 (GGG) collide with the Gal10 molecule and bend back G55. Therefore it was considered that making the sequence 1 amino acid shorter could be a better fit to bind Gal10.

To randomize the six residues in the CDR2 of the variable domain of the heavy chain of clone g7B07, a specific set of primers were generated for each library. After two steps of nested PCR using the DNA of the heavy chain of the germline clone 7B07 as a template, amplicons were digested with restriction enzymes before being ligated in the PCB13 phagemid vector containing the variable domain of the light chain of g7B07.

The library construction resulted in four libraries, which showed a 5-1224-fold higher library size than the theoretical library size (Table 8).

TABLE 8

| | X3 | X4 | X5 | X6 |
|---|---|---|---|---|
| Library size | 1.13E+07 | 4.50E+06 | 1.70E+07 | 7.05E+08 |
| (including | | | | |
| % insert) | | | | |
| Number of | 3 | 4 | 5 | 6 |
| randomized | residues | residues | residues | residues |
| residues | | | | |
| Theoretical | 9.3E+03 | 1.9E+05 | 4.1E+06 | 8.6E+07 |
| Library size | | | | |
| Fold | 1224 | 23 | 5 | 8 |
| compared | | | | |
| to Library | | | | |
| size | | | | |

Size of four different libraries that were generated.

### Selection Via Phage Display

To identify stable g7B07 variants with good binding affinity to Gal10, phages of the four different libraries (Input) were allowed to bind to Gal10-His coated on a Maxisorp plate in the presence or absence of off-rate washings with a 10-fold excess of Gal10-His over a 24 hour period. To assess specific enrichment against Gal10 and not the His-tag, an unrelated His-tagged protein was coated as negative control. This process resulted in clear enrichment after the first round of selection. The output titers of the unrelated His-tagged protein panning were clearly higher than on the PBS control. Additionally, the 24 hours off-rate wash performed with a 10-fold excess of Gal10 resulted in a lower enrichment (10-100-fold) compared to the elution performed at day 0, but similar to the elution control performed after 24 hours (data not shown), with most likely a phage expressing Fab with a higher affinity for human Gal10.

The eluted phages from the condition 10 μg/mL round 1 direct trypsin elution were further selected against two concentrations of coated Gal10-His (10 and 1 μg/mL). In the same process, an off-rate wash was applied during the second round of selection.

The result from the second round of selection showed a similar or up to 10-fold lower enrichment compared to the first round. The 24 hours off-rate wash resulted in a 10-fold lower output titer as compared to the condition without off-rate wash, with an exception in the case of the X3 library. One master plate was prepared for each library, including clones from the first and the second round of selection with and without off-rate selections.

### Screening Binding of the CDR2 Randomized Clones

### Production of the Fab Periplasmic Extracts

From the eluted phages of the first and second rounds of selection (with or without off-rate wash), single clones were generated and resulted in the creation of a total of four master plates (one master plate per library, with colonies selected from the different conditions as illustrated in Table 9).

TABLE 9

| | | | | Selected | Off-rate wash |
|---|---|---|---|---|---|
| | | Round | | for | (10-fold |
| Plate | | of | | Gal10 at | excess of |
| number | Library | selection | Elution | (μg/mL) | Gal10-His) |
| MP20 | X3 | 1-2 | Trypsin | 10 | 24 h |
| MP21 | X4 | 1-2 | Trypsin | 10 | 24 h |
| MP22 | X5 | 1-2 | Trypsin | 10 | 24 h |
| MP23 | X6 | 1-2 | Trypsin | 10 | 24 h |

Overview table of the Master plate (MP) generated after the selection campaign against human Gal10-His (CDR2 randomization campaign).

A total of four master plates were generated after the first and second rounds of selection against 10 μg/mL of human Gal10 with or without off-rate wash performed with a 10-fold excess of the antigen, elution method with trypsin.

From these master plates, periplasmic extracts were generated (Fab) and their binding capacities to human Gal10 were analyzed by SPR.

Screening of the Fab Periplasmic Extracts Generated from the CDR2 Randomization Campaign

The binding properties of the Fabs (periplasmic extract) to human Gal10-His were analyzed by SPR with a Biacore 3000 instrument. For this purpose, the diluted periplasmic extract was applied on a CM5 chip coated with human Gal10-His. As a positive control, a 20 nM injection of the purified g7B07 clone in the human Fab backbone was included at the beginning and end of the run. During the screening, only the dissociation (off-rate) of the Fab could be determined since the effective concentration of the Fab was unknown and can significantly vary from clone to clone.

Among the 376 clones that have been screened on SPR, most of the screened periplasmic extracts showed a higher dissociation rate than the clone g7B07 (off-rate=6.9E-03 1/s).

TABLE 10
Screening of the Fab periplasmic extracts generated from the CDR2
randomization campaign using SPR technology. Clones with similar or
better off rate as compared to the control g7B07 were highlighted in
bold (column “Fold off rate”). The randomized part of the CDR2
sequence is underlined.

| | | | Fold off-rate | | |
|---|---|---|---|---|---|
| | | | compared to | Rmax | Part of CDR2 |
| Clone | Library | kd (1/s) | g7B07 | (RU) | randomized |
| g20H09 | X3 | 8.8E−03 | 1.3 | 322 | IKWHRIT |
| | | | | | (SEQ ID NO: 38) |
| g20F04 | X3 | 1.6E−02 | 2.3 | 180 | IKHQVIT |
| | | | | | (SEQ ID NO: 39) |
| g20B05 | X3 | 2.3E−02 | 3.3 | 369 | IKPVGIT |
| | | | | | (SEQ ID NO: 40) |
| g21B01 | X4 | 2.6E−02 | 3.8 | 146 | ISGALT |
| | | | | | (SEQ ID NO: 41) |
| g21F11 | X4 | 3.5E−02 | 5.1 | 84.2 | IHTPMT |
| | | | | | (SEQ ID NO: 42) |
| g21C06 | X4 | 3.6E−02 | 5.2 | 61.7 | IKRPWT |
| | | | | | (SEQ ID NO: 43). |
| g22C03 | X5 | 2.0E−02 | 2.9 | 425 | IKLGTFT |
| | | | | | (SEQ ID NO: 44) |
| g23H09 | X6 | 2.7E−03 | 0.4 | 424 | IAQFQHWT |
| | | | | | (SEQ ID NO: 45) |
| g23E05 | X6 | 1.3E−02 | 1.8 | 624 | IRTFEHHT |
| | | | | | (SEQ ID NO: 46) |
| g23H11 | X6 | 2.5E−02 | 3.6 | 373 | ISKMPHLT |
| | | | | | (SEQ ID NO: 47) |
| g23G11 | X6 | 1.8E−02 | 2.6 | 211 | IQKTIVPT |
| | | | | | (SEQ ID NO: 48) |
| g7B07 | Ctrl | 6.9E−03 | 1.0 | 717 | IKNGGGIT |
| (mean) | | | | | (SEQ ID NO: 49) |

The binding capacity of the Fab periplasmic extract was analyzed by SPR technology using a Biacore 3000. Briefly, diluted periplasmic extracts were injected on CM5 chip coated with 2500 RU of Gal10-His. The amplitude of the binding (Rmax), the dissociation (off-rate) of each clone, as well as the off-rate fold change compared to the controls (g7B07 and g7B07_N53A) are indicated. Clones g20H09 and g23H09 had similar or better off rate compared to the control g7B07 (Table 10). The randomized part of the CDR2 sequence is underlined (Table 10).

A limited number of clones, isolated from the four different CDR2 randomization libraries, showed appropriate binding properties (Table 10). Among this panel, the clone g23H09, isolated from the X6 library, was the unique clone showing a better off-rate than the parental clone 7B07 (2.5-fold better off-rate). With an off-rate equal to 8.8E-03 1/s, the clone 20H09, isolated from the X3 library, showed a similar off-rate compared to clone g7B07. However, the randomized part of the CDR2 of the clone g20H09 (WHR) and g23H09 (AQFQHW) showed one possible liability, represented by the presence of one Tryptophan, which could potentially oxidize under influence of light.

Finally, with a 1.8-2.3-fold higher off-rate compared to g7B07, the clones 23E05 (X6 library) and 20F04 (X3 library) showed a binding property close to the parental clone. None of the clones isolated from the X4 and X5 libraries showed appropriate binding properties.

Summary of Results from Example 1

Seven clones from example 1 were selected for further characterization (Table 11). These clones were selected based on their binding capacity (BLI or SPR binding), epitope characterisation (Competition ELISA) and VH and VL sequences (Table 11).

TABLE 11
Characteristics of the scFv/Fab clones selected
for germlining and reformatting in human Fab

| | | Format of the | kd, fold compared | |
|---|---|---|---|---|
| | | Perisplasmic anti- | to g7B07 | Epitope |
| Clones | Campaign | GAL10 binders | (BLI or SPR) | binning |
| g20H09 | CDR2 | Fab | 0.8 | Not tested |
| | randomization (×3) | | | |
| g23H09 | CDR2 | Fab | 2.5 | Not tested |
| | randomization (×6) | | | |
| 18C06 | 7B07 epitope like | scFv | 14.3 | 7B07 bin |
| 24F02 | Heavy chain | Fab | 1.6 | 7B07 bin |
| | shuffling | | | |
| 18G12 | 7B07 epitope like | scFv | 2.8 | 7B07 bin |
| 18G07 | 7B07 epitope like | scFv | 7.1 | 7B07 bin |
| 18E04 | 7B07 epitope like | scFv | 7.2 | 7B07 bin |
| 7B07 | Control | Fab | 1.0 | / |

### Example 2: Germlining, Reformatting and Production of the Selected Clones in the Human Fab Backbone

Before the seven selected clones were further characterized, the clones were humanized and re-cloned into a human Fab backbone.

### Germlining of the Selected Clones Via CDR Grafting Approach

To reduce the immunogenicity of the selected anti-Gal10 clones, the germlining of the variable regions (VH and VL) was initiated through complementarity determining region (CDR) grafting into the closest human germline framework (FW). The human germline sequence with the highest identity to the V-region of the selected clones was identified.

The variant 24F02 was further engineered to remove a potential deamidation site (pos53_CDR2_VH) located at exactly the same position as g7B07. For this reason, the N position 53 was mutated to A.

As the CDR2 randomization libraries were built using the DNA of the variable domain of the heavy chain of the germlined variant of 7B07, all the generated clones (g20H09 and g23H09) were already humanized.

### Example 3: Characterization of the Binding of Germlined Clones in the Human Fab Backbone

Characterization of the Binding Properties of the Selected Fab Clones from the Three Discovery Campaigns on SPR.

The binding properties of the selected Fab clones to human Gal10 were analyzed via a capture method established on a Biacore 3000 instrument. Two concentrations of the selected Fab clones were tested on human Gal10-His immobilized on a CM5 chip coated with a monoclonal anti-His. As controls, the clone g7B07 and g7B07_N53A were injected at the beginning and the end of the run. Briefly, a CM5 chip was coated with a monoclonal anti-His antibody (4000 RU), then a fixed concentration of the human Gal10-His (25 μg/mL) was captured on the anti-His chip before receiving two concentrations of the clones in the human Fab backbone.

The germlined variants of the clones identified in Table 11 showed similar or better off-rate and affinity than clone g7B07 (see Table 12). Among this panel, the clones isolated from the 7B07 epitope campaign showed the best off-rate with a 6.2 to 9.8-fold better off-rate than clone g7B07.

Germlined (g24F02) and the engineered version (g24F02_N53A) of the initial clone 24F02 demonstrated higher off-rates in comparison to the initial off-rate (5.86E-05 1/s) observed during the earlier screening campaign on BLI. The two Fabs showed highly similar binding capacity, demonstrating that the removal of the deamidation site found at position 53 does not impact g24F02's binding properties, unlike g7B07 which showed a marked reduction in binding capacity in the engineered variant g7B07_N53A.

With an on-rate between 7.1-8.3 E+05, and off-rate equal to 1.6E-03 1/s, and an affinity between 2.0-2.3 nM, these clones showed 1.6 to 1.9-fold better on-rate, a 2.8-fold better off-rate, and a 4.7-fold better affinity than clone g7B07.

Finally, the two clones isolated from the CDR2 randomization campaign showed distinct binding properties, in line with the screening data. The clone g23H09 showed a similar on-rate, a 2.8-fold better affinity (3.9 nM), and a 1.8-fold better off-rate compared to clone g7B07. The clone g20H09 showed a 3-fold higher dissociation rate, but similar affinity.

All the clones showed better affinity and off-rate compared to the engineered variant g7B07_N53A (kd 7.8E-02 1/s, KD 143.5 nM).

TABLE 12
The binding properties of the selected
germline clones from Table 11.
Characterization of purified Fab (2 concentrations)

| | Rmax | ka | | KD | kd-fold drop |
|---|---|---|---|---|---|
| Constructs | (RU) | (1/Ms) | kd (1/s) | (nM) | vs g7B07 |
| g18G12 | 289 | 1.3E+05 | 7.6E−04 | 5.7 | 6.2 |
| g18G07 | 616 | 2.6E+05 | 6.5E−04 | 2.5 | 7.2 |
| g18E04 | 803 | 3.2E+05 | 7.4E−04 | 2.3 | 6.3 |
| g18C06 | 815 | 4.1E+05 | 4.8E−04 | 1.2 | 9.8 |
| g23H09 | 1037 | 6.5E+05 | 2.5E−03 | 3.9 | 1.8 |
| g20H09 | 797 | 8.6E+05 | 1.5E−02 | 17.5 | 0.3 |
| g24F02 | 745 | 8.3E+05 | 1.6E−03 | 2.0 | 2.8 |
| g24F02_N53A | 751 | 7.1E+05 | 1.6E−03 | 2.3 | 2.8 |
| g7B07 (mean) | 698 | 4.2E+05 | 4.7E−03 | 11.1 | 1.0 |
| g7B07_N53A | 178 | 6.4E+05 | 7.8E−02 | 143.5 | 0.06 |
| (mean) | | | | | |

### Example 4: Stability Study of the Selected Clones

To investigate issues similar to that of clone g7B07, where after stress testing a deamidation site in the CDR2 of the variable domain of the heavy chain induced a clear drop in binding and potency, the stability of the selected clones was analyzed.

For this purpose, a temperature stress study was performed. In this set-up, stressed samples were incubated for two weeks at 37° C. before being analyzed side-by-side with non-stressed samples (TO) for binding, potency (CLC dissolution), and post-translational modification (focused on the CDR2 sequence). The tested clones showed different starting concentrations (3.2-7.3 mg/mL).

### Analysis of the Binding Capacity of the Temperature Stressed Samples on SPR

The binding properties of the seven selected clones following two weeks of temperature stress was analysed using an optimized capture method on a Biacore 3000 instrument. The binding properties of the stressed samples were determined, compared to the calibration point, and expressed as a percentage of Relative Activity (% RA).

Briefly, a capture method was set-up on a Biacore 3000 instrument. For this purpose, a CM5 chip was coated with a monoclonal anti-His antibody (4000 RU), then a fixed concentration of the human Gal10-His (25 μg/mL) was captured on the anti-His chip before receiving duplicate injections of the calibrators, QC samples and temperature stressed samples of each clone. The slope of each injected sample was then calculated using the BIA evaluation software. Concentrations of the QC samples and temperature stressed samples were back-calculated by interpolating the obtained slopes of these samples to the calibrator curve. Obtained values were within +/−20% of the nominal concentration of the tested samples and are expressed as average relative accuracy (avg % RA) (Table 13).

TABLE 13
Analysis of the binding properties of the
temperature-stressed samples of the selected
clones isolated from the three discovery campaigns.

| | | % Relative Activity | |
|---|---|---|---|
| | | (RA) on Biacore | |
| Clones | From | T0 | T2W |
| g18C06 | 7B07-epitope like | 108 | 106 |
| g18E04 | 7B07-epitope like | 100 | 74 |
| g18G07 | 7B07-epitope like | 101 | 33 |
| g18G12 | 7B07-epitope like | 99 | 95 |
| g20H09 | CDR2 random (×3) | 110 | 86 |
| g23H09 | CDR2 random (×6) | 98 | 78 |
| g24F02 | Heavy chain shuffling | 101 | 175* |
| g24F02_N53A | Heavy chain shuffling | 98 | 99 |

Clones g18G12, g18C06, and g24F02_N53A showed the best stability after two weeks at 37° C., with a similar binding capacity as the non-temperature stressed samples (95%, 106%, and 99% RA respectively) (Table 13). In addition, the non-engineered variant of g24F02 did not show any loss in binding after two weeks of incubation at 37° C., demonstrating that deamidation at position 53 does not affect its binding property.

Clones g23H09 and g20H09 showed a reduced binding capacity after incubation at 37° C., which resulted in 78% and 86% RA after two weeks respectively. However, this 14% and 22% drop in binding could be attributable to assay variation.

The temperature stress of samples containing g18G07 and g18E04 showed a clear drop in binding (33% and 74% RA), highlighting their instability after two weeks at 37° C.

### Analysis of the Post-Translational Modifications (Focused on CDR2_VH)

After two weeks of temperature stress, the generated samples were analyzed for post-translational modification, which focused on the CDR2 of the heavy chain of these clones.

Most of the tested clones showed a relatively low percentage of modification after the two weeks of incubation at 37° C. Clones g18C06, g20H09 and g23H09 showed the lowest amount of modification, with respectively 3.1%, 2.4%, and 8.4% of modification in the sequences that were analyzed (Table 14).

In line with the reduction in binding activity after the incubation at 37° C., clone g18G07, and to a lesser extent clone g18E04, showed post-translational modifications (Table 14). Clone g18E04 showed 11.7% modification after two weeks of incubation at 37° C., mainly due to deamidation of the Asn position 52, which could explain its reduced binding capacity (Tables 13 and 14). The non-stressed sample of clone g18G07 showed 20.2% modification, which was increased up to 63% of modification after two weeks of incubation at 37° C., mostly due to deamidation of the Asn at position 53 (58.8%), explaining the loss in the binding capacity of the temperature stressed samples.

The non-stressed sample of clone g18G12 showed a 30% deamidation (Asn at position 54), which was not affected by the two weeks of incubation at 37° C. The deamidation of this clone most likely occurred during the production process (6 days at 37° C.).

TABLE 14
% Relative amount of modification
Post-translational modifications (PM focus CDR2_VH) of the temperature stress
samples of the selected anti-Gal10 clones. Only a part of the FW2 and the CDR2
sequences of the VH of the selected clones are depicted in the table because
post-translational modification was observed in the FW2, mostly with the W at
position 47. The underlined residues represent CDR2 (according to Kabat numbering).

| | | Modified | | | |
|---|---|---|---|---|---|
| Clones | Modification | AA site | T0 | T2W | FW2/CDR2 sequences of the VH |
| g18C06 | Non-modified | / | 98.0 | 96.9 | GLEWVSGINSGDGSSVK |
| | Deamidation | N52 | 0.7 | 0.8 | (SEQ ID NO: 50) |
| | Dioxidation | W47 | 1.3 | 2.3 | |
| g18E04 | Non-modified | / | 92.3 | 88.3 | GLEWVSAINSGGGTYYADTVK |
| | Deamidation | N52 | 3.5 | 7.2 | (SEQ ID NO: 51) |
| | (IsoAsp) | | | | |
| | Deamidation | N52 | 2.0 | 2.9 | |
| | (Asp) | | | | |
| | Succinimide | N52 | 0.3 | 0.3 | |
| | Dioxidation | W47 | 1.9 | 1.4 | |
| g18G07 | Non-modified | / | 79.8 | 37.0 | GLEWVSSIINGGESTTYRDSVK |
| | Deamidation | N53 | 10 | 43.2 | (SEQ ID NO: 52) |
| | (IsoAsp) | | | | |
| | Deamidation | N53 | 3.7 | 15.6 | |
| | (Asp) | | | | |
| | Succinimide | N53 | 2.8 | 1.7 | |
| | Dioxidation | W47 | 1.8 | 1.1 | |
| | Unknown | ? | 1.9 | 1.6 | |
| g18G12 | Non-modified | / | 61.5 | 62.8 | GLEWVSSISWNGQNTYYADTGK |
| | Deamidation | N54 | 29.8 | 28.9 | (SEQ ID NO: 53) |
| | (Asp) | | | | |
| | Succinimide | N54 | 8.6 | 8.4 | |
| g20H09 | Non-modified | / | 98.9 | 99.3 | GLEWVSAIKWHRITYYADSVK |
| | Dioxidation | W47 | 1.7 | 1.7 | (SEQ ID NO: 54) |
| | Isomerization | D61 | 1.1 | 0.7 | |
| g23H09 | Non-modified | / | 95.8 | 91.6 | GLEWVSAIAQFQHWTYYADSVK |
| | Oxidation | W57 | 1.9 | 3.4 | (SEQ ID NO: 55) |
| | Dioxidation | W47 | 1.2 | 1.4 | |
| | Isomerization | D62 | n.d | 0.3 | |
| | Unknown | Q53 or | 1.2 | 3.1 | |
| | | Q55 | | | |

### Analysis of the Efficacy of the Temperatures Stressed Sample to Dissolve Recombinant CLC

The ability of the temperature-stressed samples of the selected clones in human Fab backbone to dissolve recombinant CLC was tested.

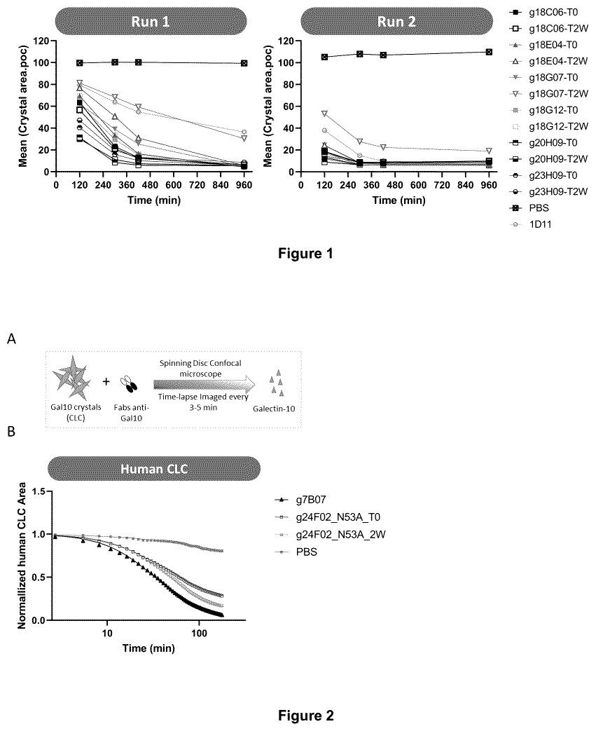

Clone 1D11 (anti-human specific) was included as a reference to correct for inter-assay variation. The dissolution of CLCs was monitored over time using an InCell 2200 analyzer after 2, 5, 7, and 16 hours of incubation (FIG. 1). Briefly, antibodies were tested at 250 μg/mL (n=4) in two independent experiments and images captured at 2, 5, 7, and 16 h after antibody addition. The images were segmented using an algorithm developed to detect individual crystals and the total crystal area per well. In this set-up, 1 μL of the samples (TO and T2W) diluted at 1.5 mg/mL was applied to recombinant CLCs formed with 1 μg of Gal10.

TABLE 15
The mean % crystal area dissolved/well after
the incubation with each of the clones.

| | CLC dissolution potency | | | | | | | |
|---|---|---|---|---|---|---|---|---|
| | Range crystal (μm) | | | | | | | |
| | 10-20 | | 5-10 | | 10-20 | | 5-10 | |
| | Run 1_2 h | | Run 2_2 h | | Run 1_5 h | | Run 2_5 h | |
| Clones | T0 | 2 W | T0 | 2 W | T0 | 2 W | T0 | 2 W |
| g18G12 | 37.9 | 30.9 | 82.6 | 80.5 | 70.4 | 66.3 | 93.5 | 92.0 |
| g18G07 | 37.9 | 18.6 | 75.6 | 46.7 | 61.0 | 31.7 | 91.4 | 72.4 |
| g18E04 | 30.6 | 23.3 | 81.7 | 75.7 | 66.5 | 49.3 | 93.8 | 92.1 |
| g18C06 | 36.6 | 43.4 | 81.4 | 81.4 | 76.8 | 78.8 | 92.1 | 91.4 |
| g23H09 | 59.6 | 52.9 | 87.9 | 85.6 | 84.1 | 80.6 | 94.0 | 90.9 |
| g20H09 | 68.6 | 70.0 | 91.0 | 86.7 | 91.4 | 88.8 | 93.7 | 93.6 |
| 1D11 | 20.5 | / | 62.1 | / | 36.2 | / | 85.0 | / |

The assays were performed with different sizes of CLCs, varying from 5-10 μm (Run 2) up to 10-20 μm (Run 1). It was observed that the crystal size had an impact on the efficacy of the Fab. In particular, a CLC with a size between 10-20 μm allowed the best discrimination between the clones.

Clones g20H09 and g23H09 were the most potent clones in the panel (FIG. 1 and Table 15). These clones were capable of dissolving 59.6% and 68.6% of the recombinant CLCs within 2 hours (Run 1). In contrast, the other clones showed a dissolution of 30.6% to 37.9% after 2 hours (Run 1). Overall, after 5 hours of incubation, most clones (except g18G07 and 1D11) were able to solubilize ˜90% of the CLCs.

The positive control, clone 1D11, showed the lowest potency to dissolve the crystals with 20% and 36% CLC dissolution after 2 and 5 hours of incubation respectively.

In line with the other stability results (binding and post-translational modifications), the temperature stressed samples of clones g18C06, g20H09, g23H09 and g18G12 showed similar potency as the non-stressed samples. In line with the reduced binding capacity of the temperature stressed samples of clone g18G07 and g18E04, these samples showed a reduced efficacy to dissolve the recombinant CLCs.

The potency of g24F02_N53A to dissolve recombinant CLC was analyzed with a spinning disk confocal microscope. Briefly, the humanized Fab fragments were incubated with pre-formed CLC and dissolution of the crystal was monitored over time.

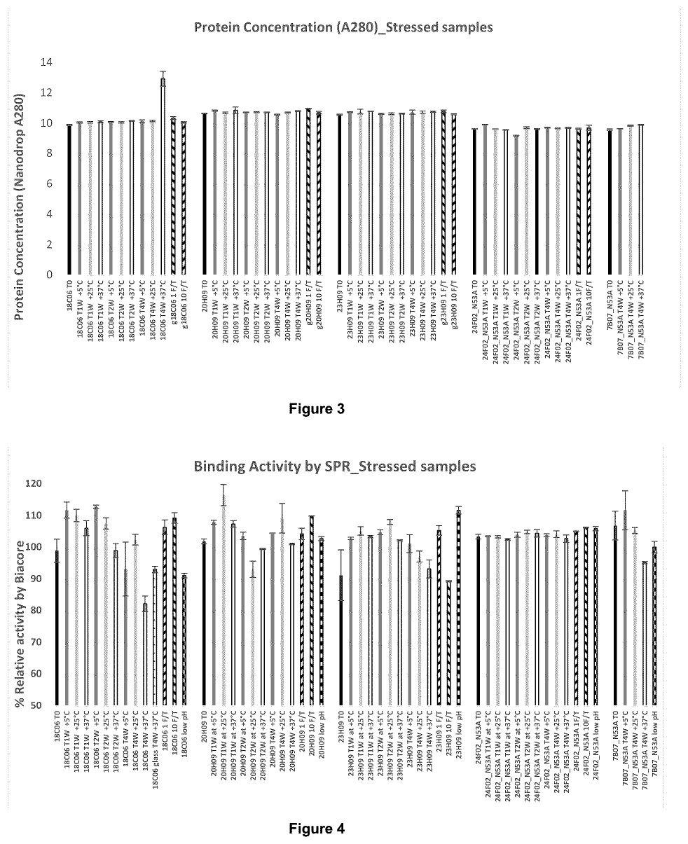

In line with the binding data, the temperature stressed samples (2 weeks at 37° C.) showed similar efficacy when compared to the non-stressed samples (FIG. 2 and Table 16). This highlights the stability of this clone for two weeks at 37° C. In fact, the tested samples from this clone showed a 50% CLC dissolution within 43-46 minutes (Table 16). Clone g24F02 showed a similar, but slightly lower, potency as compared to g7B07 in both of the non-stressed and temperature samples.

TABLE 16
The EC50 and EC90 values of the temperature
stressed samples (T0 and T2W at 37° C.).
The calculations were made using a non-linear
regression (log(agonist) vs. response Variable slope
(four parameters)) and reported in the table. The
results are combinations of one experiment
where each well was monitored in six replicates.

| | CLC dissolution | |
|---|---|---|
| | assay (Time Lapse) | |
| | 50% dissolution | 90% dissolution |
| Clones (hFab) | (min) | (min) |
| g7B07 | 34.44 | 135.1 |
| g24F02_N53A_T0 | 43.7* | >170 |
| g24F02_N53A_2W | 45.95* | >170 |

*Partial curve

### Characterization of the Binding Site of the Selected Clones Via Epitope Binning Using ELISA

An epitope binning analysis of the humanized Fab clones was carried out in comparison with the humanized Fab of g7B07.

For this experiment, a sub-optimal concentration of the biotinylated clone g7B07-humanized Fab was added to the Gal10 coated plate and pre-incubated with the selected clones. The percentage competition with g7B07 was then determined (Table 17). Motavizumab (Mota) in a human Fab backbone was used as a negative control (0% competition). Clone g7B07 was used as the positive control for competition and was therefore set as the 100% competition value. The anti-human specific clone 1D11 (binding to Tyrosine 69 residue) was included in the tested panel as a negative control for competition with 7B07 for Gal10 binding because it was known that clone 1D11 binds to the opposite side of Gal10 (including Tyrosine 69).

TABLE 17
Epitope binning of the selected anti-Gal10 clones against g7B07-human
Fab-biotinylated on ELISA. The percentage of competition against
g7B07-hFab-Biot was measured by using the OD value of the
negative control Motavizumab as 0% competition and the OD value
of non-biotinylated clone g7B07 as 100% competition.

| | Epitope binning | |
|---|---|---|
| Clones | Mean OD | % Competition |
| (hFab) | value | against g7B07-Biot |
| g18C06 | 0.05 | 105.3 |
| g24F02 | 0.26 | 96.5 |
| g24F02_N53A | 0.20 | 99.0 |
| g20H09 | 0.33 | 93.2 |
| g23H09 | 0.31 | 94.3 |
| g7B07 | 0.18 | 100.0 |
| Mota-hFab | 2.51 | 0.0 |
| 1D11-hFab | 1.55 | 41.0 |
| Buffer | 2.37 | 6.0 |

The results confirmed that the selected clones competed with g7B07 for Gal10 binding (Table 17).

Characterization of the Binding Properties of the Selected Fab Clones from the Three Discovery Campaigns on SPR

The binding properties of the selected clones to human and cynomolgus Gal10 was analyzed via a capture method established on a Biacore 3000 instrument.

For this purpose, two approaches were used. In the first, two concentrations of the selected clones were applied on cynomolgus (WGS isoform) Gal10-His capture on a CM5 chip coated with a monoclonal anti-His antibody. The second approach involved a serial dilution that was applied on human or cynomolgus (WGS isoform) Gal10-His in the same set-up.

In the first phase, the cynomolgus cross reactivity of the panel was tested via the injections of two concentrations on captured cynomolgus Gal10 (WGS isoform).

Clone g7B07 and its engineered variant g7B07_N53A showed weak binding to the WGS isoform of the cynomolgus Gal10 (KD 69 nM for g7B07-hFab versus 1.5 nM for g7B07-mIgG1) (Table 18).

Clone g20H09 showed a poor binding capacity to the cynomolgus antigen, whereas the clone g18C06 showed no binding (Table 18). However, the clones g23H09, g24F02 and its engineered variant g24F02_N53A showed good cynomolgus cross-reactivity (Table 18).

Further characterization of the cynomolgus cross-reactivity of these clones highlighted g24F02_N53A and g23H09. Indeed, these two clones showed a 1.4 nM and 5.34 nM affinity on human Gal10 and 8.0 and 9.9 nM affinity on cynomolgus Gal10, respectively. In addition, these two clones (g23H09 and g24F02_N53A) showed a 1.7-fold and 6.3-fold better affinity and 1.9-fold up to 3.5-fold better off-rate than clone g7B07. The mutation of the Asn at position 53 in the CDR2 of the g24F02_VH did not translate to reduced binding to human or cynomolgus Gal10, and similar affinity and off-rates were observed.

In line with the screening data, clone g18C06 showed the best affinity (1.27 nM) and off-rate (4.9E-04 1/s) of the tested panel on human Gal10, but no binding to its cynomolgus counterpart.

The randomization of the 6 amino acids of the CDR2 of the g7B07_VH resulting in the generation of g23H09 showed an increase in cynomolgus cross-reactivity compared to the parental clone. However, the clone g20H09, isolated from a similar discovery campaign from which 3 amino acids of the CDR2 had been randomized, did not show this gain of cynomolgus cross-reactivity, demonstrating that key amino acids have been introduced in the CDR2 of clone g23H09, leading to an increased binding to cynomolgus Gal10.

TABLE 18
Affinity determinations as determined by SPR.

| | Affinity determination (SPR) | | | | | | | |
|---|---|---|---|---|---|---|---|---|
| | Human Gal10-His | | | | Cynomolgus Gal10-His (WGS isoform) | | | |
| Clones | ka | kd | Amplitude R0 | KD | ka | kd | Amplitude R0 | KD |
| (hFab) | (1/Ms) | (1/s) | (RU) | (M) | (1/Ms) | (1/s) | (RU) | (M) |
| g7B07 | 6.7E+05 | 6.0E−03 | 607 | 9.08 | 1.9E+06 | 1.3E−01 | 64 | 69.5 |
| g7B07_N53A | 5.4E+05 | 7.9E−02 | 119 | 145.00 | 5.1E+05 | 2.1E−01 | 30 | 417.0 |
| g23H09 | 5.9E+05 | 3.1E−03 | 722 | 5.34 | 9.5E+05 | 9.5E−03 | 589 | 9.9 |
| g18C06 | 3.8E+05 | 4.9E−04 | 598 | 1.27 | no | no | no | no |
| | | | | | binding | binding | binding | binding |
| g24F02 | 1.1E+06 | 1.6E−03 | 644 | 1.49 | 1.4E+06 | 7.8E−03 | 566 | 5.4 |
| g24F02_N53A | 1.3E+06 | 1.8E−03 | 647 | 1.43 | 1.1E+06 | 8.8E−03 | 544 | 8.0 |

### Summary of Example 4 Results

In view of all of the parameters that were analysed, four clones were selected for further characterization:

g18C06: Identified during the “7B07 epitope campaign”, showed the best off-rate on human Gal10 (4.9E-04 1/s), a good potency to dissolve recombinant CLCs (76.8% after 5 hours) and appropriate stability (3.1% of post-translational modification, binding and potency unchanged after two weeks at 37° C.). Despite a similar binding region on Gal10 as the g7B07, this clone showed no binding to cynomolgus Gal10.

g23H09: This variant of g7B07, in which 6 residues in the CDR2 of the heavy chain were randomized, showed similar binding properties as the parental clone. Besides suitable stability (binding and potency unaffected after two weeks of incubation at 37° C.), this clone showed the second-best efficacy to dissolve recombinant CLC (84.1% after 5 hours). It also showed clear cynomolgus cross-reactivity, with an affinity to the WGS isoform of 9.9 nM, which is 7-fold better than g7B07 and would allow use of g23H09 for toxicology testing in the cynomolgus monkey.

g24F02_N53A: The unique clone identified from the “Heavy chain” shuffling of g7B07 met all the acceptance criteria. A possible deamidation site, similar to the one found in g7B07, was mutated to an alanine. The potency and binding capacity was unchanged even after two weeks at 37° C. With an 8 nM affinity to cynomolgus Gal10, this clone showed good cynomolgus cross-reactivity, which would allow toxicology testing in cynomolgus monkey.

g20H09: This variant of g7B07, in which 3 residues in the CDR2 of the heavy chain were randomized, clearly showed a lower binding capacity than the parental clone. Despite suitable stability and the best efficacy to dissolve the Gal10 crystals (91.4% after 5 hours), this clone exhibited weak cross-reactivity to cynomolgus Gal10.

TABLE 19
Summary characteristics for clones: g23H09,
g24F02_N53A, g18C06 and g20H09.

| Clone | g23H09 | g24F02_N53A | g18C06 | g20H09 |
|---|---|---|---|---|
| Discovery | CDR2 | Heavy chain | 7B07 | CDR2 |
| campaigns | randomization | shuffling | epitope | randomization |
| | (N6) | | like | (N3) |
| Affinity (human/ | 5.34 nM/ | 1.4 nM/ | 1.27 nM/ | 17.5 nM/ |
| cynomolgus_WGS) | 9.9 nM | 8 nM | no | 22 nM |
| via SPR | | | binding | |
| Off rate (human/ | 3.1E−03/ | 1.8E−03/ | 4.9E-04/ | 1.5E−02/ |
| cynomolgus_WGS) | 9.5E−03 | 8.8E−03 | no binding | 4.6E−02 |
| kd1/s via SPR | | | | |
| Epitope on human | Overlapping g7B07 | | | |
| Gal10 | | | | |
| Stability study | Binding and potency unchanged/>91% of non-modified | | | |
| (binding/potency/ | CDR2 (not tested for g24F02_N53A) | | | |
| post translational | | | | |
| modification) after 2 | | | | |
| weeks at 37° C. | | | | |
| Potency to dissolve | | | | |
| rec CLC | | | | |
| % dissolution after 5 | 84.10% | n.t | 76.80% | 91.40% |
| hours incubation | | | | |
| 50% dissolution | n.t | 43.7 minutes | n.t | n.t |
| reached in: | | | | |
| Potential liabilities | None | Correction of | None | None |
| | | deamidation | | |
| | | site (pos | | |
| | | 53_VH) | | |
| % identity to human | 98.8 | 100 | 100 | 98.8 |
| germ V-region | | | | |
| (closest variant) | | | | |
| % identity of the VH | 95%/6 aa | 88%/14 aa | 78%/27 aa | 96%/4 aa |
| compared to g7B07/ | | | | |
| number of aa differ | | | | |
| % identity of the VL | 100% | 100% | 56%/48 aa | 100% |
| compared to g7B07/ | | | | |
| number of aa differ | | | | |

### Materials and Protocols Used in Examples 1-4

### Charcot Leyden Crystal (CLC) Dissolution Assays

### Assay 1:

Purified monoclonal Fab was supplied in PBS and stored at 4° C. Protein concentrations were determined by measuring the absorption at 280 nm using the theoretical extinction coefficients.
Human CLC crystals were produced as described in Persson et al. 5. Briefly, 5 mL of purified N-terminally His-tagged human Gal10 in PBS at a concentration of 4 mg/ml was incubated overnight at room temperature with TEV protease at a protease: target ratio of 1% (g/g). The next day crystallization was induced by vortexing the solution. (15 s). CLC crystals appear within 30 minutes to 1 hour resulting in a turbid solution. This crystalline solution is then stored at 4° C. until further usage.

### Assay 2:

Purified monoclonal Fabs were supplied in PBS and stored at 4° C. Protein concentrations were determined by measuring the absorption at 280 nm using the theoretical extinction coefficients.
Antibodies were tested at 250 μg/mL (n=4) in two independent experiments and images captured at 2, 5, 7, and 16 h after antibody addition. 1D11 concentration-response curve (CRC) included (n=3) as additional positive control.
Images were segmented using an in-house algorithm to detect individual crystals and calculate the total crystal area per well.

### Protocols

### 1.1 Library Construction for the CDR2 Randomization Campaigns

### Nested PCRs

In order to generate the libraries of the CDR2 randomization campaign, two step nested PCRs were performed to amplify the VH of the clone g7B07 and introduce the randomization of 3-4-5 residues (−1 residues) and the control 6 residues. After digestion with NcoI and NheI. PCR products were ligated into PCB13 (NheI/NcoI and BstelII (to avoid self-ligation)) vectors containing the light chain of g7B07 before being electroporated into TG1 ECC.

### Two Step Nested PCRs:

### 1^{rs}t PCR:

The g7B07_V_{H}_PCB13 was used as a template, a solution at 50 ng/μL was prepared in 2× MQ water.

TABLE 20
Primers

| Primers | Sequence |
|---|---|
| >7B7NcoIs | ACTGGCGGCCCAGCCGGCCATGGCCGAAGTGCAGC |
| | (SEQ ID NO: 56) |
| >7B7NheIas | GTCAGTGCTAGCAGAGCTAACGGTG |
| | (SEQ ID NO: 57) |
| >7B7N3as | AGAGTCGGCGTAGTAGGTGATMNNMNNMNNCTTGATG |
| | GCGGACACCCAT (SEQ ID NO: 58) |
| >7B7N3s | ATCACCTACTACGCCGACTCT (SEQ ID NO: 59) |
| >7B7N5as | ACAGAGTCGGCGTAGTAGGTMNNMNNMNNMNNMNNGA |
| | TGGCGGACACCCATTCAA (SEQ ID NO: 60) |
| >7B7N4as | ACAGAGTCGGCGTAGTAGGTMNNMNNMNNMNNGATGG |
| | CGGACACCCATTCAA (SEQ ID NO: 61) |
| >7B7N5s | ACCTACTACGCCGACTCTGT (SEQ ID NO: 62) |
| >7B7N6as | ACAGAGTCGGCGTAGTAGGTMNNMNNMNNMNNMNNMN |
| | NGATGGCGGACACCCATTCAA (SEQ ID NO: 63) |

### Preparation of the First Sample Reaction (CFR Tables Below):

TABLE 21
Constituents of PCR samples

| | Volume per | | | | | |
|---|---|---|---|---|---|---|
| Reagent | reaction (μL) | x9 | | | | |
| DNA 1rst PCR | 1 | 9 | N3 | PCR1 | >7B7Ncols | >7B7N3as |
| (20 ng) | | | Library | | | |
| Oligo 5 μM | 5 | 45 | | PCR2 | >7B7Nhelas | >7B7N3s |
| (sense) | | | | | | |
| Oligo 5 μM | 5 | 45 | N4 | PCR1 | >7B7Ncols | >7B7N4as |
| (anti-sense) | | | Library | | | |
| dNTP (2 mM) | 5 | 45 | | PCR2 | >7B7N5s | >7B7Nhelas |
| Phusion Buffer | 10 | 90 | N5 | PCR1 | >7B7Ncols | >7B7N5as |
| HF (5x) | | | Library | | | |
| Phusion | 1 | 9 | | PCR2 | >7B7N5s | >7B7Nhelas |
| Polymerase | | | | | | |
| H2O (2X MQ) | 23 | 207 | N6 | PCR1 | >7B7Ncols | >7B7N6as |
| Total | 50 | 450 | Library | PCR2 | >7B7N5s | >7B7Nhelas |

Then the plate was sealed and loaded into the PCR instrument.

TABLE 22
PCR cycle conditions

| Program | ° C. | Time | | |
|---|---|---|---|---|
| 30CYC | 98 | 2 min | | |
| | 98 | 30 sec | Denaturation | 30 |
| | 55 | 20 sec | Annealing | CYCLES |
| | 72 | 15 sec | Extension | |
| | 72 | 5 min | | |
| | 4 | Standby | | |

Finally, the DNA products from the 1st PCR were isolated on agarose gel electrophoresis.

### Isolation of DNA Via Agarose Gel Electrophoresis:

1. An 0.8% agarose gel was prepared (10 wells combs (120 μL per well)).
2. Identical PCR products were pooled (8 replicates, for a total volume of 400 μL) and 80 μL of 6x Orange loading dye (Catalog R0631) was added on the mixture.
3. After polymerization, the gel was transferred to the electrophoresis system, and the tank was filled with fresh 1×TEA.
4. The Loader (Gene Ruler Mix, 20 μL) was loaded in one well.
5. Then 120 μL of cDNA was transferred in each well (3 wells in total).
6. The separation was run for at least 70 minutes at 200 Volt.
7. Proper separation of the product was assessed before to continue with the extraction of the expected DNA products (15 mL tubes).

### PCR Clean-Up:

The clean-up of the PCR product isolated from the agarose gel was performed according to the protocol provided with the NucleoSpin Gel and PCR Clean-up kit:
1. The PCR products were weighted before the addition of the NTI buffer (2 times more than 100 mg of gel, 20-30 minutes at 65° C.). The solution was left at the bench for 15 minutes (cool down) before loading on PCR Clean-up columns and centrifuged for 1 minute at 11 000 g.
2. The flow-through was discarded, 700 μL of NT3 was added to wash the membrane and the columns were centrifuged for 1 minute at 11 000 g. This step was performed twice.
3. The flow-through was discarded and the columns were centrifuged for 3 minutes at 11 000 g.
4. Finally, the PCR Clean-up columns were transferred in a 1.5 mL tube and elution was done by the addition of 20 μL of warm MQ water (70° C.).
5. After an incubation step at room temperature (1 minute), the columns were centrifuged for 1 minute at 11 000 g and eluate DNA was measured with the Nanodrop (260 nm).

### 2^{nd} PCR:

For the second nested PCR, DNA product was prepared from the 1^{rst} nested PCR to be at 10 ng/μL.

TABLE 23
Primers

| Primers | Sequence |
|---|---|
| >7B7NcoIs | ACTGGCGGCCCAGCCGGCCATGGCCGAAGTGCAGC |
| | (SEQ ID NO: 64) |
| >7B7NheIas | GTCAGTGCTAGCAGAGCTAACGGTG |
| | (SEQ ID NO: 65) |

1. Preparation of the second PCR:

TABLE 24
PCR sample constituents

| | Volume |
|---|---|
| | per |
| | reaction |
| Reagent | (μL) |
| DNA 1rst PCR (20 ng) | 1 |
| Oligo 5 μM (sense) | 5 |
| Oligo 5 μM (anti-sense) | 5 |
| dNTP (2 mM) | 5 |
| Phusion Buffer HF (5×) | 10 |
| Phusion Polymerase | 1 |
| H2O (2 × MQ) | 23 |
| Total | 50 |

2. Then the plate was sealed and loaded into the PCR instrument.

TABLE 25
PCR cycle conditions

| Program | ° C. | Time | | |
|---|---|---|---|---|
| 30CYC | 98 | 2 min | | |
| | 98 | 30 sec | Denaturation | 30 |
| | 55 | 20 sec | Annealing | CYCLES |
| | 72 | 15 sec | Extension | |
| | 72 | 5 min | | |
| | 4 | Standby | | |

3. While the PCR program was running, a 0.8% agarose gel was prepared.
4. At the end of the program, purification of the DNA products was done via electrophoresis (0.8% agarose gel) and PCR Clean up was done as described above. Finally, the DNA concentration was measured at the Nanodrop.
   Digestion of the PCR Products and the Vector Containing the g7B07 VL_{L}
1. The PCR products from the 2^{nd} nested PCR was diluted at 10 ng/μL.

TABLE 26
Digestion of PCR products

| | g7B07_VH- | g7B07_VH- | g7B07_VH- | g7B07_VH- |
|---|---|---|---|---|
| | N3 | N4 | N5 | N6 |
| DNA | 109.5 | 102.4 | 113.3 | 106.7 |
| Tango Buffer 10× | 30 | 30 | 30 | 30 |
| Ncol (10 U/μL) | 10 | 10 | 10 | 10 |
| Nhel (10 U/μL) | 10 | 10 | 10 | 10 |
| H2O (2 × MQ) | 140.5 | 147.6 | 136.7 | 143.3 |
| | 300 | 300 | 300 | 300 |

2. The solution was distributed at 50 μL in PCR tubes (6 tubes in total (300 μL)).
3. Digestion was performed for 4 hours at 37° C. on a PCR device (Thermocycler).
4. At the end of the incubation time, the PCR reactions of each library were pooled in 1.5 mL tube.
5. Purification was done via Nucleospin columns (cfr protocol above, one column per library).
6. Elution was performed with 75 μL warm 2× MQ water per column (150 μL in total for 10 μg).
7. Then the DNA concentration was measured at the Nanodrop.
   Ligation of the Digested VH into the Digested PCB13 Containing the VL

Ligation of the NcoI/NheI digested VH (N3-4-5 or 6) was done into the NcoI/NheI/BsteII digested g7B07_V_{L}PCB13.

### Preparation of the Mix was Done in 1.5 mL Tube (1 Tube Per Library).

TABLE 27
Conditions for ligation reaction

| | Volume (μL) | | | | |
|---|---|---|---|---|---|
| Reagent | N3 | N4 | N5 | N6 | Neg |
| Digested Vector 2 μg | 79.1 | 79.1 | 79.1 | 79.1 | 7.9 |
| Insert 1 μg | 17.8 | 19.8 | 19.9 | 20.4 | 0.0 |
| Buffer T4 DNA Ligase | 20 | 20 | 20 | 20 | 2 |
| (10×) | | | | | |
| T4 DNA Ligase | 5 | 5 | 5 | 5 | 0.5 |
| (5 U/μL) | | | | | |
| 2 × MQ H2O | 78.2 | 76.2 | 76.0 | 75.5 | 9.6 |
| Total | 200 | 200 | 200 | 200 | 20 |

1. The ligated products were incubated for 2 hours at room temperature or overnight at 16° C.
2. After this incubation time, extra DNA ligase (2.5 μL T4 DNA Ligase, 5 L Buffer T4 DNA Ligase in 50 μL MQ water) was added for a total volume of 250 μL.
3. Finally, the ligation was continued for 2 hours at 37° C. (WaterBath).
4. Then the DNA was purified via Nucleospin columns. For this purpose, 500 μL of NTI buffer was added to the ligated solution before proceeding with the washings steps explained earlier. To finish, elution was performed with 30 μL of warm MiliQ water per column.

### Electroporation for Final Libraries

### Day −1:

The recovery medium was transferred from −80° C. to 4° C.

### Day 0:

Four hours before the start of the experiment, the recovery medium was warmed at 37° C. (incubator) and the Gene Pulser/MicroPulser Electroporation Cuvettes were placed on ice.
1. The Electrocompetent cells (ECC, TG1) were transferred to the ice for 15 minutes.
2. Then 50 μL of ECC was added to the 30 μL of the purified ligated product (Library).
3. An extra 50 μL of ECC was added on top of the previous volume to reach a total volume of 127 μL.
4. This volume was then divided into 3 fractions and 42 μL was transferred to 3 precooled BioRad cuvettes.
5. For the negative control, a 1/10 dilution was prepared. From this pre-dilution, 10 μL was transferred in a new 1.5 ml tube. Then 20 μL of ECC was added on top of the previous volume to reach a total volume of 30 μL.
6. Electroporation was performed using the EC1 program (value should be above 4.6, BioRad Micropulser).
7. The ECC was recovered via the addition of 4 mL of pre-warmed recovery buffer (1 mL per cuvette+1 mL used to rinse the 3 cuvettes). The solution was then transferred in 15 mL tubes (4 mL in total per library).
8. The cultures were incubated for 30 minutes at 180 rpm at 37° C.
9. After incubation, a 1/1000 dilution was done for each library in 2TY medium/Ampicillin/glucose (new 15 mL tube). A 1/10 serial dilution was then performed on 96 wells plate (F bottom) to reach a 10-07 dilution.
10. Finally, 5 μL of each dilution was spotted on dedicated Petri dishes. The plates were then incubated overnight at 37° C.
11. The rest of the recovered cultures (15 mL tube with 4 mL) were used to inoculate 300 mL of 2TY medium with 2% glucose and Ampicillin (pre-warmed). The culture was then incubated at 37° C. with shaking (110 rpm) for 9 hours.

### Phage Preparation

### Phage Infection:

From the saturated library overnight cultures:
1. 4 mL were added on 400 mL of 2TY/Ampicillin/Glucose (OD value should be below 0.1) and incubated at 37° C. 120 rpm until an OD value of 0.5 was reached (around 2 hours).
2. 100 mL of this culture (100 mL per library) was transferred in a new 500 mL Erlenmeyer.
3. 20 μL of helper phages VCSM (stock 1×10¹³ phages/mL) were added and the culture was incubated for 30 minutes at 37° C. without shaking and then 30 minutes with shaking (110 rpm).
4. 100 mL of this culture was transferred to two 50 mL falcon tubes (2 per library) before being centrifuged 10 minutes at 3500 g at room temperature.
5. Each bacterial pellet (2 pellets for each library) was re-suspended in 200 ml of 2TY/Ampicillin/Kanamycin (dilution 1/1000).
6. The cultures were then incubated overnight at 28° C. with shaking (110 rpm).
   Phage Display Selections of the scFv Libraries (Montoyo+Ynigo, Kappa+Lambda) to the g7B07 Epitope

### Day 1:

### Coating:

1. A Maxisorp plate was coated with 100 μL per well of 25 μg/mL of the clone 1D11 (binding to a Gal10 region on the opposite side of the g7B07 epitope). A PBS control was included as negative control.
2. The plate was covered with a sealing tape and incubated overnight at 4° C.

### Phage Preparation:

1. Round 1: A solution containing 50 OD (1 OD=2×10⁸ cells/mL) of the glycerol stock (TG1 cells electroporated with the final library) was added to 650 mL 2TY/2% glucose/Ampicillin.
   Round 2: The overnight infections (rescues from a previous round of selection) were diluted into 15 mL of LB/Ampicillin/glucose 1/100, before to be incubated at 37° C. with shaking (110 rpm) until A600 around 0.5 (+/−2 hours) was reached.
2. When the OD value was between 0.5-0.6, the helper phage step was initiated (at this stage, the highest expression of the pili at the surface of the bacteria allows a good infection by the phage).
3. 10 μL of helper phage (VCSM13 (10¹³) stored in the freezer) was added to each falcon tube (10 mL R1-TG1). No need to mix (Phage: Bacteria ratio should be 10:1 and stock is 1×10¹³/mL).
4. The tubes were incubated 30 minutes at 37° C. without shaking (infection) before being centrifuged 15 minutes at 4800 g at room temperature (remove supernatant).
5. The pellets were resuspended (50 ml tube) in 50 mL 2TY/Ampicillin/Kanamycin (dilution 1/1000) (no glucose) in 250 mL Erlenmeyer before to being incubated overnight at 28° C. (110 rpm).

### Tg1 Inoculation:

1. 10 mL of LB medium was inoculated with a single colony of TG1 grown on a minimal salts agar plate in a 50 mL Erlenmeyer.
2. The culture was incubated overnight at 37° C. (100 rpm).

### Day 2:

### Blocking

1. After the overnight incubation, the coating plate was washed 3 times with 300 μL per well of 1×PBS-0.05% Tween via a multistepper pipette.
2. The blocking step was performed via the addition of 200 μL per well of 1×PBS 2% MARVEL using a multichannel pipette from a disposable reservoir.
3. The plate was sealed and incubated for 2 hours at room temperature while shaking at 450 rpm on a platform shaker.

### Phage Precipitation:

1. From the overnight growth (TG1 infected with helper phage) 50 mL was transferred to a new 50 ml tube and centrifuged for 15 minutes (4800 g) at 4° C. (Round 1: 2×50 mL).
2. The supernatant was collected and 40 mL was transferred to a new 50 ml tube containing 10 mL of cold 20% PEG6000/2.5 M NaCl (precipitation of the phages).
3. Then the tubes were incubated on ice for 30 minutes.
4. The precipitation of the phages was done via a centrifugation step of 15 minutes at 4800 g.
5. The supernatant was removed and the pellet was resuspended in 1 mL of sterile PBS and transferred to a new sterile 1.5 mL tube.
6. The tubes were then centrifuged for 3 minutes at max speed (table centrifuge).
7. The supernatant was collected and added to a new Eppendorf tube containing 250 μL of 20% PEG/2.5M NaCl before being incubated on ice for 15 minutes.
8. The tubes were centrifuged for 3 minutes at max speed and supernatant was removed.
9. The pellet was resuspended in PBS (round 1:500 μL per tube finally pooled; round 2: 1 mL).
10. As additional step, the solution was centrifuged for 3 minutes, max speed, to pellet the residual cell debris. Supernatant was collected (phages) into a new 1.5 mL tube to create the INPUT.
11. From this stock solution glycerol stock was made (800 μL of phages in 400 μL of 60% glycerol).
   Gal10 Capturing on the Maxisorp Coated Plate Coated with Clone 1D11:
1. The blocking buffer of the coated plate was removed by inverting the plate and tapping it on a piece of paper.
2. 100 μL per well of 5 μg/mL Gal10-His in 0.2% MARVEL was added.
3. The plate was sealed and incubated for 2 hours at room temperature while shaking at 450 rpm on a platform shaker.

### Phage Selection:

1. Round 1:10 μL of phage/selection (90 μL of PBS 0.2% MARVEL+10 μL of phages).
2. Round 2:1 μL of phage/selection (99 μL of PBS 0.2% MARVEL+1 μL of phages).
3. The blocking buffer was removed from the 96 well plates by inverting and tapping it on a piece of paper and 100 μL of diluted phages was added.
4. The plate was sealed and incubated for 2 hours at room temperature while shaking at 450 rpm on a platform shaker.
5. After 2 hours of incubation the supernatant was removed from the plate (pipet).
6. The wells were washed thoroughly (200 μL/well) (25x in total).
   a. 5 times with 200 μL of PBS/Tween 0.05%.
   Before being sealed and incubated for 5 minutes at room temperature while shaking at 450 rpm on a platform shaker.
   This step was done 4 times.
   b. Then the wells were washed 3 times with 200 μL of PBS before to proceed with the elution step.

### Elution: Trypsin:

1. The PBS was removed and elution was performed with 150 μL per well of trypsin (10 mg/mL).
2. The plate was sealed and incubated for 15 minutes at room temperature while shaking at 450 rpm on a platform shaker.
3. After this incubation time, 150 μL of the elution solution was transferred to the corresponding wells of a 96 wells V bottom plate containing in each well 7.5 μL of AEBSF (inhibitor of the trypsin). Then the solution was mixed 5 times to allowed a proper neutralization of the trypsin (pipet up and down).

### Elution: Competitive Elution:

1. Competitive elution was performed by adding 100 μL per well of 2.5 mg/mL 7B07 or Motavizumab (isotype control) in 0.2% MARVEL.
2. The plate was sealed and incubated overnight at room temperature while shaking at 450 rpm on a platform shaker.
3. After this incubation time, the elution solution was transferred to a new 96 well plate.

### Infection/Rescue:

Rescue (50 mL tubes):
1. 300 μL of TG1 cells (OD values around 0.5) was added on a 50 ml tube containing 625 μL of 2TY and 75 μL of eluted phages (trypsin elution or competitive elution).
2. The tubes were incubated for 30 minutes at 37° C., no shaking.
3. At the end of this incubation time 10 mL of LB/Ampicilin/2% glucose was added to each 50 mL tube containing the TG1/phages.
4. The tubes were then incubated overnight at 37° C. with shaking (110 rpm).

### Spotting:

1. Dilution for spotting:
   a. INPUT: Dilution series of the phages (INPUT) was prepared in 2TY for spotting (LB agar plate).
      A 12 points dilution series (1/10) was prepared in 2TY (5 μL phages in 45 μL 2TY) (10⁻¹ till 10⁻¹²).
   b. OUTPUT: Dilution series of the phages (OUTPUT) was prepared in 2TY for spotting (LB agar plate).
      A 6 points dilution series (1/10) was prepared in 2TY (5 μL phages in 45 μL 2TY) (10⁻¹ till 10⁻⁶).
2. Then 50 μL of TG1 cells (OD values around 0.5) was added to the 50 μL of the phages in the dilution plates (INPUT and OUTPUT).
3. The plate was sealed and incubated for 30 minutes at 37° C., with no shaking.
4. 5 μL of each dilution (INPUT: 10⁻⁶ till 10⁻¹²; OUTPUT: 10⁻¹ till 10⁻⁶) was spotted on a dry Petri dish (LB/Ampicillin/glucose).

### Day 3:1.

1. Presence of single colonies on spotting plates was controlled.
2. Glycerol stocks of the overnight rescues was done (1 mL of the cultures+500 μL of 60% glycerol in cryovials, stored at −80° C.).

### Master Plate Generation:

1. From appropriate rescues dilution series in 2TY was prepared.
   A 6 points dilution series (1/10) was prepared in 2TY (5 μL phages in 45 μL 2TY) (10⁻¹ till 10⁻⁶).
2. 50 μL of selected dilution (10⁻³/10⁻⁴) was transferred on Petri dishes containing solid LB agar medium with 2% glucose and Ampicillin.
3. Homogenous distribution of TG1 cells was allowed by shaking with glass beads.
4. The beads were removed, and the Petri dishes were incubated overnight at 37° C.

### Day 4:

1. After the overnight incubation single colonies were picked with p10 tips and transferred to flat bottom 96 well plates containing 100 μL of LB medium supplemented with 2% glucose and Ampicillin.
2. The plate was sealed with breathable sealing tape and incubated for 5 hours at 37° C. and 120 rpm.

### Phage Display of the Heavy Chain Shuffling and Cdr2 Randomization Campaigns

### Day 1:

### Coating (Heavy Chain Shuffling):

1. Maxisorp plate was coated with 100 μL of human Gal10-His or non-tagged human Gal10 diluted in 1×PBS:
   Round 1 and Round 2:10 μg/mL and 1 μg/mL human Gal10-His.
   Round 3 and Round 4:5 μg/mL and 0.5 μg/mL of human Gal10-His and Gal10.
   As controls, a non coating condition (PBS) and an irrelevant His-tagged protein (mCD11c-His R&D Cat n° 7987-AX) were included.
2. The plate was covered with a sealing tape and incubated overnight at 4° C.

### Coating (CDR2 Randomization):

1. Maxisorp plate was coated with 100 μL of human Gal10-His diluted in 1×PBS:
   Round 1 and Round 2:10 μg/mL and 1 μg/mL human Gal10-His, diluted in 1×PBS),
   As controls, a non coating condition (PBS) and an irrelevant His-tagged protein (mCD11c-His R&D Cat n°7987-AX) were included.
2. The plate was covered with a sealing tape and incubated overnight at 4° C.

### Phage Preparation:

1. The overnight infections (Rescues from the previous round of selection) were diluted 1/100 into 15 mL of LB/Ampicillin/glucose before being incubated at 37° C. shaking (110 rpm) until and OD value (A600) around 0.5 (+/−2 hours) was reached.
2. When the OD value was between 0.5-0.6, the helper phage step was initiated (at this stage, the highest expression of the pili at the surface of the bacteria allows a good infection by the phage).
3. 10 μL of helper phage (VCSM13 (10¹³) stored in the freezer) was added to each falcon tube (10 mL R1-TG1). No need to mix (Phage: Bacteria ratio should be 10:1 and stock is 1×10¹³/mL).
4. The tubes were incubated 30 minutes at 37° C. without shaking (infection) before being centrifuged 15 minutes at 4800 g at room temperature (remove supernatant).
5. The pellet were resuspended (50 ml tube) in 50 mL 2TY/Ampicillin/Kanamycin (dilution 1/1000) (no glucose) in 250 mL Erlenmeyer before being incubated overnight at 28° C. (110 rpm).

### Tg1 Inoculation:

1. 10 mL of LB medium was inoculated with a single colony of TG1 grown on a minimal salts agar plate in a 50 mL Erlenmeyer.
2. The culture was incubated overnight at 37° C. (100 rpm).

### Day 2:

### Blocking

1. After the overnight incubation, the coating plate was washed 3 times with 300 μL per well of 1×PBS-0.05% Tween via a multistepper pipette.
2. The blocking step was performed via the addition of 200 μL per well of 1×PBS 2% MARVEL using a multichannel pipette from a disposable reservoir.
3. The plate was sealed and incubated for 2 hours at room temperature while shaking at 450 rpm on a platform shaker.

### Phage Precipitation:

1. From the overnight growth (TG1 infected with helper phage) 50 mL was transferred to a new 50 ml tube and centrifuged for 15 minutes (4800 g) at 4° C. (Round 1: 2×50 mL).
2. The supernatant was collected and 40 mL was transferred to a new 50 ml tube containing 10 mL of cold 20% PEG6000/2.5 M NaCl (precipitation of the phages).
3. Then the tubes were incubated on ice for 30 minutes.
4. The precipitation of the phages was done via a centrifugation step of 15 minutes at 4800 g.
5. The supernatant was removed and the pellet was resuspended in 1 mL of sterile PBS and transfer to a new sterile 1.5 mL tube.
6. The tubes were then centrifuged for 3 minutes at max speed (table centrifuge).
7. The supernatant was collected and added to a new Eppendorf tube containing 250 μL of 20% PEG/2.5M NaCl before being incubated on ice for 15 minutes.
8. The tubes were centrifuged for 3 minutes at max speed and supernatant was removed.
9. The pellet was resuspended in PBS (round 1:500 μL per tube finally pooled; round 2: 1 mL).
10. As additional step, the solution was centrifuged for 3 minutes, max speed, to pellet the residual cell debris. Supernatant was collected (phages) into a new 1.5 ml tube to create the INPUT.
11. From this stock solution glycerol stock was made (800 μL of phages in 400 μL of 60% glycerol).

### Phage Selection:

1. 1 μL of phage diluted in MARVEL (495 μL of PBS 2% MARVEL+5 μL of phages) from the INPUT of the 2^{nd}, 3^{rd}, and 4^{th} round of selection was used.
2. The blocking buffer was removed from the 96 well plates by inverting and tapping it on a piece of paper and 100 μL of diluted phages was added.
3. The plate was sealed and incubated for 2 hours at room temperature while shaking at 450 rpm on a platform shaker.
4. After 2 hours of incubation the supernatant was removed from the plate (pipet).
5. The wells were washed thoroughly (200 μL/well) (25× in total).
   a. 5 times with 200 μL of PBS/Tween 0.05%.
   Before being sealed and incubated for 5 minutes at room temperature while shaking at 450 rpm on a platform shaker.
   This step was done 4 times.
   b. Then the wells were washed 3 times with 200 μL of PBS before to proceed with the elution step.

### For Off-Rate Wash Conditions:

1. 150 μL of 50-100 μg/mL Gal10-His or soluble Gal10 was added in each selected wells.
2. The plate was incubated overnight or up to 2 days at 37° C. while shaking at 450 rpm on a platform shaker.
3. After the incubation time, the wells were washed 5 times with 200 μL of PBS.

### Elution:

1. The PBS was removed and elution was performed with 150 μL per well of trypsin (10 mg/mL).
2. The plate was sealed and incubated for 15 minutes at room temperature while shaking at 450 rpm on a platform shaker.
3. After this incubation time, 150 μL of the elution solution was transferred to the corresponding wells of a 96 wells V bottom plate containing in each well 7.5 L of AEBSF (inhibitor of the trypsin). Then the solution was mixed 5 times to allowed a proper neutralization of the trypsin (pipet up and down).

### Infection/Rescue:

### Rescue (50 mL Tubes):

1. 300 μL of TG1 cells (OD values around 0.5) was added on a 50 ml tube containing 625 μL of 2TY and 75 μL of eluted phages (trypsin elution or competitive elution).
2. The tubes were incubated for 30 minutes at 37° C., no shaking.
3. At the end of this incubation time 10 mL of LB/Ampicillin/2% glucose was added to each 50 ml tube containing the TG1/phages.
4. The tubes were then incubated overnight at 37° C. with shaking (110 rpm).

### Spotting:

1. Dilution for spotting:
   a. INPUT: Dilution series of the phages (INPUT) was prepared in 2TY for spotting (LB agar plate).
      A 12 points dilution series (1/10) was prepared in 2TY (5 μL phages in 45 μL 2TY) (10⁻¹ till 10⁻¹²).
   b. OUTPUT: Dilution series of the phages (OUTPUT) was prepared in 2TY for spotting (LB agar plate).
      A 6 points dilution series (1/10) was prepared in 2TY (5 μL phages in 45 μL 2TY) (10⁻¹ till 10⁻⁶).
2. Then 50 μL of TG1 cells (OD values around 0.5) was added to the 50 μL of the phages in the dilution plates (INPUT and OUTPUT).
3. The plate was sealed and incubated for 30 minutes at 37° C., with no shaking.
4. 5 μL of each dilution (INPUT: 10⁻⁶ till 10⁻¹²; OUTPUT: 10⁻¹ till 10⁻⁶) was spotted on a dry Petri dish (LB/Ampicillin/glucose).

### Day 3:

1. Presence of single colonies on spotting plates was controlled.
2. Glycerol stocks of the overnight rescues was done (1 mL of the cultures+500 μL of 60% glycerol in cryovials, stored at −80° C.).

### Master Plate Generation:

1. From appropriate rescues dilution series in 2TY was prepared.
   A 6 points dilution series (1/10) was prepared in 2TY (5 μL phages in 45 μL 2TY) (10⁻¹ till 10⁻⁶).
2. 50 μL of selected dilution (10⁻³/10⁻⁴) was transferred on Petri dishes containing solid LB medium with 2% glucose and Ampicillin.
3. Homogenous distribution of TG1 cells was allowed by shaking with glass beads.
4. The beads were removed, and the Petri dishes were incubated overnight at 37° C.

### Production and Purification of the Anti-Gal10 Leads (Fab)

### Production of the Periplasmic Extracts

### Day 1:

### Inoculation:

1. The master plate was incubated at 37° C. with shaking (110 rpm).
2. 1 mL of 2TY/Ampicillin/0.1% glucose was added to each well of a V-shaped bottom 96 Deep well plate before to be incubated at 37° C. with shaking (110 rpm) for 15 minutes.
3. Then 10 μL of bacteria from the master plate was added to the 1 mL medium of the Deep well plate.
4. The plate was then incubated at 37° C. with shaking (110 rpm) until an OD600 value around 0.8-1.0 was reached (6-7 h).

### Induction of the Production:

1. To induce the production of the scFv or Fab, 100 μL per well of the IPTG (10 mM IPTG in 2TY+Ampicilin; final concentration in wells=+/−1 mM IPTG) was added.
2. The plate was incubated overnight at 26° C. with shaking (110 rpm).

### Day 2:

1. The day after, the 96-Deep well plate was centrifuged at 4800 g for 15 minutes in a 4° C. cooled centrifuge to pellet the bacteria.
2. The medium was removed (pour off and dry on paper).
3. The plate was sealed and stored overnight at −20° C.

### Day 3:

1. The plate was transferred from the −20° C. to −80° C. for at least 1 hour.
2. The bacterial pellet was thawed at room temperature for 30 minutes.
3. The pellet was resuspended with 110 μL of PBS per well before being vortexed for 1 minute.
4. The plate was incubated for 90 minutes at room temperature on a shaker (900 rpm).
5. After this incubation time, the bacteria was then centrifuged (4800 g, 15 minutes, 4° C.).
6. The supernatant (scFv or Fab peri) was transferred into a new V-bottom 96 wells plate.
7. Storage of the periplasmic fraction containing the scFv or Fab clones was done at −20° C.
   Recloning of the VH and VL Sequences into a Human Fab Fragment Backbone

### DNA String Design

1. Using the AbAligner software (no version number), the VH and VL were aligned and compared to the closest human germline variant.
2. The CDR regions (+1 residue before CDR1 and 3) were grafted into the FW regions of the germline variant.
3. Optimization of the nucleotide sequence for human production was done with the GeneArt® tools (Life Technologies™).
4. BsmBI restriction sites were then added on the 5′ and 3′ of the optimized amino acid sequence for recloning in pUPEX vectors.
5. Finally, the DNA strings were ordered at Life Technologies™ (Thermo Fisher Scientific™).
   Digestion of the DNA Strings with BsmBI
1. For each clone, 200 ng of VH and VL was digested with BsmBI in Tango Buffer 10× in a final volume of 20 μL.
2. The mix was prepared according to the table below and 10 μL was distributed per well according to the plate layout in a PCR plate.
3. Then the PCR plate was incubated for 2 hours at 37° C. (in Thermocycler).

TABLE 28
PCR cycle conditions

| | Volume |
|---|---|
| Reagent | (μL) |
| DNA | 10 |
| DTT 10 mM | 2 |
| Tango Buffer | 2 |
| BsmBl | 0.5 |
| MilliQ water | 5.5 |

### PCR Clean-Up of the Digested DNA

The clean-up of the digested strings was performed according to the protocol provided with the NucleoSpin Gel and PCR Clean-up kit:
1. After the 2 hours of incubation at 37° C., 80 μL of MQ water was added to each digested DNA string then 200 μL of NTI buffer was added before being transferred to a PCR Clean-up column and centrifuged for 1 minute at 11 000 g.
2. The flow-through was discarded, 700 μL of NT3 was added to wash the membrane, and the columns were centrifuged for 1 minute at 11 000 g. This step was performed twice.
3. The flow-through was discarded and the columns were centrifuged for 3 minutes at 11 000 g.
4. Finally, the PCR Clean-up columns were transferred to a 1.5 mL tube and elution was done by the addition of 25 μL of warm MQ water (70° C.).
5. After an incubation step at room temperature (1 minute), the columns were centrifuged for 1 minute at 11 000 g and eluate DNA was measured with the Nanodrop (260 nm).
   Ligation of Digested DNA with Heavy and Light Chain Vector
1. Digested VH/VL (BsmBI) were inserted on:
   a. human CH1 domain BsmBI digested vector (pUPEX86) for the VH.
   b. human Lambda BsmBI digested vector (pUPEX116.9) for the VL.
2. The ligation mixture was prepared in 1.5 mL tubes to a ratio of 1:5 (vector: insert), according to the table below.

TABLE 29
Ligation conditions

| | Volume |
|---|---|
| Reagents | (μL) |
| BsmBl digested | 6 |
| DNA (insert) (40 ng) | |
| BsmBl digested | 1 |
| pUPEX vector | |
| (10 ng) | |
| Buffer T4 DNA | 1.5 |
| Ligase (10×) | |
| T4 DNA Ligase | 1.5 |
| (5 U/μL) | |
| MQ water | 5 |
| Total | 15 |

3. The ligation mixtures were incubated at room temperature for 1 hour.

### Transformation in Top10 Competent Cells

The transformation of each ligated product into Top10 competent cells was done via heat shock.
1. After 1 hour of incubation, the ligation mix was transferred to ice for 5 minutes.
2. Top10 competent cells were thawed on ice and 40 μL of Top10 cells were added to 15 μL of each ligated product.
3. The tubes were transferred to ice for 5 minutes.
4. Transformation of the competent Top10 cells was done by heat shock: 90 seconds at 42° C. in a warm water bath.
5. The tubes were then incubated for 1-2 minutes on ice.
6. Finally, the transformed Top10 cells were transferred on Petri dishes containing solid LB medium with 2% glucose and Ampicillin (resistance gene of the vectors).
7. Homogenous distribution of the cells was done by shaking with glass beads.
8. The beads were removed, and the Petri dishes were incubated overnight at 37° C.

### Colony Picking and Sequencing

1. After one night of incubation, ligated products showed a high number of single bacteria colonies whereas no/few colonies were observed for the negative controls (empty vectors).
2. Per VH or VL, 8 single colonies were picked with p10 tips and transferred in a flat bottom 96 well plate containing 100 μL of LB medium supplemented with 2% glucose and 1/1000 Ampicillin.
3. The plate was sealed with breathable sealing tape and incubated for 5 hours at 37° C. at 120 rpm.
4. Finally, 10 μL of each clone was transferred in a sequencing plate (96 well flat bottom plate containing solid LB medium+2% glucose and 1/1000 Ampicillin).
5. The sequencing plate was sent to LGC genomic (primers p90).
6. The DNA sequences obtained from LGC genomic were analyzed with the software Clone Manager version n° 9. The sequence of each clone was aligned and compared to the ordered sequence.
7. The clones that showed similar DNA sequences as the parental sequence (DNA strings) were selected.

### Amplification and MidiPrep:

1. Per construct, inoculation of 20 μL culture in 10 mL LB medium containing 2% glucose and 1/1000 Ampicillin was performed before noon.
2. The cultures were incubated at 37° C., 120 rpm.
3. In the evening, cultures were transferred into 100 mL+1/1000 Ampicillin (no glucose as this can interfere with the sequencing of the Midiprep DNA, and pUPEX vectors do not require glucose)
4. The cultures were incubated overnight at 37° C., 120 rpm.
   MidiPrep was Performed According to the Protocol Provided with the Kit:
1. The 110 mL cultures were pelleted by centrifugation (15 minutes at 4800 g, 4° C.).
2. The supernatant was removed, pellets were resuspended (vortexed and pipetted) in 8 mL of RES buffer with RNAse.
3. 8 mL of LYS buffer was added, each tube was inverted 5 times (not vortexed) until the solution was completely blue and incubated for 5 minutes at room temperature.
4. 8 mL of NEU buffer was added and mixed until the solution became white.
5. NucleonBond Xtra Midi Columns and filters were equilibrated with 12 mL of EQU buffer.
6. The bacterial solution was gently and slowly transferred on the border of the equilibrated columns.
7. The first wash was done with 5 mL of EQU buffer and after full elution, the NucleonBond Xtra column filter was removed and a second wash was performed with
8 mL of WASH buffer.
8. The columns were then transferred to a 50 mL tube and elution was performed with 5 mL of ELU buffer.
9. Precipitation of the DNA was done by the addition of 3.5 mL of isopropanol. The tubes were vortexed and centrifuged for 30 minutes at 4800 g, 4° C.
10. The supernatant was removed and 2 mL of 70% ethanol was added.
11. Tubes were centrifuged for 30 minutes at 4800 g, 4° C.
12. The supernatant was removed, and the pellet was dried at room temperature for 1 hour.
13. Finally, 100 μL of 2×MQ water was added to resuspend the pellet, and after an incubation of 30 minutes at room temperature shaking (900 rpm), the DNA concentration was measured on Nanodrop.

### Production of the Anti-Galectin-10 Clones

### Transfection of HEK293E Cells

### Day 1:

1. HEK293E cells were seeded at 0.3E+06 cells/mL.

### Day 2:

Per anti-Gal10 clone, a 15 ml tube was prepared, containing+/−7.6 mL of pre-warmed (37° C.) Optimem medium.

| | /mL of culture | |
|---|---|---|
| DNA | 0.5 | μg |
| Optimem | 20 | μL-40 μL |
| medium | | |
| PEI | 1.5 | μL |

1. MidiPrep DNA of the VH and the VL was added at a ratio of 1:3 for a final amount of 100 μg (25 μg VH+75 μg VL).
2. Finally, 300 μg of PEI was added dropwise to the tube and shortly vortexed.
3. The mixture medium/DNA/PEI was incubated for 10 minutes at room temperature before being added to the 200 mL HEK293E cultures.
4. The cells finally were put back in the incubator (37° C., 5% CO₂, 120 rpm).
5. 4 hours after transfection, pre-warmed (37° C.) 1/20 Hypep 1510 was added to each culture.
6. After 6 days of production, all the medium for each anti-Gal10 clone was collected in 50 mL tubes.
7. The tubes were centrifuged for 10 minutes at 1000 g and 4° C. and the supernatant was transferred into a new 50 ml tube.

### Purification of the Produced Anti-Gal10 Fabs Using Capture Select IgG-CH1 Beads

### Purification of the Produced Fabs was Done Using the Capture Select IgG-CH1 Beads

### Day 1:

1. CaptureSelect IgG-CH1 beads were equilibrated by washing 3 times with PBS in a 50 ml tube and a 50% slurry in PBS was made.
2. 500 μL of the equilibrated 50% slurry beads was added per 50 mL tube containing the supernatant of the HEK cultures.
3. The tubes were incubated overnight at 4° C. on a rotor (19 rpm).

### Day 2:

1. Beads were spun down (630 g, 2 minutes, 4° C., acceleration=9, deceleration=7).
2. The supernatant was collected in a new 50 mL tube.
3. 1 mL 1×PBS was added to the beads (along the sides of the falcon); re-suspended; and transferred to the column (1 column per 100 mL of HEK culture).
4. 4 mL of 1×PBS was used to wash the tubes, before to be transferred to the column.
5. The washings steps was repeated twice.
6. The columns were washed 5 times with 5 mL of 1×PBS.
7. During the washing step, three 2 mL tubes were prepared per clone and 100 μL of neutralization solution (1 M Tris pH 8) was added to each tube.
8. For the elution, the columns were transferred into a 2 mL tube (containing the neutralization solution, and 1 mL of elution solution (0.1 M Glycine pH 3) was added. After complete elution, the columns were transferred in another 2 mL tube and elution was repeated 2 times. The tubes were properly mixed to allow a good neutralization of the elution solution.
9. Then the protein concentration of each elution fraction was determined using the Nanodrop (280 nm).
10. Buffer exchange was performed by loading 4 mL on an Amicon Ultra-4 centrifugal filter. Then the columns were centrifuged for 15 minutes at 4000 g at 4° C., the flow-through was discarded and 5 mL of 1×PBS was added to the upper chamber. The columns were centrifuged again and washed 5 times with 5 mL 1×PBS in total.
11. The protein concentration was then measured on Nanodrop and corrected with the extinction coefficient of each clone.

### Screening and Characterization of the Binding and Epitope Binning of the Anti-Gal10 Molecules by ELISA

Screening of Binding Properties of the scFv or Fab Periplasmic Extracts

### Binding ELISA

### Day 1:

1. A Maxisorp plate was coated with 100 μL per well of human Gal10-His (0.5 μg/mL, diluted in 1×PBS).
2. The plate was sealed with a sealing tape and incubated overnight at 4° C.

### Day 2:

1. The following day, the plate was washed 5 times with 300 μL per well of 1×PBS-0.05% Tween.
2. Then 300 μL per well of blocking solution (1×PBS 1% Casein) was added.
3. The plate was sealed and incubated for 2 hours at room temperature while shaking at 450 rpm on a platform shaker.
4. During the incubation time, the periplasmic extracts from the Periplasmic Master Plate (PMP) plates were diluted 1 over 5 in 1×PBS-0.1% Casein in a Microplate 96 well U-bottom plate.
5. After 2 hours of incubation with the blocking solution, the plate was washed 5 times with 300 μL per well of 1×PBS-0.05% Tween.
6. With a 200 μL multichannel pipette, 100 μL per well of each diluted scFv-periplasmic extract was transferred from the dilution plate to the coated plate.
7. The plate was sealed and incubated for 1 hour at room temperature while shaking at 450 rpm on a platform shaker.
8. During this incubation time the detection antibody was prepared (Rabbit anti-Myc HRP conjugated, 1/2000) in sufficient volume of 1×PBS-0.1% Casein.
9. After the 1 hour of incubation, the plate was washed 5 times with 300 μL per well of 1×PBS-0.05% Tween.
10. With a 200 μL multichannel pipette, 100 μL per well of the detection antibody was transferred to the coated plate.
11. The plate was sealed and incubated for 1 hour at room temperature while shaking at 450 rpm on a platform shaker.
12. 15 minutes before the end of the incubation time, TMB solution was transferred at room temperature.

13. After incubation, the plate was washed 5 times with 300 μL per well of 1×PBS-0.05% Tween.
14. With a 200 μL multichannel pipette, 100 μL per well of TMB was added and incubated for 5 minutes while shaking at 450 rpm before the reaction was stopped by the addition of 100 μL of 0.5M H₂SO₄ per well.
15. Absorbance was then measured at 450 nm (reference at 620 nm) with a Tecan instrument and Magellan software version n° 7.2.

### Competitive ELISA (Binding to 7B07 Epitope)

First, a Maxisorp plate was coated with 7B07 hIG1, where hGal10-His is captured. Then, the periplasmic extracts diluted 1/5 dilution were added. Detection was done with a rabbit anti-Myc-HRP antibody

### Day 1:

1. A Maxisorp plate was coated with 100 μL per well of 7B07_hIgG1 (3 μg/mL, diluted in 1×PBS).
2. The plate was sealed and incubated overnight at 4° C.

### Day 2:

1. The plate was washed 5 times with 300 μL per well of 1×PBS-0.05% Tween.
2. The blocking step was done via the addition of 300 μL per well of 1×PBS-1% Casein.
3. The plate was sealed and incubated for 2 hours at room temperature while shaking at 450 rpm on a platform shaker.
4. After this incubation time, the plate was washed 5 times with 300 μL per well of 1×PBS-0.05% Tween.
5. Next, 1 μg/mL of human Gal10-His was added (100 μL per well, diluted in 1×PBS-0.1% Casein).
6. The plate was sealed and incubated for 1 hour at room temperature while shaking at 450 rpm on a platform shaker.
7. During the incubation time, the 1 over 5 dilution of the selected periplasmic extract of the scFv/Fab was prepared in 1×PBS-0.1% Casein in a separate Microplate 96 well U-bottom plate.
8. After 1 hour of incubation with Gal10-His, the plate was washed 5 times with 300 μL per well of 1×PBS-0.05% Tween.
9. Then with a 200 μL multichannel pipette, 100 μL of each periplasmic extract was added to the coated plate.
10. The plate was sealed and incubated for 1 hour at room temperature while shaking at 450 rpm on a platform shaker.
11. After this incubation time, the plate was washed 5 times with 300 μL per well of 1×PBS-0.05% Tween.
12. Detection was performed via the addition of 100 μL per well of Rabbit Anti-Myc HRP conjugated diluted in 1×PBS-0.1% Casein.
13. The plate was sealed and incubated 1 hour at room temperature while shaking at 450 rpm on a platform shaker.
14. 15 minutes before the end of the incubation time, the TMB was transferred at room temperature.
15. Then the plate was washed 5 times with 300 μL per well of 1×PBS-0.05% Tween.
16. Finally, with a 200 μL multichannel pipette, 100 μL per well of TMB was added to the plate. The reaction was allowed for 5 minutes while shaking at 450 rpm, and stopped by the addition of 100 μL of 0.5 M H₂SO₄ per well.
17. Absorbance was measured at 450 nm (reference at 620 nm) with a Tecan instrument.

### Screening of Binding Properties of the Fabs

### Epitope Binning

### Day 1:

1. A Maxisorp plate was coated with 100 μL of human Gal10-His (1.25 μg/mL, diluted in 1×PBS).
2. The plate was sealed and incubated overnight at 4° C.

### Day 2:

1. The plate was washed 3 times with 300 μL per well of 1×PBS-0.05% Tween.
2. The blocking step was done via the addition of 300 μL per well of 1×PBS-1% Casein.
3. The plate was sealed and incubated for 2 hours at room temperature while shaking at 450 rpm on a platform shaker.
4. During this incubation time, the dilution of the selected Fabs was prepared at 40 μg/mL in 50 μL of 1×PBS-0.1% Casein in a Microplate 96 well U-bottom plate. The final concentration of diluted Fabs will be at 20 μg/mL.
5. After 2 hours of incubation with the blocking solution, the plate was washed 3 times with 300 μL per well of 1×PBS-0.05% Tween.
6. With a 200 μL multichannel pipette, 50 μL of each Fab dilution was transferred from the dilution plate to the coated plate.
7. The plate was sealed and incubated for 30 minutes at room temperature while shaking at 450 rpm on a platform shaker.
8. After 30 minutes, 50 μL of the biotinylated clone 7B07 (hFab) at 400 ng/ml was added to each well to reach a final concentration of 200 ng/mL.
9. The plate was sealed and incubated for 1 hour at room temperature while shaking at 450 rpm on a platform shaker.
10. After this incubation, the plate was washed 5 times with 300 μL per well of 1×PBS-0.05% Tween.
11. Then detection was performed with 100 μL of Peroxidase Streptavidin (diluted at 1/5000 in 1×PBS-0.1% Casein).
12. The plate was sealed and incubated for 1 hour at room temperature while shaking at 450 rpm on a platform shaker.
13. 15 minutes before the end of the incubation time, the TMB was transferred at room temperature.
14. After this incubation time, the plate was washed 5 times with 300 μL of 1×PBS-0.05% Tween per well.
15. Finally, 100 μL of TMB was added per well. The reaction was performed during 10 minutes while shaking at 450 rpm, before being stopped by the addition of 100 μL per well of 0.5 M H₂SO₄.
16. Absorbance was measured at 450 nm (reference at 620 nm) with a Tecan instrument and Magellan software software version n° 7.2.

### Binding Screening of the Fab Periplasmic Extract Via OctetRed96

The BLI is a label-free technology for measuring biomolecular interactions. It is an optical analytical technique that analyzes the interference pattern of white light reflected from two surfaces: a layer of immobilized protein on the biosensor tip, and an internal reference layer. Any change in the number of molecules bound to the biosensor tip causes a shift in the interference pattern that can be measured in real-time.

The BLI Octet screening assay was used to test the binding capacity of the Fabs produced as periplasmic extract after the selection step.
1. Human Gal10-His tagged was diluted in Kinetic Buffer (200 μg/mL) and captured on Anti-Penta His 1K tips until a capturing level of 1 nm was reached (Loading step, 30 seconds).
2. Periplasmic extracts were diluted 1:5 in Kinetic buffer and applied for 120 seconds (association) followed by 120 seconds of dissociation.
3. Regeneration of the tips was done by 2 washings steps of 10 seconds in Glycine pH 1.5.
4. To assess the non-specific binding of the periplasmic extract to the sensor tips, reference Anti-Penta His-1K tips were used. These tips were coated with an irrelevant His tagged protein (a-CD70 107B8-sortase-His D3 control) to a level of 1 nm and were incubated with the periplasmic extracts.
5. BLI binding analysis for Gal10-His was performed at 25° C. using the OctetRed 96 (ForteBio).
6. A Double Reference analysis was performed, the binding on the reference tips and the Buffer (on Gal10-His coated tips) was removed from the signal detected with the coated tips.
7. Kinetic parameters were determined by fitting the binding of the Fabs on captured Gal10-His with a 1:1 binding model (Align Y-axis to baseline, Inter-step correction to dissociation, Savitzky-Golay Filtering, Curve fitting Local/Partial) using the ForteBio Data analysis 9.0 software. Note that 1 column of Anti-Penta His-1K was used per PMP and was discarded after 10 regeneration cycles.

### Binding Characterization of the Molecules Anti-Gal10 on Biacore 3000

Coating of CM5 Chip with Gal10-his

In order to determine the binding properties of the periplasmic extracts, a CM5 sensor chip was coated with 2500-3000 RU of Gal10-His.
1. The coating material was buffered in Acetate pH 5.5 at a concentration of 20 μg/mL.
2. The Gal10-His was then immobilized on the NHS esters activated at the surface of the CM5 chip by reaction with EDC and NHS.
3. After immobilization of the target, washing and deactivation of the free NHS esters was performed with ethanolamine.
   Coating of CM5 Chip with Anti-His Tag Antibody

To determine the binding capacity of the anti-Gal10 constructs on Biacore 3000, a capture approach was used. For this purpose the BioRad monoclonal Mouse Anti-Histidine Tag, Clone AD1.1.10, was coated via amine coupling on the 4 channels of a CM5 chip with a final response of 4000 RU.
1. The coating material was buffered in Acetate pH 4.5 at a concentration of 20 μg/mL.
2. The monoclonal mAbs was then immobilized on the NHS esters activated at the surface of the CM5 chip by reaction with EDC and NHS.
3. After immobilization of the target, washing and deactivation of the free NHS esters was performed with Ethanolamine.
   Screening of Binding Properties of the scFv Periplasmic Extracts (Off-Rate Screening) For the screening of the binding properties of the periplasmic extracts, a chip coated with 2600 RU Gal10-His was used.
1. The peris stored in a 96 well plate at −20° C. were thawed and diluted 1:5 by adding 10 μL of peri to 40 μL of HBS-EP running buffer in a new round bottom 96 well Biacore plate. The plate was sealed.
2. After a cycle of priming, normalization, and priming, the coated CM5 chip was equilibrated to the running buffer with 4 cycles of 10 μL HBS-EP buffer pH 7.4.
3. Using a flow rate of 5 L/minute, 25 μL of diluted periplasmic extracts was injected.
4. After 15 minutes, regeneration of the chip was performed by injecting 6 μL of 8 mM NaOH (Quickinject at flowrate 100 μL/min) before to inject a new sample.
5. Data analysis was done using the “BIAevaluation” software version n° 4.1.1 with 2-1 and 4-3 subtraction. The sensorgram buffer only was used as a reference, and the signal was removed (Y-transformed). Finally, a fitting model was used to determine the affinity and the off-rate of the clone for Gal10-His (Fit Kinetics separate ka/kd/Dissociation/Langmuir dissociation).

### Screening of Binding Properties of the Fabs (Off-Rate Screening)

For the screening of the binding properties of the periplasmic extracts and purified anti-Gal10 molecules, a chip coated with 4000 RU anti-His tag antibody was used.
1. The purified Fabs were diluted to multiple concentrations. Calibrators and QC samples (70%, 100%, and 130% of the test concentration) were included.
2. After a cycle of priming, normalization, and priming, the coated CM5 chip was equilibrated to the running buffer with 2 cycles of 60 μL HBS-EP buffer pH 7.4.
3. As a first step, the Gal10-His was captured on 1 channel of the coated CM5 chip via the injection of 20 μL (“Quickinject”, the flow was set to 30 μL/minute) of the diluted Gal10-His until a final capturing level around 500 RU was reached.
4. As a second step, one dilution of the clones in the human Fab backbone was injected on the captured Gal10-His (2 channels, 60 μL, “Kinject”).
5. After a dissociation time of 100 seconds, regeneration of the chip was done by one injection of 8 μL of 10 mM Glycine-HCl pH 2.4 (“Quickinject”).
6. Data analysis was done using the “BIAevaluation” software version n° 4.1.1 with Fc4-Fc3 or Fc2-Fc1 subtraction. Normalization of the sensorgram was done after the capturing level of the Gal10-His (around 200 seconds). The sensorgram buffer only was used as a reference, and the signal was removed (Y-transform/Curve-Curve 2 (Blank Run subtraction)). Finally, a fitting model with mass transport effect was used to determine the affinity and the off-rate of the clone for Gal10-His (Fit Kinetics simultaneous ka/kd/Binding with mass transfer/Local Rmax).

### Human CLC Dissolution (Time Lapse)

1. The outer wells of the 96-well plate were filled with 300 μL PBS buffer.
2. Sufficient vacuum grease was applied at the edges of the plate to avoid evaporation.
3. 2 μL drops of diluted CLC solution (0.7 mg/mL in PBS) were spotted in the 96-well plate.
4. Next, the transparent plastic cover lid was placed on top of the plate.
5. The plate was then placed in a 96-well plate holder of the spinning disk confocal microscope and two positions per well were determined.
6. After all positions have been chosen, the plate was removed from the holder, the lid was removed, and 2 μL of antibody solution was added to the 2 μL drop of CLC in the well. As a control condition, 2 μL of PBS buffer was added.
7. Each Fab condition was imaged multiple times to ensure reproducibility.
8. The plate was then sealed and placed back in the holder of the microscope. Additionally, the plate was fixed with adhesive tape to further ensure no stage drift. Then, all positions were rechecked and refined. The positions were saved in the metadata output by the software.
9. Samples were then imaged every 3-5 minutes on an Axio Observer.Z1 (Zeiss) equipped with a CSU-X1 Yokogawa spinning-disk head (Yokogawa Corporation) and a Zeiss AxioCam Mrm (Zeiss), with a Plan-ApoChromat 10x dry objective (NA 0.30, DIC). Images were acquired continuously for up to 3 hours (an entire cycle takes roughly 72 seconds).
10. Image reconstruction and data analysis are performed with ImageJ (NIH). In short, sum projections are created for each stack, then an edge filter was applied to the images at which point a threshold can be applied. Measurements were made on each image to calculate the overall area taken by the crystals, and data was then exported to an Excel file where it was normalized to time point zero and plotted as a function of time. Data of replicate samples were merged for statistical analysis. The overall size of the CLC was measured over time and plotted on GraphPad Prism 7.01. The EC50 (50% dissolution) and EC10 (90% dissolution) values for each antibody, calculated with a non-linear regression (log (agonist) vs. response Variable slope (four parameters)).

### Human CLC Dissolution (Time Lapse)

1. The aim was to determine the activity of 14 antibody fragments in the 1536-well High Content assay in the presence of 1 μg Gal10 per well.
2. 3.8 mg/mL Gal10 was incubated overnight (16 h) at 20° C. in a 1.5 mL tube with 38 μg/mL TEV protease.
3. 4 mg/mL Gal10 was vortexed for 30 seconds and diluted to 0.2 mg/mL in PBS 30 minutes post-vortex.
4. Assay controls were added to the 1536-well plate: 10 points serial dilutions of 1D11 was prepared and 1 μL manually added (n=3) to the 1536-well plate. 1 μL of PBS and 1D11 (3 mg/mL) were added (n=64).
5. Antibodies were diluted to 1.5 mg/ml and 1 μL was transferred (n=4) to the 1536-well plate.
6. 5 μL of Gal10 (1 μg total) was dispensed using a bulk dispenser (BioTek MultiFlo) onto the antibody in the plate.
7. The plate was imaged (InCell 2200) 2, 5, 7, and 16 hours after antibody addition.

The images were segmented using an algorithm developed in-house to detect individual crystals and calculate the total crystal area per well.

### Example 5: Further Temperature Stress Stability Study Results

All clones were stored at 2-8° C. for up to 48h and the protein concentration of the sample was adjusted to 10 mg/mL in the original formulation buffer under aseptic conditions.

The clones were then stored under different temperature conditions (+5° C., +25° C. and +37° C.) for 4 weeks and tested for stability in weekly intervals. Stability was tested after several freeze thaw cycles (1x, 5x and 10x cycles) and after thermal stress (thermotolerance) within a range of denaturing temperatures spanning the range of 55° C. to 80° C.

### 2.1 Temperature Stress Stability Study Results

### Appearance and Visual Inspection

Two individuals conducted a visual inspection of the stability of the samples at all time-points and all temperature stress conditions (0.5 mL fill in 1.5 mL in transparent, glass vials). Overall, the samples had the same visual appearance (Table 31).

TABLE 31
Overview of visual inspection results of the clones upon temperature stress test.

| | Study | Storage at +5° | | | Storage at +25° | | | Storage at +37° | | |
|---|---|---|---|---|---|---|---|---|---|---|
| | Start | C. for 4 W | | | C. for 4 W | | | C. for 4 W | | |
| Clone | T0 | T1 W | T2 W | T4 W | T1 W | T2 W | T4 W | T1 W | T2 W | T4 W |
| 18C06 | A/A | A/A | AF/A | A/AF | B/A | B/A | A/B | B/B | B/A | A/B |
| 20H09 | A/A | A/A | AF/AF | A/AF | A/A | A/A | A/A | B/B | B/A | A/B |
| 23H09 | A/A | A/A | B/A | A/B | A/A | AF/A | A/B | A/B | B/A | A/B |
| 24F02_N53A | A/A | A/A | A/A | A/A | A/A | A/A | B/AF | A/A | A/A | B/A |

A: no visible particles;
B: few visible particles (<5);
F: fibers

### Turbidity

For a qualitative detection of scattering and aggregation, particles were evaluated by absorption spectra acquisition on a spectrophotometer at two wavelengths. The results were expressed as an aggregation index (A.I.) at 340 nm and 500 nm for all samples. A.I. was determined according to the following formulae: A.I._{340 nm}=A_{340 nm}/(A_{280 nm}−A_{340 nm}) and A.I._{500 mm}=A_{500 mm}/(A_{280 nm}−A_{500 nm}).

For each sample, the corresponding buffer, a negative control (sterile, filtered mQ water) and a positive control (a protein sample known to contain elevated aggregates) were also subjected to the same spectrophotometric analyses. No distinct differences were observed between the clones in terms of the aggregation indexes as compared to the formulation buffer or amongst clones for any of the temperature-stressed conditions applied (Table 32).

TABLE 32
Turbidity and aggregation indexes A.I. 340 and A.I. 500 nm obtained for clones
throughout the study for the different temperature stressed conditions.

| | Storage at +5° | | | Storage at +25° | | | Storage at +37° | | |
|---|---|---|---|---|---|---|---|---|---|
| | C. for 4 W | | | C. for 4 W | | | C. for 4 W | | |
| | T1 W | T2 W | T4 W | T1 W | T2 W | T4 W | T1 W | T2 W | T4 W |
| A.I. 340 nm | | | | | | | | | |
| Formulation buffer | 0.03 | 0.03 | 0.03 | 0.03 | 0.03 | 0.03 | 0.03 | 0.03 | 0.03 |
| 18C06 | 0.03 | 0.03 | 0.03 | 0.03 | 0.03 | 0.03 | 0.03 | 0.03 | 0.03 |
| 20H09 | 0.03 | 0.03 | 0.03 | 0.03 | 0.03 | 0.03 | 0.03 | 0.03 | 0.03 |
| 23H09 | 0.03 | 0.03 | 0.03 | 0.03 | 0.03 | 0.03 | 0.03 | 0.03 | 0.03 |
| Positive Control | 0.07 | 0.09 | 0.10 | ND | ND | ND | ND | ND | ND |
| Negative control | 0.03 | 0.03 | 0.03 | ND | ND | ND | ND | ND | ND |
| Formulation buffer | 0.04 | 0.04 | 0.04 | 0.04 | 0.04 | 0.04 | 0.04 | 0.04 | 0.04 |
| 24F02_N53A | 0.04 | 0.04 | 0.04 | 0.04 | 0.04 | 0.04 | 0.04 | 0.03 | 0.03 |
| Positive Control | 0.09 | 0.09 | 0.09 | ND | ND | ND | ND | ND | ND |
| Negative control | 0.04 | 0.04 | 0.04 | ND | ND | ND | ND | ND | ND |
| A.I. 500 nm | | | | | | | | | |
| Formulation buffer | 0.02 | 0.02 | 0.02 | 0.02 | 0.02 | 0.02 | 0.02 | 0.02 | 0.02 |
| 18C06 | 0.01 | 0.01 | 0.01 | 0.01 | 0.01 | 0.01 | 0.01 | 0.01 | 0.01 |
| 20H09 | 0.01 | 0.01 | 0.02 | 0.01 | 0.01 | 0.01 | 0.01 | 0.02 | 0.01 |
| 23H09 | 0.02 | 0.01 | 0.01 | 0.01 | 0.02 | 0.01 | 0.01 | 0.01 | 0.01 |
| Positive Control | 0.04 | 0.04 | 0.05 | ND | ND | ND | ND | ND | ND |
| Negative control | 0.01 | 0.01 | 0.01 | ND | ND | ND | ND | ND | ND |
| Formulation buffer | 0.02 | 0.02 | 0.02 | 0.02 | 0.02 | 0.02 | 0.02 | 0.02 | 0.02 |
| 24F02_N53A | 0.01 | 0.01 | 0.01 | 0.01 | 0.01 | 0.01 | 0.01 | 0.01 | 0.01 |
| Positive Control | 0.04 | 0.04 | 0.04 | ND | ND | ND | ND | ND | ND |
| Negative control | 0.01 | 0.01 | 0.01 | ND | ND | ND | ND | ND | ND |

ND = not determined.

### Submicron Particle Detection by Dynamic Light Scattering (DLS)

DLS analysis was carried out for all clones and formulation samples at the 4-week time point of the study (T4W) for all storage conditions. Measurements took place in triplicate preparations with a DynaPro Nanostart instrument. Control samples without any stress applied, samples kept for 4W under different stressed conditions (at TO and at T4W) were analyzed side-by-side. Percent mass, hydrodynamic radius of molecule, percent polydispersity (% PD) and polydispersion index (PDI) were used to monitor the distribution profiles of the submicron particles in solution.

The formulation buffers were not suitable for any of the storage conditions applied, before or after filtration (0.2 μm). Aliquots stored for 4W (T4W) at +25° C. and +37° C. were not suitable for any of the candidates. Additionally, solutions for clones 20H09 and 23H09 were not suitable for the non-stressed samples at the study start (TO). All samples for all 4 candidates were thus filtered and re-analyzed (triplicate measurements). DLS profiles at T4W for the samples were relatively similar and had several peaks in intensity but the non-monomeric species were in general negligible as the percent mass numbers indicate (Table 33). The radius of the main peak for all candidates was in agreement with the expected size of the proteins. The polydispersity of a sample is an indication of homogeneity. Whilst polydispersity was variable between the replicates for each of the clones, overall the polydispersity scores indicated that there was a narrow size distribution of particles in all of the samples. The majority of post-filtration samples at different storage conditions tested were found to be multimodal, which indicates the presence of particles with varying sizes in the samples. Samples from clone 24F02-N53A were least prone to multimodal scattering indicating that these samples had a more narrow particle size distribution.

TABLE 33
Summary of DLS data

| | | Range of | | | | | | | Number | Number of |
|---|---|---|---|---|---|---|---|---|---|---|
| | Radius | radius | Mass | Range of | PD | Range of | | Range | multimodal | unsuitable |
| Samples | (nm) | (nm) | (%) | mass (%) | (%) | PD (%) | PDI | PDI | samples | samples |
| g18C06 T0 | 3.3 | 3.2-3.5 | 100 | 100 | 11.8 | 11.0-12.7 | 0.12 | 0.11-0.13 | 1/3 | 0/3 |
| g18C06 T4 | 3.1 | 3.0-3.2 | 100 | 100 | 11.8 | 6.6-19.5 | 0.12 | 0.07-0.19 | 0/3 | 0/3 |
| W + 5° C. | | | | | | | | | | |
| g18C06 T4 | 3.2 | 3.2-3.3 | 100 | 100 | 12.0 | 7.1-15.7 | 0.12 | 0.07-0.16 | 2/3 | 0/3 |
| W + 25° C. | | | | | | | | | | |
| g18C06 T4 | 3.2 | 3.1-3.2 | 100 | 100 | 9.1 | 5.7-11.6 | 0.09 | 0.06-0.12 | 3/3 | 0/3 |
| W + 37° C. | | | | | | | | | | |
| g20H09 T0 | 3.7 | 3.6-3.9 | 99.9 | 99.9-100 | 13.2 | 11.3-14.6 | 0.13 | 0.11-0.15 | 3/3 | 0/3 |
| g20H09 T4 | 3.5 | 3.4-3.5 | 100 | 100 | 12.4 | 11.8-13.4 | 0.12 | 0.12-0.13 | 0/3 | 0/3 |
| W + 5° C. | | | | | | | | | | |
| g20H09 T4 | 3.5 | 3.5 | 100 | 100 | 12.6 | 11.9-14.0 | 0.13 | 0.12-0.14 | 3/3 | 0/3 |
| W + 25° C. | | | | | | | | | | |
| g20H09 T4 | 3.8 | 3.7-3.9 | 99.9 | 99.9 | 21.4 | 17.5-23.5 | 0.21 | 0.18-0.23 | 3/3 | 0/3 |
| W + 37° C. | | | | | | | | | | |
| g23H09 T0 | 3.5 | 3.4-3.7 | 100 | 100 | 11.3 | 10.4-11.9 | 0.11 | 0.10-0.12 | 3/3 | 0/3 |
| g23H09 T4 | 3.3 | 3.2-3.3 | 100 | 99.8-100 | 11.1 | 9.8-18.2 | 0.11 | 0.10-0.18 | 1/3 | 0/3 |
| W + 5° C. | | | | | | | | | | |
| g23H09 T4 | 3.6 | 3.6 | 100 | 99.9-100 | 14.1 | 11.5-15.5 | 0.14 | 0.12-0.15 | 3/3 | 0/3 |
| W + 25° C. | | | | | | | | | | |
| g23H09 T4 | 3.6 | 3.6 | 100 | 99.9-100 | 16.0 | 14.3-18.2 | 0.16 | 0.14-0.18 | 3/3 | 0/3 |
| W + 37° C. | | | | | | | | | | |
| g7B07_N53A T0 | 3.4 | 3.3-3.5 | 100 | 100 | 11.9 | 11.8-12 | 0.12 | 0.12 | 0/3 | 0/3 |
| g7B07_N53A | 3.3 | 3.2-3.4 | 100 | 100 | 11.1 | 10.6-11.6 | 0.11 | 0.11-0.12 | 0/3 | 0/3 |
| T4 W + 5° C. | | | | | | | | | | |
| g7B07_N53A | 3.3 | 3.2-3.3 | 100 | 100 | 11.1 | 10.1-13.2 | 0.11 | 0.10-0.13 | 3/3 | 0/3 |
| T4 W + 25° C. | | | | | | | | | | |
| g7B07_N53A | 3.2 | 3.2-3.3 | 100 | 100 | 10.9 | 9.6-13.2 | 0.11 | 0.10-0.13 | 3/3 | 0/3 |
| T4 W + 37° C. | | | | | | | | | | |
| g24F02_N53A T0 | 3.5 | 3.4-3.5 | 100 | 100 | 12.2 | 11.9-12.7 | 0.12 | 0.12-0.13 | 0/3 | 0/3 |
| g24F02_N53A | 3.5 | 3.5 | 100 | 100 | 16.4 | 11.9-20.0 | 0.16 | 0.12-0.20 | 0/3 | 0/3 |
| T4 W + 5° C. | | | | | | | | | | |
| g24F02_N53A | 3.5 | 3.5 | 100 | 100 | 17.7 | 14.6-19.5 | 0.18 | 0.15-0.19 | 0/3 | 0/3 |
| T4 W + 25° C. | | | | | | | | | | |
| g24F02_N53A | 3.6 | 3.5-3.6 | 100 | 100 | 16.6 | 12.1-19.6 | 0.17 | 0.12-0.20 | 0/3 | 0/3 |
| T4 W + 37° C. | | | | | | | | | | |
| Buffer T4 | Not suitable | | | | | | | | | 3/3 |
| W + 5° C. | | | | | | | | | | |
| Buffer T4 | Not suitable | | | | | | | | | 3/3 |
| W + 25° C. | | | | | | | | | | |
| Buffer T4 | Not suitable | | | | | | | | | 3/3 |
| W + 37° C. | | | | | | | | | | |

### Protein Concentration

Protein concentration was evaluated at A280 nm (Nanodrop) for temperature-stressed samples as well as the freeze-thaw stressed samples (FIG. 3). Broadly, no protein losses were observed throughout for any of the clones tested at any of the storage temperatures.

For clone 18C06 stored at +37° C. for 4W, the result was atypical. However, analysis of an independent aliquot, which was retained as a reserve sample (under the same experimental conditions), was nevertheless measured within the expected range.

### Binding Activity by SPR

The functional active concentration of the candidates was then assessed by SPR on a Biacore 3000 instrument (FIG. 4). All samples were evaluated with methods meeting the standard qualification criteria. Aliquots were tested at predefined test concentrations against titration curves from reference samples (T0, −80° C. storage) and in the presence of quality control (QC) samples covering a range of 70-130% relative activity (% RA) towards the reference material.

Almost all temperature-stressed samples from the four candidate clones retained more than 90% relative activity regardless of the conditions at which they were stored (FIG. 4). Similar observations were made with samples subjected to freeze-thawing cycles and low pH (FIG. 4). The only atypical result recorded was for clone 18C06 stored for 4W at +37° C., although testing an independent aliquot of the same sample that was handled under the same conditions was observed to retain more than 90% RA (FIG. 4).

### Size Purity by SE-HPLC

Purity by SE-HPLC was assessed with an Agilent 1260 Infinity II chromatographic system, equipped with a quaternary pump, automatic injector, on-line degasser and a DAD detector, column thermo-stated compartment (21° C.), and auto-sampler set at 6° C. The detector was set to wavelengths of 280 nm and 214 nm simultaneously to monitor size variants. Samples were analyzed from all storage conditions and at all time-points.

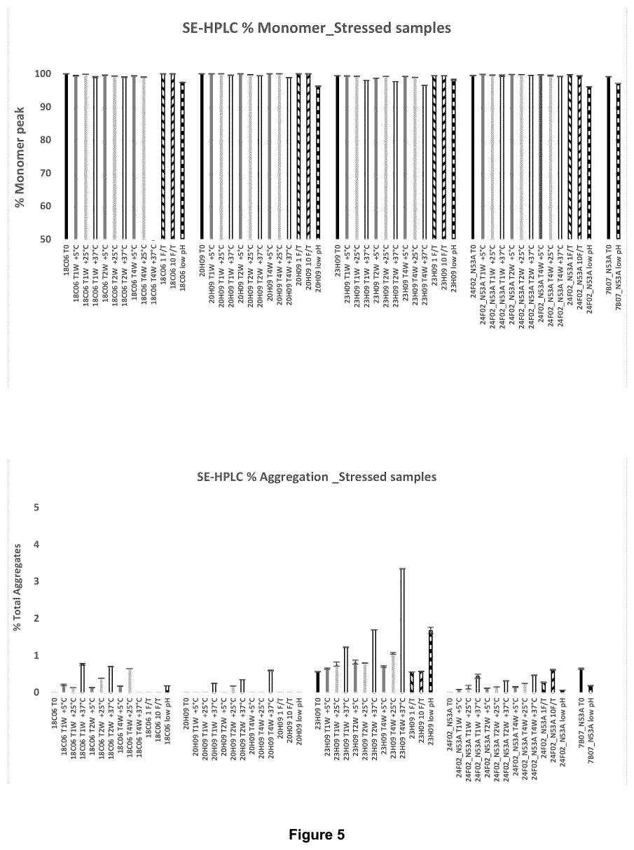

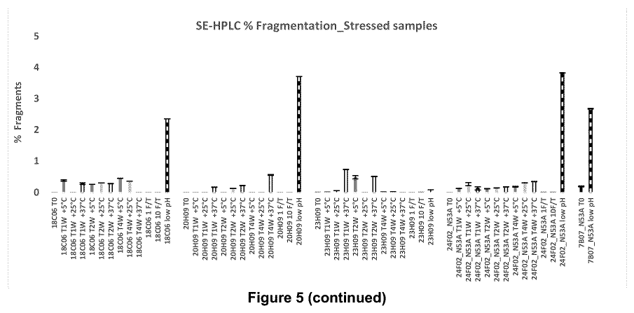

All temperature-stressed, freeze-thawed and low pH samples from the four clones were observed to have greater than 95% purity (FIG. 5). The percentage aggregation and fragmentation remained below 1% for the majority of temperature-stressed samples (FIG. 5). The only exception was clone 23H09 samples, which exhibited a temperature-induced increase in aggregate formation (FIG. 5).

Samples subjected to up to 10 freeze-thaw cycles, exhibited a purity comparable to the reference material for all clones (FIG. 5). For samples that underwent a low pH stressor, minor aggregation was detected for clone 23H09 samples (FIG. 5). All other clones tested demonstrated some fragmentation after application of low pH stress (FIG. 5). However, in all cases, the percentage of impurities remained below 5% (FIG. 5).

Purity by Capillary Gel Electrophoresis (cGE)

Purity by cGE was assessed using a lab-on-a-chip analysis using an Expert 2100 Bioanalyzer device (Agilent). Samples were analyzed under reducing and non-reducing conditions at the final time-point of each study.

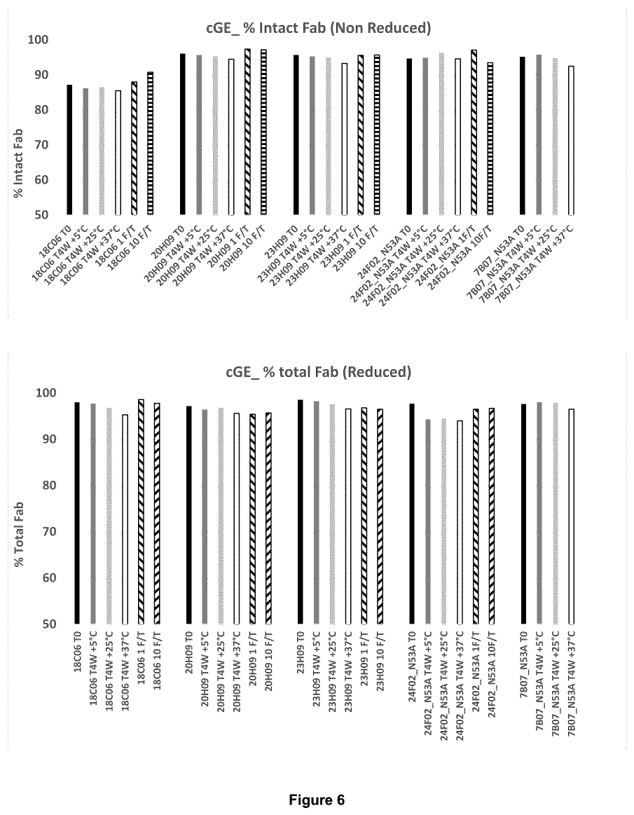

Different storage conditions had no impact on the purity of the clones, except for clone 18C06 (FIG. 6). Samples containing this clone did not demonstrate purity of the main peak of more than 90% under non-reducing conditions (FIG. 6). This was also the case for the unstressed reference sample containing clone 18C06 (FIG. 6). For the rest of the samples containing the other three clones, purity exceeded 90% under non-reducing conditions irrespective of the storage temperature, time-point of analysis or the number of freeze thaw cycles they were subjected to (up to 10) (FIG. 6). Under reducing conditions, all samples tested demonstrated greater than 95% purity (FIG. 6).

### Thermotolerance

To evaluate the thermal stability of the candidates, the clones underwent a gradient temperature decomposition test spanning the temperature range of 55-85° C. After application of this stress, in a Biometra Thermocycler programmed to apply several denaturing temperatures, the binding activity of the stressed samples was assessed on Biacore 3000 to identify the temperature at which 50% binding activity towards the Gal10 was abolished. A 100% binding activity was attributed to a non-stressed sample of each clone which was then analyzed side-by-side by SPR. Samples for all clones demonstrated a 50% activity loss at elevated temperatures (Table 34). The most stable clone identified according to this analytical approach was candidate 24F02_N53A which, along with the reference clone 7B07_N53A, illustrated melting temperatures above 70° C.

TABLE 34
Overview of thermal stability of the lead candidates

| Decomposition | | 50% binding |
|---|---|---|
| Temperature | | activity loss by |
| range | Clone | SPR (° C.) |
| 55-75° C. | 18C06 | 65.4 |
| 55-75° C. | 20H09 | 66.3 |
| 55-75° C. | 23H09 | 67.5 |
| 65-85° C. | 24F02_N53A | 74.7 |
| 58-80° C. | 7B07_N53A | 72.9 |

### Summary Tables

A tabulated summary illustrating several core characteristics of the selected Fabs (before any type of stress was applied) is provided in Table 35. The 7B07_N53A Fab is included for comparison and reference purposes.

A tabulated summary of attributes of the selected Fabs under different conditions is provided in Table 36. A tabulated summary illustrating several physicochemical characteristics of the individual candidates after freeze thaw, low pH and thermal stress is provided in Tables 37-40.

TABLE 35
Overview of lead Fab candidates' attributes

| | Criteria | | | | | |
|---|---|---|---|---|---|---|
| Attribute | (Target) | g18C06 | g20H09 | g23H09 | g24F02_N53A | (g7B07_N53A) * |
| Format | Fab | Fab | Fab | Fab | Fab | Fab |
| Human | % identity to | 100% | 98.8% | 98.8% | 100% | 98.8% |
| identity | human germ | | | | | |
| | V-region | | | | | |
| Immunogenicity | Epibase | Human | Human | Human | Human | NT |
| | score: human | Low | Low | Low | Low | |
| | Immunogenicity | | | | | |
| | score: low (L) | | | | | |
| Cross- | Cyno cross- | No | No | Yes | Yes | No |
| reactivity | reactivity | | | | | |
| Binding | KD (affinity) | Yes | No | Yes | Yes | No |
| activity | ≤15 nM | (1.3 nM) | (17.7 nM) | (5.3 nM) | (1.4 nM) | (145.0 nM) |
| | kd | Yes | No | Yes | Yes | No |
| | (off rate) | (4.9E−04/s) | (4.7E−02/s) | (3.1E−03/s) | (1.8E−03/s) | (79.0E−03/s) |
| | ≤5E−03/s | | | | | |
| pI | Report result | 9.55 | 8.32 | 7.47 | 8.03 | NT |

NT: not tested;
L: low immunogenicity score

TABLE 36
Overview of attributes of the four Fab clones after several stresses: binding activity, biological activity on CLCs, purity
by CE-SDS (reduced and non-reduced) and SE-HPLC, post translational modifications and particles evaluation by DLS and FCM

| Attribute | Criteria (Target) | g18C06 | g20H09 | g23H09 | g24F02_N53A | (g7B07_N53A)* |
|---|---|---|---|---|---|---|
| Binding activity | Stressed ⁽¹⁾/Nebulized ⁽²⁾ | No (?)/No | No/No | No/No | No/No | No/No |
| | (<20% loss in relative | 82% (93%) ³/101% | 101%/97% | 93%/119% | 103%/111% | 95%/94% |
| | activity by SPR) | | | | | |
| Biological | Unstressed material (T0) | Assay 1: 281 | Assay 1: 260 | Assay 1: 252 | Assay 1: NA | Assay 1: 309 |
| activity | Stressed ⁽¹⁾/Nebulized ⁽²⁾ | Assay 1: 232/485 | Assay 1: 272/395 | Assay 2: 190 | Assay 2: 191 | Assay 1: 379/584 |
| (recGal10 | Report result EC50 | Assay 2: NA | Assay 2: NA | Assay 1: 361/297 | Assay 1: NA | Assay 2: NA |
| crystal | (μg/mL; T2 h) | | | Assay 2: 191/209 | Assay 2: 164/159 | |
| dissolution) | Stressed ⁽¹⁾/Nebulized ⁽²⁾ | No/Yes | No/Yes | No/No (Assay 2) | No/No | No/Yes |
| | % Relative Activity | 121%/58% | 96%/66% | 100%/91% | 117%/120% | 82%/53% |
| | towards the reference T0 | | | | | |
| | after 2 h incubation | | | | | |
| | (<20% loss in relative | | | | | |
| | activity) | | | | | |
| Purity (SE- | Stressed/Nebulized, | SE-HPLC: Yes | Yes | Yes | Yes | Yes |
| HPLC/CE-SDS) | all conditions | CE-SDS: ~87% | | | | |
| | (≥90% main, ≤10% | monomer | | | | |
| | HMW & LMW) | | | | | |
| | Stressed/Nebulized, | Yes | Yes | Yes | Yes | Yes |
| | all conditions | | | (T-induced: | | |
| | (ΔT0 ≤ 5%, | | | 3.3% HMW sp; | | |
| | ΔHMW < 2% post- | | | low pH: 1.7% | | |
| | neb) | | | HMW sp) | | |
| Post- | Major liabilities T- | minor; | minor | minor | minor | minor |
| Translational | stressed samples, | hotspot in LC | | | | |
| Modifications | all conditions | | | | | |
| | Major liabilities | No | No | No | No/(minor in | No |
| | nebulized all | | | | LC_CDRs) | |
| | conditions/low pH | | | | | |
| Subvisible | Stressed, all | Non-monomeric | Non-monomeric | Non-monomeric | Non-monomeric | Non-monomeric |
| particles | conditions (T4 W) | species | species | species | species | species |
| (DLS after | | negligible | negligible | negligible | negligible | negligible |
| filtration) | | | (<0.1%) ⁽⁴⁾ | (<0.2%) ⁽⁴⁾ | | |
| | Nebulized (T0 and | Non-monomeric | Not suitable | Not suitable | Non-monomeric | Non-monomeric |
| | T4 W) | species | PDI: 0.13/NA | PDI: 0.16/NA | species | species |
| | PDI range (T4 W) | negligible | | | negligible | negligible |
| | pre-/post-neb | (<0.1%) | | | (<0.1%) | PDI: 0.12/ |
| | | PDI: 0.13/ | | | PDI: 0.16/ | 0.11-0.16 |
| | | 0.06-0.13 | | | 0.12-0.14 | |
| Subvisible | Total counts/mL | 6.547/16.201 | 1.476/212.723 | 302/241.257 | 2.456/21.550 | NA/NA |
| particles | (<30.000 | 266/23.363 | 14.063/291.462 | 854/292.352 | 2.606/19.713 | 1.827/12.104 |
| (FCM) | counts/mL) | | | | | |
| | T0 (pre/post | | | | | |
| | nebulization) | | | | | |
| | T4 W (pre/post | | | | | |
| | nebulization) | | | | | |
| | >2 μm/mL (<10.000 | 559/2.204 | 292/28.532 | 139/31.724 | 642/4.732 | NA/NA |
| | counts/mL) | 137/3.992 | 1.576/49.344 | 397/32.911 | 442/4.328 | 802/3.118 |
| | T0 (pre/post | | | | | |
| | nebulization) | | | | | |
| | T4 W (pre/post | | | | | |
| | nebulization) | | | | | |

comparison/reference
⁽¹⁾ samples correspond to aliquots kept at +37° C. for 4 weeks
⁽²⁾ samples correspond to aliquots kept at +5° C. for 4 weeks prior to nebulization. In brief, samples were nebulized with an Aerogen Solo active vibrating mesh nebulizer that converted a drug solution into an inhalable aerosol. Three different devices for each time-point were used per clone and results are reported by the device (nebulizer) serial number. In case of device fouling, indicated by longer nebulization times
For comparison/reference
⁽¹⁾ samples correspond to aliquots kept at +37° C. for 4 weeks
⁽²⁾ samples correspond to aliquots kept at +5° C. for 4 weeks prior to nebulization. In brief, samples were nebulized with an Aerogen Solo active vibrating mesh nebulizer that converted a drug solution into an inhalable aerosol. Three different devices for each time-point were used per clone and results are reported by the device (nebulizer) serial number. In case of device fouling, indicated by longer nebulization times than usual for a given size of aliquot, spare devices of the same type were available to complete the nebulization with triplicate devices.
³ % RA for this sample after 4 weeks storage at +37° C. was measured at 82% (Eppendorf) and at 93% (glass vial) for two independent samples
⁽⁴⁾ samples for 20H09 and 23H09 were not suitable without filtration even for the non-stressed aliquots; information in this row illustrates results after filtration for all clones all types of samples

TABLE 37
Overview of several physicochemical characteristics for Fab clone 18C06.

| | | | | Low pH Stress Test ⁽¹⁾ | | Thermotolerance |
|---|---|---|---|---|---|---|
| | | | | Before | After | 50% relative |
| | Freeze/Thaw stability | | | low pH | low pH | activity reached |
| Method | 1 F/T | (5 F/T) ⁽²⁾ | 10 F/T | stress | stress | by Biacore (° C.) |
| Protein | 10.3 | NT | 10.1 | NA | NA | NA |
| Concentration | | | | | | |
| (mg/mL) | | | | | | |
| pH value | | | | 7.43 | 3.70 | |
| Potency by | 106% | NT | 109% | 91% | 92% | 65.4 |
| Biacore | | | | | | |
| (% Relative | | | | | | |
| activity towards | | | | | | |
| reference) | | | | | | |
| CE-SDS | 98.6% | NT | 97.8% | NA | NA | NA |
| reducing | | | | | | |
| Report % | | | | | | |
| total Fab | | | | | | |
| CE-SDS non- | 88.0% | NT | 90.8% | NA | NA | NA |
| reducing | | | | | | |
| % main peak | | | | | | |
| SE-HPLC size | 100% | NT | 100% | 100% | 97.5% | NA |
| variants | ND | NT | ND | ND | 0.2% | NA |
| % monomer | ND | NT | ND | ND | 2.4% | NA |
| % aggregates | | | | | | |
| % degradation | | | | | | |

⁽¹⁾ the low pH stress test was achieved by reaching pH 3.7 with 0.1M glycine pH 3.0 and after a 2-h hold step at ambient conditions, the aliquot was brought back to neutralization with 1M Tris pH 8.0.
⁽²⁾ One aliquot has undergone 5 freeze thaw cycles but as per protocol, it was not analyzed as no atypical result was obtained for the 10 freeze thaw aliquot.
NT: not tested;
NA: not applicable;
ND: not detected

TABLE 38
Overview of several physicochemical characteristics for Fab clone 20H09.

| | | | | Low pH Stress Test ⁽¹⁾ | | Thermotolerance |
|---|---|---|---|---|---|---|
| | | | | Before | After | 50% relative |
| | Freeze/Thaw stability | | | low pH | low pH | activity reached |
| Method | 1 F/T | (5 F/T) ⁽²⁾ | 10 F/T | stress | stress | by Biacore (° C.) |
| Protein | 11.0 | NT | 10.7 | NA | NA | NA |
| Concentration | | | | | | |
| (mg/mL) | | | | | | |
| pH value | | | | 7.49 | 3.73 | |
| Potency by | 104% | NT | 110% | 105% | 103% | 66.3 |
| Biacore | | | | | | |
| (% Relative | | | | | | |
| activity towards | | | | | | |
| reference) | | | | | | |
| CE-SDS | 95.4% | NT | 95.7% | NA | NA | NA |
| reducing | | | | | | |
| Report % | | | | | | |
| total Fab | | | | | | |
| CE-SDS non- | 97.4% | NT | 97.2% | NA | NA | NA |
| reducing | | | | | | |
| % main peak | | | | | | |
| SE-HPLC size | 100% | NT | 100% | 100% | 96.3% | NA |
| variants | ND | NT | ND | ND | ND | NA |
| % monomer | ND | NT | ND | ND | 3.7% | NA |
| % aggregates | | | | | | |
| % degradation | | | | | | |

⁽¹⁾ the low pH stress test was achieved by reaching pH 3.7 with 0.1M glycine pH 3.0 and after a 2-h hold step at ambient conditions, the aliquot was brought back to neutralization with 1M Tris pH 8.0.
⁽²⁾ One aliquot has undergone 5 freeze thaw cycles but as per protocol, it was not analyzed as no atypical result was obtained for the 10 freeze thaw aliquot.
NT: not tested;
NA: not applicable;
ND: not detected

TABLE 39
Overview of several physicochemical characteristics for Fab clone 23H09.

| | | | | Low pH Stress Test ⁽¹⁾ | | Thermotolerance |
|---|---|---|---|---|---|---|
| | | | | Before | After | 50% relative |
| | Freeze/Thaw stability | | | low pH | low pH | activity reached |
| Method | 1 F/T | (5 F/T) ⁽²⁾ | 10 F/T | stress | stress | by Biacore (° C.) |
| Protein | 10.8 | NT | 10.6 | 5.7 | 2.9 | NA |
| Concentration | | | | | | |
| (mg/mL) | | | | | | |
| pH value | | | | 7.50 | 3.71 | |
| Potency by | 105% | NT | 89% | 107% | 112% | 67.5 |
| Biacore | | | | | | |
| (% Relative | | | | | | |
| activity towards | | | | | | |
| reference) | | | | | | |
| CE-SDS | 96.8% | NT | 96.5% | NA | NA | NA |
| reducing | | | | | | |
| Report % | | | | | | |
| total Fab | | | | | | |
| CE-SDS non- | 95.6% | NT | 95.7% | NA | NA | NA |
| reducing % | | | | | | |
| main peak | | | | | | |
| GP-HPLC size | 99.4% | NT | 99.4% | 99.4% | 98.3% | NA |
| variants | | | | | | |
| % monomer | 0.6% | NT | 0.6% | 0.6% | 1.7% | NA |
| % aggregates | ND | NT | ND | ND | ND | NA |
| % degradation | | | | | | |

⁽¹⁾ the low pH stress test was achieved by reaching pH 3.7 with 0.1M glycine pH 3.0 and after a 2-h hold step at ambient conditions, the aliquot was brought back to neutralization with 1M Tris pH 8.0.
⁽²⁾ One aliquot has undergone 5 freeze thaw cycles but as per protocol, it was not analyzed as no atypical result was obtained for the 10 freeze thaw aliquot.
NT: not tested;
NA: not applicable;
ND: not detected

TABLE 40
Overview of several physicochemical characteristics for Fab clone 24F02_N53A.

| | | | | Low pH Stress Test ⁽¹⁾ | | Thermotolerance |
|---|---|---|---|---|---|---|
| | | | | Before | After | 50% relative |
| | Freeze/Thaw stability | | | low pH | low pH | activity reached |
| Method | 1 F/T | (5 F/T) ⁽²⁾ | 10 F/T | stress | stress | by Biacore (° C.) |
| Protein | 9.6 | NT | 9.7 | NA | NA | NA |
| Concentration | | | | | | |
| (mg/mL) | | | | | | |
| pH value | | | | 7.43 | 3.46 | |
| Potency by | 105% | NT | 106% | 107% | 106% | 74.7 |
| Biacore | | | | | | |
| (% Relative | | | | | | |
| activity | | | | | | |
| towards | | | | | | |
| reference) | | | | | | |
| CE-SDS | 96.5% | NT | 96.7% | NA | NA | NA |
| reducing | | | | | | |
| Report % total | | | | | | |
| Fab | | | | | | |
| CE-SDS non- | 97.1% | NT | 93.5% | NA | NA | NA |
| reducing | | | | | | |
| % main peak | | | | | | |
| GP-HPLC size | 99.7% | NT | 99.4% | 99.8% | 96.1% | NA |
| variants | | | | | | |
| % monomer | 0.3% | NT | 0.6% | 0.2% | 0.1% | NA |
| % aggregates | ND | NT | ND | ND | 3.8% | NA |
| % degradation | | | | | | |

⁽¹⁾ the low pH stress test was achieved by reaching pH 3.7 with 0.1M glycine pH 3.0 and after a 2-h hold step at ambient conditions, the aliquot was brought back to neutralization with 1M Tris pH 8.0.
⁽²⁾ One aliquot has undergone 5 freeze thaw cycles but as per protocol, it was not analyzed as no atypical result was obtained for the 10 freeze thaw aliquot.
NT: not tested;
NA: not applicable;
ND: not detected

### Example 6: Post Translational Modification (PTM) Analysis

Structural characterization of the clones was performed at protein and peptide level using several analytical techniques (icIEF, online-desalting MS, RPLC-UV-MS on reduced protein, peptide map using RPLC-MS after tryptic digestion). The clones were analyzed for PTMs after they were subjected to several stress conditions including storage for 4W at different temperatures (+5° C., +25° C., +37° C.), nebulization before (TO) and after 4W storage at +5° C., and after a low pH hold step (2h/pH3.7). In all cases, the analyses were carried out side-by-side with the control unstressed reference material for each clone.

The findings can be summarized as:
The amino acid sequence of the four clones was confirmed at protein level, based on the molecular weight of each intact Fab (LC and VH+CH1) while the peptide sequence coverage was 100%.
The structural integrity before and after stress of the Fabs remained unchanged and was confirmed with intact protein and peptide mapping analyses which demonstrated and confirmed the presence of the (inter- and intra-chain) expected disulfide bridges. Only 18C06 (all samples including reference) was detected with a free cysteine in the LC instead of the expected bridge with the VH+CH1 part and instead, an alternative formation of a disulfide bridge between two closely located cysteines in the LC was detected.
N-terminal cyclization was detected for the common VH+CH1 part of the stressed clones which was clearly temperature-induced with net increase of the pyroglutamic acid after +37° C. storage (8.5-10.5%) and present but less pronounced after +25° C. storage (2.0-3.5%); this modification in the VH+CH1 was not identified for the low pH or nebulized samples. Additionally, for g18C06, the LC was found to be fully cyclic after nebulization.
Oxidation for all different stress conditions remained <1% for all clones.
Site-specific events such as isomerization and deamidation remained generally unaffected after the low pH stress step or nebulization for all clones.
For the temperature-stressed samples, clone 18C06 demonstrated a temperature-sensitive deamidation spot in one variable region of the LC chain (14.7% after 4 weeks at +37° C. Two moderate deamidation spots were detected (up to 5% each) at elevated temperature on a VH+CH1 peptide shared between all pre-lead candidates. For isomerization, one spot of the LC chain was detected with some limited increase in isomerization for g20H09, g23H09 and less for g24F02. A second peptide in LC, shared between constructs, was also associated with minor increase in isomerization (up to 1.3%).

TABLE 41
Principal post-translational modifications observed for the clones after 4weeks
storage at +37° C.

| | Sequence | | | | g24F02_ |
|---|---|---|---|---|---|
| Attribute | Location | g18C06 | g20H09 | g23H09 | N53A |
| EVQLLESGGGLVQPGG | VH + CH1 | 10.5% | 8.5% | 8.9% | 8.5% |
| SLR | (001-19) | pyro- | pyro- | pyro- | pyro- |
| (SEQ ID NO: 66) | | glutamate | glutamate | glutamate | glutamate |
| NTLYLQMNSLR | VH + CH1 | 4.6% | 2.5% | 4.0% | 4.4% |
| (SEQ ID NO: 67) | (072-087) | deamidation | deamidation | deamidation | deamidation |
| | | (TO: 1.8%) | (TO: 1.6%) | (TO: 1.6%) | (TO: 3.7%) |
| DYFPEPVTVSWNSGAL | VH + CH1 | 5.3% | 4.8% | 3.7% | 5.1% |
| TSG | (147-209) | deamidation | deamidation | deamidation | deamidation |
| VHTFPAVLQSSGLYSLS | | (T0: 1.0%) | (T0: 0.8%) | (T0: 1.2%) | (T0: 1.6%) |
| SVVT | | | | | |
| VPSSSLGTQTYICNVNH | | | | | |
| KPSNTK | | | | | |
| (SEQ ID NO: 68) | | | | | |
| ITCDGNNIGSK¹ | LC | | 3.7% | 2.5% | 1.9% |
| (SEQ ID NO: 69) | (020-030)² | | iso- | iso- | iso- |
| | | | merization | merization | merization |
| | | | (T0: 1.4%) | (T0: 1.3%) | (T0: 2.0%) |
| LLIYNANNR | LC | 14.7% | | | |
| (SEQ ID NO: 70) | (048-056) | deamidation | | | |
| | | (T0: 1.0%) | | | |
| AQAGDEADYYC | LC | | 1.2% | 0.6% | 1.8% |
| QVWDGSAAVFGGGTQ | (077-110) | | iso- | iso- | iso- |
| L | | | merization | merization | merization |
| TVLGQPK | | | (T0: 0.7%) | (T0: 0.5%) | (T0: 1.6%) |
| (SEQ ID NO: 71) | | | | | |
| ADSSPVK | LC | 1.2% | 1.2% | 1.4% | 1.3% |
| (SEQ ID NO: 72) | (150-161) | iso- | iso- | iso- | iso- |
| | | merization | merization | merization | merization |
| | | (T0: 0.1%) | (TO: 0.2%) | (TO: 0.1%) | (TO: 0.2%) |

¹Bold letters indicate CDR regions.
²clone 18C06 brings a different LC than clones 20H09, 23H09 and 24F2_N53A which share a common LC.

### Example 7: Crystal Dissolution Assay Results

All clones were evaluated for their capacity to dissolve GAL10 crystals in vitro. Briefly, a crystal dissolution assay (CDA) was developed and standardized for assessing the biological activity of the clones prior to and after application of several stress conditions including nebulization. The objective of this study was to evaluate if the nebulization prior/after storage could have an impact on the (bio) activity of the clones leading to a loss in their capacity to dissolve GAL10 crystals.

For all clones, the analysis was performed in two independent experiments with the presence of the appropriate assay controls. In brief, samples were nebulized with an Aerogen Solo active vibrating mesh nebulizer that converted a drug solution into an inhalable aerosol. Three different devices for each time-point were used per clone and results are reported by the device (nebulizer) serial number. In case of device fouling, indicated by longer nebulization times than usual for a given size of aliquot, spare devices of the same type were available to complete the nebulization with triplicate devices. For the nebulized candidates with triplicate devices, the aerosolized protein from one common device was analyzed as a 8-concentration points curve (Solo #0125) and the remaining two at a preselected, fixed concentration, always in two independent assays. Samples after 4 weeks storage at +5° C., +25° C. and +37° C. were analyzed as 8-concentration points curve for all clones. Results illustrated herein are taken from assays for which the size distribution of the GAL10 crystals were more than 10 μm (10⁻¹⁵ μm).

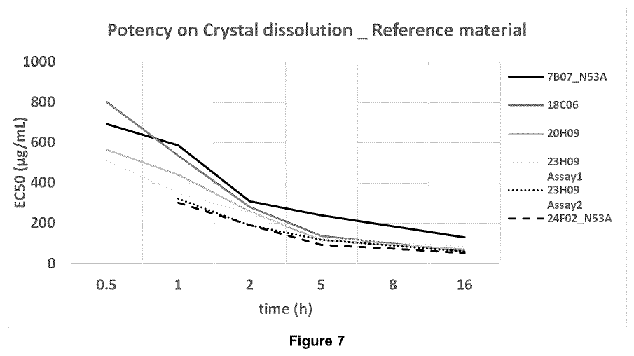

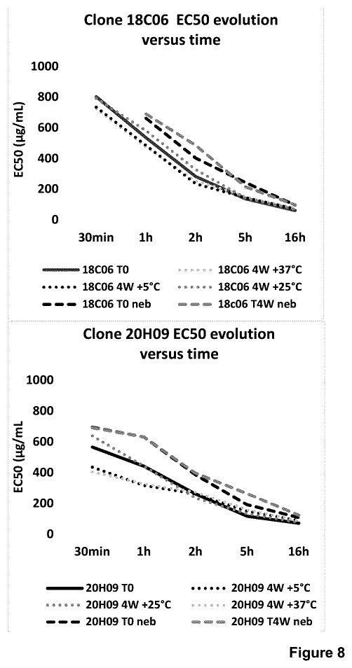

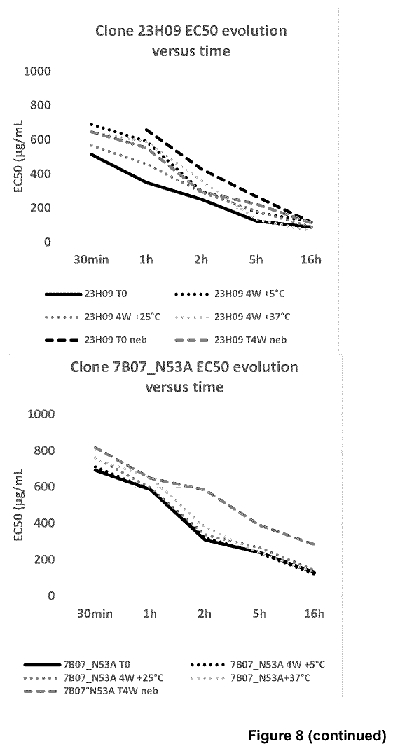

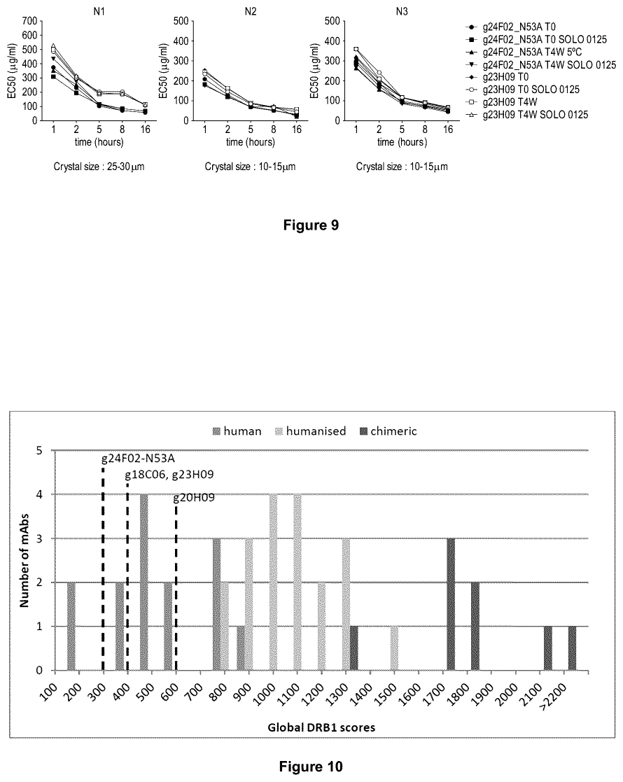

FIG. 7 illustrates the potency of unstressed samples containing one of the four clones to dissolve GAL10 crystals. All clones were then tested for potency after they had undergone the stress conditions described above to evaluate if they could preserve this potency after storage and/or after nebulization, prior- and post-storage (FIG. 8). Clones 18C06, 20H09 and 23H09 were tested in parallel in a first set of assays (FIG. 8) and clone 24F02_N53A was tested side-by-side with clone 23H09 in a second set of assays (FIG. 9). Clone 7B07_N53A was tested as a comparator in the first set of assays.

All clones were capable of fully dissolving the GAL10 crystals throughout the analytical run irrespective of the stressor applied to the sample (FIGS. 7-9). Consequently, further analysis of the clones focused mainly on the earliest time points up to 5h where a difference in potency between the clones was observable.

According to the assays, clones 23H09 and 24F02_N53A were the most potent molecules amongst the four clones (FIG. 9). For all independent runs, and irrespective of the size distribution of the GAL10 crystals tested, clone 24F02_N53A was more potent in terms of its ability to dissolve in vitro GAL10 crystals. Temperature or nebulization stress did not affect its capacity for dissolution when compared to the unstressed material (FIG. 9). Based on the above assays, the candidates were ranked from the most to least potent:
g24F02_N53˜g23H09>g20C06>g18C06

### Example 8: Immunogenicity of Candidate Clones

Endotoxin-free material for all clones was assessed for the immunogenicity risk using Lonza Epibase®, which includes an in silico assessment and a cell based in vitro assessment. Briefly, the in silico assessment utilized an algorithm to screen the amino acid sequences of the clones for potential immunogenic epitopes, including the allotypes that can be affected and the major histocompatibility complex by measuring the HLA-DRB1 score. The assay screened these allotypes next to their global population frequencies (FIG. 10). Based on the immunogenicity score, the clones were ranked from the least to the most immunogenic:
924F02_N53< [g23H09, g18C06]<g20H09

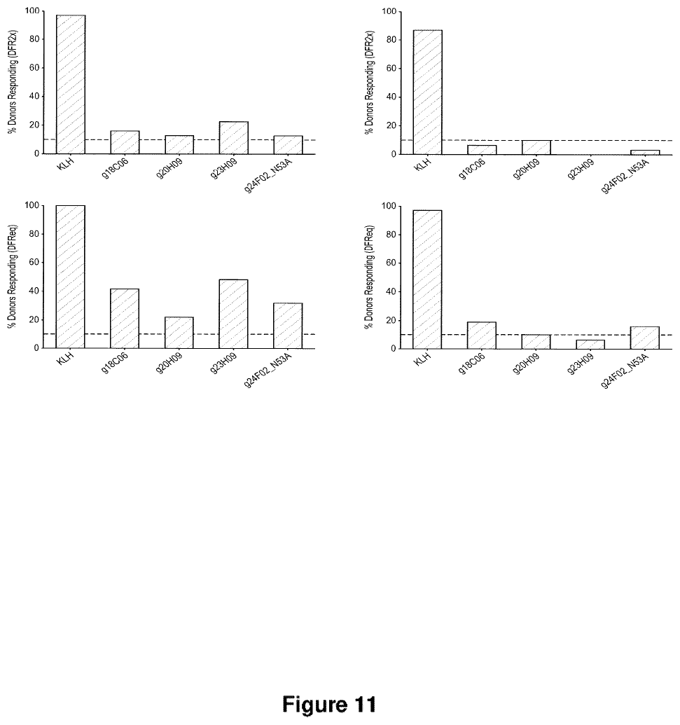

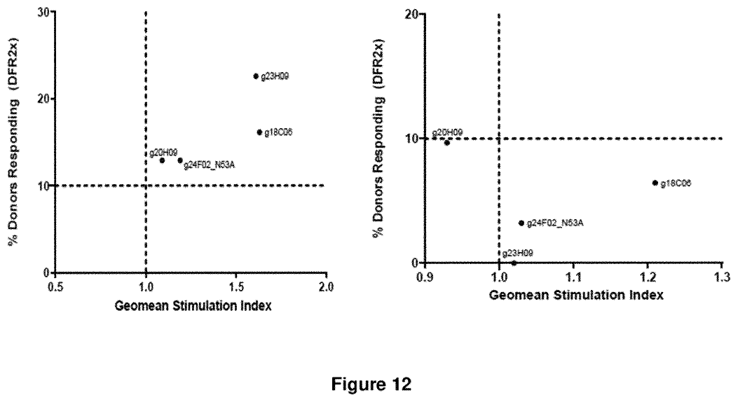

These results were confirmed with the Lonza Epibase® in vitro assay. Briefly, the candidates were evaluated for T-cell responses induced in PBMCs from 31 healthy donors. Screening was evaluated upon detection and enumeration of stimulated IFNγ and IL-5 cells to determine the number of donors eliciting a T-cell response as an unwanted immune response risk (FIG. 11), and the magnitude over the test population (FIG. 12). The positive control used for this study was clone KLH (FIG. 11).

All tested clones exhibited a low capacity to induce immune responses. Amongst the four clones, in terms of IFNγ response, clones 23H09 and 18C06 demonstrated the highest frequency while in terms of IL-5 responses, clones 18C06 and 20H09 demonstrated the highest frequency. In all instances and with all statistical approaches, clone g24F02_N53A was considered to be the least likely to induce an unwanted T-cell response and thus, this clone is considered to have the lowest risk of immunogenicity.

### Conclusion from Examples

Of the clones studied in detail, the germlined Fab clone 24F02_N53A was considered to be the most promising clone because:
It remained stable even after samples were subjected to elevated storage temperatures, multiple cycles of freeze-thawing and subjected to low pH conditions;
It exhibited strong binding to Galectin-10;
It was able to quickly dissolve recombinant Gal10 crystals in vitro;
It cross-reacted with cynomolgus monkey Gal10;
It is predicated to have a low immunogenicity risk in humans; and
The stability and binding properties of this clone were not impacted by nebulization.

TABLE 42
VH and VL sequences of Fab antibodies binding to galectin-10

| | | SEQ ID | | SEQ ID |
|---|---|---|---|---|
| Clone | V_{H} | NO. | V_{L} | NO. |
| g24F02_ | EVQLLESGGGLVQPGGSLRLSCAAS | 4 | SYELTQPLSVSVALGQTARITCDGNNIGSK | 10 |
| N53A | GFTFSSYAMSWVRQAPGKGLEWVSA | | SAQWYQQKPGQAPVLVIYADEYRPEGIPE | |
| | INAGGGSTSYADSVKGRFTISRDNSK | | RFSGSNSGNTATLTISRAQAGDEADYYCQ | |
| | NTLYLQMNSLRAEDTAVYYCAKGLRV | | VWDGSAAVFGGGTQLTVL | |
| | GVLGFDYWGQGTLVTVSS | | | |
| g24F02 | EVQLLESGGGLVQPGGSLRLSCAAS | 6 | SYELTQPLSVSVALGQTARITCDGNNIGSK | 10 |
| | GFTFSSYAMSWVRQAPGKGLEWVSA | | SAQWYQQKPGQAPVLVIYADEYRPEGIPE | |
| | INNGGGSTSYADSVKGRFTISRDNSK | | RFSGSNSGNTATLTISRAQAGDEADYYCQ | |
| | NTLYLQMNSLRAEDTAVYYCAKGLRV | | VWDGSAAVFGGGTQLTVL | |
| | GVLGFDYWGQGTLVTVSS | | | |
| g23H09 | EVQLLESGGGLVQPGGSLRLSCAAS | 14 | SYELTQPLSVSVALGQTARITCDGNNIGSK | 10 |
| | GFTFRSYDMSWVRQAPGKGLEWVS | | SAQWYQQKPGQAPVLVIYADEYRPEGIPE | |
| | AIAQFQHWTYYADSVKGRFTISRDNS | | RFSGSNSGNTATLTISRAQAGDEADYYCQ | |
| | KNTLYLQMNSLRAEDTAVYYCATTHG | | VWDGSAAVFGGGTQLTVL | |
| | IGTLGFGSWGQGTLVTVSS | | | |
| g18C06 | EVQLLESGGGLVQPGGSLRLSCAAS | 18 | QSVLTQPPSVSGAPGQRVTISCTGSSSNI | 22 |
| | GFTFSSFAMSWVRQAPGKGLEWVSG | | GSGSYLSWYQQLPGTAPKLLIYNANNRAS | |
| | INSGDGSSVKGRFTISRDNSKNTLYLQ | | GVPDRFSGSKSGTSASLAITGLQAEDEAD | |
| | MNSLRAEDTAVYYCAKGGATVWGRF | | YYCGCYDSKLSTYVFGGGTKLTVL | |
| | HDYWGQGTLVTVSS | | | |
| g20H09 | EVQLLESGGGLVQPGGSLRLSCAAS | 24 | SYELTQPLSVSVALGQTARITCDGNNIGSK | 10 |
| | GFTFRSYDMSWVRQAPGKGLEWVS | | SAQWYQQKPGQAPVLVIYADEYRPEGIPE | |
| | AIKWHRITYYADSVKGRFTISRDNSKN | | RFSGSNSGNTATLTISRAQAGDEADYYCQ | |
| | TLYLQMNSLRAEDTAVYYCATTHGIG | | VWDGSAAVFGGGTQLTVL | |
| | TLGFGSWGQGTLVTVSS | | | |

TABLE 43
Heavy chain CDR sequences of Fab antibodies binding to galectin-10

| | | SEQ | | SEQ | | SEQ |
|---|---|---|---|---|---|---|
| clone | CDR1 | ID NO. | CDR2 | ID NO. | CDR3 | ID NO. |
| g24F02_ | SYAMS | 1 | AINAGGGSTSYADSVKG | 3 | GLRVGVLGFDY | 2 |
| N53A | | | | | | |
| g24F02 | SYAMS | 1 | AINNGGGSTSYADSVKG | 5 | GLRVGVLGFDY | 2 |
| g23H09 | SYDMS | 11 | AIAQFQHWTYYADSVKG | 13 | THGIGTLGFGS | 12 |
| g18C06 | SFAMS | 15 | GINSGDGSSVKG | 17 | GGATVWGRFHDY | 16 |
| g20H09 | SYDMS | 11 | AIKWHRITYYADSVKG | 23 | THGIGTLGFGS | 12 |

TABLE 44
Light chain CDR sequences of Fab antibodies binding to galectin-10

| | | SEQ | | SEQ | | SEQ |
|---|---|---|---|---|---|---|
| clone | CDR1 | ID NO. | CDR2 | ID NO. | CDR3 | ID NO. |
| g24F02_ | DGNNIGSKSAQ | 7 | ADEYRPE | 9 | QVWDGSAAV | 8 |
| N53A | | | | | | |
| g24F02 | DGNNIGSKSAQ | 7 | ADEYRPE | 9 | QVWDGSAAV | 8 |
| g23H09 | DGNNIGSKSAQ | 7 | ADEYRPE | 9 | QVWDGSAAV | 8 |
| g18C06 | TGSSSNIGSGSYLS | 19 | NANNRAS | 21 | GCYDSKLSTYV | 20 |
| g20H09 | DGNNIGSKSAQ | 7 | ADEYRPE | 9 | QVWDGSAAV | 8 |

### REFERENCES

1. Selroos O, Kupczyk M, Kuna P, et al. National and regional asthma programmes in Europe. Eur Respir Rev. 2015; 24 (137): 474-483.
2. Braido F. Failure in asthma control: reasons and consequences. Scientifica (Cairo). 2013; 2013:549252.
3. Zhang L, He L, Gong J, Liu C. Risk Factors Associated with Irreversible Airway Obstruction in Asthma: A Systematic Review and Meta-Analysis. Biomed Res Int. 2016; 2016:9868704.
4. Lambrecht B N, Hammad H. The immunology of asthma. Nat Immunol. 2015; 16 (1): 45-56.
5. Persson E K, Verstraete K, Heyndrickx I, et al. Protein crystallization promotes type 2 immunity and is reversible by antibody treatment. Science. 2019; 364 (6442).
6. Butterfield J H, Ackerman S J, Scott R E, Pierre R V, Gleich G J. Evidence for secretion of human eosinophil granule major basic protein and Charcot-Leyden crystal protein during eosinophil maturation. Exp Hematol. 1984; 12 (3): 163-170.
7. Acharya K R, Ackerman S J. Eosinophil granule proteins: form and function. J Biol Chem. 2014; 289 (25): 17406-17415.
8. Leonidas D D, Elbert B L, Zhou Z, Leffler H, Ackerman S J, Acharya K R. Crystal structure of human Charcot-Leyden crystal protein, an eosinophil lysophospholipase, identifies it as a new member of the carbohydrate-binding family of galectins. Structure. 1995; 3 (12): 1379-1393.
9. Liu F T. Regulatory roles of galectins in the immune response. Int Arch Allergy Immunol. 2005; 136 (4): 385-400.
10. Weller P F, Bach D S, Austen K F. Biochemical characterization of human eosinophil Charcot-Leyden crystal protein (lysophospholipase). J Biol Chem. 1984; 259 (24): 15100-15105.
11. Ueki S, Tokunaga T, Melo RCN, et al. Charcot-Leyden crystal formation is closely associated with eosinophil extracellular trap cell death. Blood. 2018; 132 (20): 2183-2187.
12 el-Hashimi W. Charcot-Leyden crystals. Formation from primate and lack of formation from nonprimate eosinophils. Am J Pathol. 1971; 65 (2): 311-324.
13. Rabinovich G A, Rubinstein N, Fainboim L. Unlocking the secrets of galectins: a challenge at the frontier of glyco-immunology. J Leukoc Biol. 2002; 71 (5): 741-752.
14. Su J. A Brief History of Charcot-Leyden Crystal Protein/Galectin-10 Research. Molecules. 2018; 23 (11).

1. An antibody or antigen binding fragment thereof that binds to galectin-10, wherein the antibody or antigen binding fragment comprises a heavy chain variable domain (VH) and a light chain variable domain (VL) wherein:
(i) the VH comprises ta HCDR3 comprising the amino acid sequence of SEQ ID NO: 2; a HCDR2 comprising the amino acid sequence of SEQ ID NO: 3; and a HCDR1 comprising the amino acid sequence of SEQ ID NO: 1; and
(ii) the VL comprises a LCDR3 comprising the amino acid sequence of SEQ ID NO: 8; a LCDR2 comprising the amino acid sequence of SEQ ID NO: 9; and a LCDR1 comprising the amino acid sequence of SEQ ID NO: 7.

2. The antibody or antigen binding fragment thereof of claim 1, wherein:
(i) the VH comprises the amino acid sequence of SEQ ID NO: 4 or an amino acid sequence at least 90% identical thereto; and
(ii) the VL comprises the amino acid sequence of SEQ ID NO: 10 or an amino acid sequence at least 90% identical thereto.

3. The antibody or antigen binding fragment thereof of claim 1, wherein;
(i) the VH comprises the amino acid sequence of SEQ ID NO: 4; and
(ii) the VL comprises the amino acid sequence of SEQ ID NO: 10.

4. The antibody or antigen binding fragment thereof of claim 1, wherein the antigen binding fragment thereof is selected from the group consisting of: a single chain antibody (scFv); a F(ab′)2 fragment; a Fab fragment; an Fd fragment; an Fv fragment; a one-armed (monovalent) antibody; diabodies, triabodies, tetrabodies, or any antigen binding molecule formed by combination, assembly or conjugation of such antigen binding fragments.

5. The antibody or antigen binding fragment thereof of claim 4, wherein the antigen binding fragment thereof is a Fab fragment.

6. An isolated polynucleotide or polynucleotides which encode the antibody or antigen binding fragment thereof of claim 1, or a VH or VL domain thereof.

7. An expression vector comprising the polynucleotide of claim 6 operably linked to regulatory sequences which permit expression of the antibody, antigen binding fragment thereof, VH, or VL in a host cell or cell-free expression system.

8. A host cell or cell-free expression system containing the expression vector of claim 7.

9. A method of producing the antibody or antigen binding fragment thereof of claim 1, the method comprising culturing a host cell or cell free expression system under conditions which permit expression of the antibody or antigen binding fragment and recovering the expressed antibody or antigen binding fragment.

10. A pharmaceutical composition comprising the antibody or antigen binding fragment thereof according to claim 1, and at least one pharmaceutically acceptable carrier or excipient.

11-15. (canceled)

16. A method of treating a subject in need thereof, wherein the method comprises administering to the subject a therapeutically effective amount of the antibody or antigen binding fragment thereof according to claim 1.

17. The method of claim 16, wherein the antibody or antigen binding fragment thereof is administered to treat a disease or condition associated with the presence or formation of galectin-10 crystals.

18. The method of claim 16, wherein the antibody or antigen binding fragment thereof is administered to treat a disease or condition selected from the group consisting of: asthma; chronic rhinosinusitis; celiac disease; helminth infection; gastrointestinal eosinophilic inflammation; cystic fibrosis (CF); allergic bronchopulmonary aspergillosis (ABPA); Churg-Straus vasculitis; chronic eosinophilic pneumonia; and acute myeloid leukemia (AML).

19. The method of claim 18, wherein the disease or condition is asthma.

20. The method of claim 18, wherein the disease or condition is CF.

21-23. (canceled)
